(19) United States
(12) Patent Application Publication
(10) Pub. No.: US 2008/0132562 A1
(43) Pub. Date: Jun. 5, 2008

(54) TETRONIC AND TETRAMIC ACIDS

(76) Inventors: Thierry Godel, Basel (CH); Hans Hilpert, Muenchenstein (CH); Roland Humm, Freiburg (DE); Mark Rogers-Evans, Binningen (CH); Didier Rombach, Mulhouse (FR); Christopher Martin Stahl, Freiburg (DE); Peter Weiss, Basel (CH); Wolfgang Wostl, Grenzach-Wyhlen (DE)

Correspondence Address: HOFFMANN-LA ROCHE INC.;PATENT LAW DEPARTMENT, 340 KINGSLAND STREET, NUTLEY, NJ, 07110, US

(21) Appl. No.: 12/023,063

(22) Filed: Jan. 31, 2008

Related U.S. Application Data
(62) Division of application No. 10/994,823, filed on Nov. 22, 2004.

(30) Foreign Application Priority Data
Nov. 28, 2003 (EP) 03104437.3

(51) Int. Cl. A61K 31/40; A61P 25/28; A61K 31/34

(52) U.S. Cl. 514/423; 514/474

(57) ABSTRACT

This invention relates to new tetronic and tetramic acid derivatives with beta-secretase inhibitory activity of formula I:

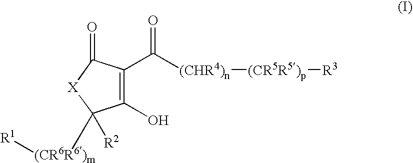

wherein R1, R2, R3, R4, R5, R5′, R6 and R6′ are as defined hereinabove, to processes for their preparation, compositions containing said tetronic and tetramic acid derivatives and their use in the treatment and prevention of diseases modulated by an inhibitor of β-secretase, such as Alzheimer's disease.

### PRIORITY TO RELATED APPLICATIONS

This application is a division of U.S. application Ser. No. 10/994,823, filed Nov. 22, 2004, now pending; which claims the benefit of European Application No. 03104437.3, filed Nov. 28, 2003. The entire contents of the above-identified applications are hereby incorporated by reference.

### BACKGROUND OF THE INVENTION

Alzheimer's disease (AD) is the most common cause of dementia in later life. Pathologically AD is characterized by the deposition in the brain of amyloid in extracellular plaques and intracellular neurofibrillary tangles. The amyloid plaques are mainly composed of amyloid peptides (Abeta peptides) which originate from the β-Amyloid Precursor Protein (APP) by a series of proteolytic cleavage steps. Several forms of APP have been identified of which the most abundant are proteins of 695, 751 and 770 amino acids length. They all arise from a single gene through differential splicing. The Abeta peptides are derived from the same domain of the APP but differ at their N- and C-termini, the main species are of 40 and 42 amino-acid length.

Abeta peptides are produced from APP through the sequential action of 2 proteolytic enzymes termed β- and γ-secretase. β-Secretase cleaves first in the extracellular domain of APP just outside of the trans-membrane domain (TM) to produce a C-terminal fragment of APP containing the TM- and cytoplasmatic domain (CTFβ). CTFβ is the substrate for γ-secretase which cleaves at several adjacent positions within the TM to produce the Aβ peptides and the cytoplasmic fragment. The β-Secretase is a typical aspartyl protease.

It is hypothesized that inhibiting the production of A-beta will prevent and reduce neurological degeneration, by controlling the formation of amyloid plaques, reducing neurotoxicity and, generally, mediating the pathology associated with A-beta production. Compounds that inhibit beta- or gamma-secretase activity, either directly or indirectly, could control the production of A-beta.

### SUMMARY OF THE INVENTION

This invention relates to new tetronic and tetramic acid derivatives with beta-secretase inhibitory activity, processes for their preparation, compositions containing said tetronic and tetramic acid derivatives and their use in the treatment and prevention of diseases.

The present invention provides a compound of the formula I

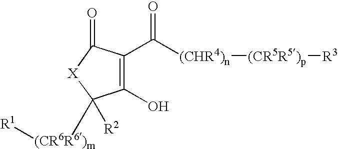

wherein

### X is O or NH;

R¹ is lower alkyl, cycloalkyl, heterocycloalkyl or aryl, wherein the aryl ring is unsubstituted or substituted by benzyloxy;
R² is H, lower alkyl or aryl;
R³ is lower alkyl,

—SCH₃,

acetyl,

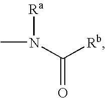

wherein R^{a} is H or lower alkyl, R^{b} is lower alkyl, heteroaryl, —OC(CH₃)₃ or aryl, wherein the aryl ring is unsubstituted or substituted by lower alkyl,

cycloalkyl, wherein the cycloalkyl ring is unsubstituted or substituted by lower alkyl or aryl,

heterocycloalkyl, wherein the heterocycloalkyl ring is unsubstituted or substituted by —COOC(CH₃)₃, or

(CH═CR′)ₒ-aryl, wherein the aryl ring is unsubstituted or substituted by lower alkyl, alkoxy, hydroxyl, benzyloxy, halogen, acetyl, —(CH₂)₂NHSO₂Ph, —NHCO(CH₂)₂NHCOOC(CH₃)₃, —(CH₂)₂NHCOC₆H₃OCH₃Cl, or for the non aromatic part of fused ring system also by oxo,

o is 0 or 1;

### R′ is H,

lower alkyl,

aryloxy, wherein the aryl ring is unsubstituted or substituted by lower alkyl or alkoxy, or

(CH═CH)_{q}-heteroaryl, wherein the heteroaryl ring is unsubstituted or substituted by lower alkyl, acetyl, alkoxy, halogen, —COOC(CH₃)₃ or by halogen substituted benzyl; or for the non aromatic part of fused ring system also by oxo;

q is 0 or 1;
R⁴ is H, lower alkyl, —(CH₂)₂SCH₃, —NHCOCH₃, —NHSO₂p-Cl-Ph, amino, —NHCOOC(CH₃)₃, hydroxyl, aryl, benzyl or halogen substituted benzyl;
R⁵ and R^{5′} are each independently selected from H, lower alkyl or aryl;
R⁶ and R^{6′} are each independently selected from H, lower alkyl or —SCH₃;
m is 1, 2 or 3;
n is 0 or 1; and
p is 0, 1, 2 or 3;
or a pharmaceutically acceptable salt thereof,
with the exception that the compound is not 3-acetyl-4-hydroxy-5-isobutyl-1,5-dihydro-pyrrol-2-one or 3-acetyl-5-benzyl-4-hydroxy-1,5-dihydro-5H-furan-2-one.

Compounds of 3-acetyl-4-hydroxy-5-isobutyl-1,5-dihydro-pyrrol-2-one and 3-acetyl-5-benzyl-4-hydroxy-1,5-dihydro-5H-furan-2-one are disclosed in EP 0841063 A1. The compounds are claimed in the European Patent Application to be effective in preventing and treating cytopenia caused by cancer chemotherapy, radiation therapy, and the like.

The present invention also provides for all forms of enantiomers, racemates or diastereomeric mixtures of compounds of formula I.

The present invention further provides pharmaceutical compositions that comprise a therapeutically effective amount of a compound of the invention and a pharmaceutically acceptable carrier, as well as methods of manufacturing such compositions.

The compounds of the present invention block the activity of β-secretase, reducing or preventing the formation of A-beta peptides. Thus, the present invention also provides methods for the treatment of diseases in which β-secretase plays a role. In particular, the present invention provides a method for the treatment of CNS diseases, such as Alzheimer's disease.

### DETAILED DESCRIPTION OF THE INVENTION

Unless otherwise stated, the following terms used in this Application have the definitions given below. The following definitions of general terms used herein apply irrespective of whether the terms in question appear alone or in combination. It must be noted that, as used in the description and the claims, the singular forms “a”, “an” and “the” include plural referents unless the context clearly dictates otherwise.

“Alkyl” means the monovalent linear or branched saturated hydrocarbon moiety, consisting solely of carbon and hydrogen atoms, having from one to twelve carbon atoms.

“Lower alkyl” refers to an alkyl group of one to six carbon atoms. Examples of alkyl groups include, but are not limited to, methyl, ethyl, propyl, isopropyl, n-butyl, isobutyl, sec-butyl, tert-butyl, pentyl, n-hexyl, octyl, dodecyl, and the like or those which are specifically exemplified herein.

“Alkoxy” means a moiety of the formula —OR^{z}, wherein R^{z} is an alkyl moiety as defined herein. Examples of alkoxy moieties include, but are not limited to, methoxy, ethoxy, isopropoxy, and the like or those which are specifically exemplified herein.

“Aryl” means a mono-, bi- or tricyclic aromatic radical consisting of one or more fused rings, in which at least one ring is aromatic in nature. The aryl group can optionally be substituted with one, two, three or four substituents, wherein each substituent independently is selected from hydroxy, cyano, alkyl, alkoxy, thiol, thioalkyl, halo, haloalkyl, nitro, amino, monoalkylamino, phenyloxy, benzyloxy, acetyl, (CH₂)₂NHSO₂Ph, —NHCO(CH₂)₂NHCOOC(CH₃)₃, —(CH₂)₂NHCOC₆H₃OCH₃Cl or for the non aromatic part of the fused ring system also by oxo, unless otherwise specifically indicated. Examples of aryl moieties include, but are not limited to, optionally substituted phenyl, optionally substituted naphthyl, optionally substituted 10,11-dihydro-5H-dibenzo[a,d]cyclohepten-5yl, optionally substituted 9H-fluoren-9-yl, optionally substituted indan-1-yl and the like or those which are specifically exemplified herein.

“Aryloxy” means a moiety of the formula —OR^{y}, wherein R^{y} is an aryl moiety as defined herein. Examples of aryloxy moieties include, but are not limited to, optionally substituted phenoxy and optionally substituted naphthoxy.

“Cycloalkyl” means a monovalent or divalent saturated carbocyclic moiety consisting of mono- or bicyclic rings. Cycloalkyl can optionally be substituted with one, two, three or four substituents, wherein each substituent is independently hydroxy, alkyl, alkoxy, halogen, amino, unless otherwise specifically indicated. Examples of cycloalkyl moieties include, but are not limited to, optionally substituted cyclopropyl, optionally substituted cyclobutyl, optionally substituted cyclopentyl, optionally substituted cyclopentenyl, optionally substituted cyclohexyl, optionally substituted cyclohexylene, optionally substituted cycloheptyl, and the like or those which are specifically exemplified herein.

“Halogen” refers to a substituent fluoro, chloro, bromo, or iodo.

“Heteroaryl” means a monocyclic, bicyclic or tricyclic radical of 5 to 12 ring atoms having at least one aromatic ring and furthermore containing one, two, or three ring heteroatoms selected from N, O, or S, the remaining ring atoms being C. Heteroaryl can optionally be substituted with one, two, three or four substituents, wherein each substituent is independently hydroxy, cyano, alkyl, alkoxy, thioalkyl, halo, haloalkyl, hydroxyalkyl, alkoxycarbonyl, amino, acetyl, —NHCOOC(CH₃)₃ or halogen substituted benzyl, or for the non aromatic part of cyclic ring also by oxo, unless otherwise specifically indicated. Examples of heteroaryl moieties include, but are not limited to, optionally substituted imidazolyl, optionally substituted oxazolyl, optionally substituted thiazolyl, optionally substituted pyrazinyl, optionally substituted pyrrolyl, optionally substituted pyrazinyl, optionally substituted pyridinyl, optionally substituted pyrimidinyl, optionally substituted indonyl, optionally substituted isoquinolinyl, optionally substituted carbazol-9-yl, optionally substituted furanyl, optionally substituted benzofuranyl, optionally substituted benzo[1,2,3]thiadiazolyl, optionally substituted benzo[b]thiophenyl, optionally substituted 9H-thioxanthenyl, optionally substituted thieno[2,3-c]pyridinyl and the like or those which are specifically exemplified herein.

“Heterocycloalkyl” means a monovalent saturated moiety, consisting of one, two or three rings, incorporating one, two, or three heteroatoms (chosen from nitrogen, oxygen or sulfur). Heterocycloalkyl can optionally be substituted with one, two, three or four substituents, wherein each substituent is independently hydroxy, alkyl, alkoxy, thioalkyl, halo, haloalkyl, hydroxyalkyl, alkoxycarbonyl, amino, alkylamino, dialkylamino, aminocarbonyl, or carbonylamino, unless otherwise specifically indicated. Examples of heterocyclic moieties include, but are not limited to, optionally substituted tetrahydro-furanyl, optionally substituted piperidinyl, optionally substituted pyrrolidinyl, optionally substituted morpholinyl, optionally substituted piperazinyl, and the like or those which are specifically exemplified herein.

“Pharmaceutically acceptable,” such as pharmaceutically acceptable carrier, excipient, etc., means pharmacologically acceptable and substantially non-toxic to the subject to which the particular compound is administered.

“Pharmaceutically acceptable salts” of a compound means salts that are pharmaceutically acceptable, as defined herein, and that possess the desired pharmacological activity of the parent compound. Such salts include: salts formed when an acidic proton present in the parent compound either is replaced by a metal ion, e.g., an alkali metal ion, an alkaline earth ion, or an aluminum ion; or coordinates with an organic or inorganic base. Acceptable organic bases include diethanolamine, ethanolamine, N-methylglucamine, triethanolamine, tromethamine, and the like. Acceptable inorganic bases include aluminum hydroxide, calcium hydroxide, potassium hydroxide, sodium carbonate and sodium hydroxide; or addition salts formed with inorganic acids such as hydrochloric acid, hydrobromic acid, sulfuric acid, nitric acid, phosphoric acid, and the like; or formed with organic acids such as acetic acid, benzenesulfonic acid, benzoic acid, camphorsulfonic acid, citric acid, ethanesulfonic acid, fumaric acid, glucoheptonic acid, gluconic acid, glutamic acid, glycolic acid, hydroxynaphthoic acid, 2-hydroxyethanesulfonic acid, lactic acid, maleic acid, malic acid, malonic acid, mandelic acid, methanesulfonic acid, muconic acid, 2-naphthalenesulfonic acid, propionic acid, salicylic acid, succinic acid, tartaric acid, p-toluenesulfonic acid, trimethylacetic acid, and the like.

“Therapeutically effective amount” means an amount that is effective to prevent, alleviate or ameliorate symptoms of disease or prolong the survival of the subject being treated.

“Thioalkyl” means a moiety of the formula —SR^{z}, wherein R^{z} is an alkyl moiety as defined herein.

“LDA” means lithiumdiisopropylamide.

“DCC” means dicyclohexyl carbodiimide.

“EDC” means N-(3-dimethylaminopropyl)-N′-ethyl carbodiimide hydrochloride.

“DMAP” means 4-dimethylamino pyridine.

“BOC” means t-butyloxycarbonyl.

The present invention provides a compound of the formula I

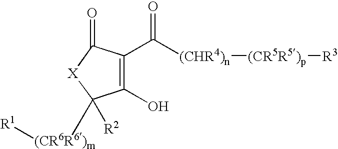

wherein

### X is O or NH;

R¹ is lower alkyl, cycloalkyl, heterocycloalkyl or aryl, wherein the aryl ring is unsubstituted or substituted by benzyloxy;
R² is H, lower alkyl or aryl;
R³ is lower alkyl,

—SCH₃,

acetyl,

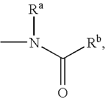

wherein R^{a} is H or lower alkyl, R^{b} is lower alkyl, heteroaryl, —OC(CH₃)₃ or aryl, wherein the aryl ring is unsubstituted or substituted by lower alkyl,

cycloalkyl, wherein the cycloalkyl ring is unsubstituted or substituted by lower alkyl or aryl,

heterocycloalkyl, wherein the heterocycloalkyl ring is unsubstituted or substituted by —COOC(CH₃)₃, or

(CH═CR′)ₒ-aryl, wherein the aryl ring is unsubstituted or substituted by lower alkyl, alkoxy, hydroxyl, benzyloxy, halogen, acetyl, —(CH₂)₂NHSO₂Ph, —NHCO(CH₂)₂NHCOOC(CH₃)₃, —(CH₂)₂NHCOC₆H₃OCH₃Cl, or for the non aromatic part of fused ring system also by oxo,

o is 0 or 1;

### R′ is H,

lower alkyl,

aryloxy, wherein the aryl ring is unsubstituted or substituted by lower alkyl or alkoxy, or

(CH═CH)_{q}-heteroaryl, wherein the heteroaryl ring is unsubstituted or substituted by lower alkyl, acetyl, alkoxy, halogen, —COOC(CH₃)₃ or by halogen substituted benzyl; or

for the non aromatic part of fused ring system also by oxo;
q is 0 or 1;
R⁴ is H, lower alkyl, —(CH₂)₂SCH₃, —NHCOCH₃, —NHSO₂p-Cl-Ph, amino, —NHCOOC(CH₃)₃, hydroxyl, aryl, benzyl or halogen substituted benzyl;
R⁵ and R^{5′} are each independently selected from H, lower alkyl or aryl;
R⁶ and R^{6′} are each independently selected from H, lower alkyl or —SCH₃;
m is 1, 2 or 3;
n is 0 or 1; and
p is 0, 1, 2 or 3;
or a pharmaceutically acceptable salt thereof,
with the exception that the compound is not 3-acetyl-4-hydroxy-5-isobutyl-1,5-dihydro-pyrrol-2-one or 3-acetyl-5-benzyl-4-hydroxy-1,5-dihydro-5H-furan-2-one.

In one embodiment the invention provides the compounds of the general formula Ia

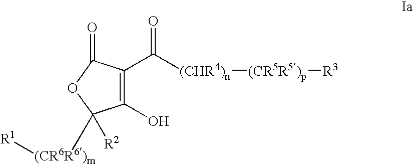

wherein
R¹ is lower alkyl, cycloalkyl, heterocycloalkyl or aryl, wherein the aryl ring is unsubstituted or substituted by benzyloxy;
R² is H, lower alkyl or aryl;
R³ is lower alkyl,

—SCH₃,

acetyl,

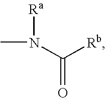

wherein R^{a} is H or lower alkyl, R^{b} is lower alkyl, heteroaryl, —OC(CH₃)₃ or aryl, wherein the aryl ring is unsubstituted or substituted by lower alkyl,

cycloalkyl, wherein the cycloalkyl ring is unsubstituted or substituted by lower alkyl or aryl,

heterocycloalkyl, wherein the heterocycloalkyl ring is unsubstituted or substituted by —COOC(CH₃)₃, or

(CH═CR′)ₒ-aryl, wherein the aryl ring is unsubstituted or substituted by lower alkyl, alkoxy, hydroxyl, benzyloxy, halogen, acetyl, —(CH₂)₂NHSO₂Ph, —NHCO(CH₂)₂NHCOOC(CH₃)₃, —(CH₂)₂NHCOC₆H₃OCH₃Cl, or for the non aromatic part of fused ring system also by oxo,

o is 0 or 1;

### R′ is H,

lower alkyl,

aryloxy, wherein the aryl ring is unsubstituted substituted by lower alkyl or alkoxy, or

(CH═CH)_{q}-heteroaryl, wherein the heteroaryl ring is unsubstituted or substituted by lower alkyl, acetyl, alkoxy, halogen, —COOC(CH₃)₃ or by halogen substituted benzyl; or for the non aromatic part of fused ring system also by oxo;

q is 0 or 1;
R⁴ is H, lower alkyl, —(CH₂)₂SCH₃, —NHCOCH₃, —NHSO₂p-Cl-Ph, amino, —NHCOOC(CH₃)₃, hydroxyl, aryl, benzyl or halogen substituted benzyl;
R⁵ and R^{5′} are each independently selected from H, lower alkyl or aryl;
R⁶ and R^{6′} are each independently selected from H, lower alkyl or —SCH₃;
m is 1, 2 or 3;
n is 0 or 1; and
p is 0, 1, 2 or 3;
or a pharmaceutically acceptable salt thereof,
with the exception that the compound is not 3-acetyl-5-benzyl-4-hydroxy-1,5-dihydro-5H-furan-2-one.

In another embodiment the present invention provides the compound of formula Ia, wherein

R¹ is lower alkyl, cycloalkyl, heterocycloalkyl, or aryl, wherein the aryl ring is unsubstituted or substituted by benzyloxy;
R² is H, lower alkyl or aryl;
R³ is lower alkyl, —SCH₃, acetyl,

cycloalkyl, wherein the cycloalkyl ring is unsubstituted or substituted by lower alkyl or aryl,

heterocycloalkyl,

(CH═CR′)ₒ-aryl, wherein the aryl ring is unsubstituted or substituted by lower alkyl, alkoxy, hydroxyl, benzyloxy, halogen, acetyl, —(CH₂)₂NHSO₂Ph, —NHCO(CH₂)₂NHCOOC(CH₃)₃ or —(CH₂)₂NHCOC₆H₆OCH₃Cl,
o is 0 or 1;

### R′ is H,

lower alkyl,

aryloxy, wherein the aryl ring is unsubstituted or substituted by lower alkyl or alkoxy, or

(CH═CH)_{q}-heteroaryl, wherein the heteroaryl ring is unsubstituted or substituted by lower alkyl, acetyl, alkoxy, halogen, or by halogen substituted benzyl;

q is 0 or 1;
R⁴ is H, lower alkyl, —(CH₂)₂SCH₃, —NHSO₂p-Cl-Ph, amino, —NHCOOC(CH₃)₃, hydroxyl, aryl, benzyl or halogen substituted benzyl;
R⁵ and R^{5′} are each independently selected from H, lower alkyl or aryl;
R⁶ and R^{6′} are each independently selected from H, lower alkyl or —SCH₃;
m is 1, 2 or 3;
n is 0 or 1; and
p is 0, 1, 2 or 3;
or a pharmaceutically acceptable salt thereof,
with the exception that the compound is not 3-acetyl-5-benzyl-4-hydroxy-1,5-dihydro-5H-furan-2-one.

In still another embodiment the present invention provides the compound of formula Ia, wherein

R¹ is methyl, cyclohexyl, phenyl, morpholin-4-yl or 4-benzyloxy-phenyl;
R² is H, methyl or phenyl;
R³ is methyl,

—SCH₃,

acetyl,

cycloalkyl, wherein the cycloalkyl ring is unsubstituted or substituted by methyl, tert-butyl or phenyl,

tetrahydro-furan-2-yl, pyrrolidine-2-yl, 1-tert-butyloxycarbonylpyrrolidine-2-yl, piperidine-2-yl, 1-tert-butyloxycarbonyl piperidine-2-yl,

(CH═CR′)ₒ-aryl, wherein the aryl ring is unsubstituted or substituted by methyl, tert-butyl, methoxy, hydroxyl, benzyloxy, chloro, fluoro, acetyl, —(CH₂)₂NHSO₂Ph, —NHCO(CH₂)₂NHCOOC(CH₃)₃, or —(CH₂)₂NHCO-3-chloro-2-methoxybenzene,

o is 0 or 1;

### R′ is H,

methyl,

aryloxy, wherein the aryl ring is unsubstituted or substituted by methyl or methoxy, or

(CH═CH)_{q}-heteroaryl, wherein the heteroaryl ring is unsubstituted or substituted by methyl, acetyl, methoxy, chloro, or by chloro or fluoro substituted benzyl;

q is 0 or 1;
R⁴ is H, methyl, ethyl, —(CH₂)₂SCH₃, —NHSO₂p-Cl-Phenyl, amino, —NHCOOC(CH₃)₃, hydroxyl, phenyl, benzyl or chloro substituted benzyl;
R⁵ and R^{5′} are each independently selected from H, methyl or phenyl;
R⁶ and R^{6′} are each independently selected from H, methyl or —SCH₃;
m is 1, 2 or 3;
n is 0 or 1; and
p is 0, 1, 2 or 3;
or a pharmaceutically acceptable salt thereof,
with the exception that the compound is not 3-acetyl-5-benzyl-4-hydroxy-1,5-dihydro-5H-furan-2-one.

In yet another embodiment the present invention provides the compound of formula Ia, wherein

R¹ is methyl, cyclohexyl, phenyl, morpholin-4-yl or 4-benzyloxy-phenyl;
R² is H, methyl or phenyl;
R³ is methyl, —SCH₃, acetyl, cyclopropanyl, 2,2,3,3-tetramethyl-cyclopropanyl, 2-phenyl-cyclopropanyl, cyclopent-2-enyl, cyclohexanyl, 4-tert-butyl-cyclohexanyl,

tetrahydro-furan-2-yl, pyrrolidine-2-yl, 1-tert-butyloxycarbonylpyrrolidine-2-yl piperidine-2-yl, 1-tert-butyloxycarbonylpiperidine-2-yl,

phenyl, 2-toluenyl, 3-toluenyl, 4-tert-butyl-phenyl, 4-fluoro-phenyl, 4-chloro-phenyl, 4-hydroxy-phenyl, 4-benzyloxy-phenyl, 2-methoxy-phenyl, 3-methoxy-phenyl, 4-methoxy-phenyl, —CH═C-phenyl, 2,4-dimethoxy-phenyl, 2,5-dimethoxy-phenyl, 3,4-dimethoxy-phenyl, 3,5-dimethoxy-phenyl, 4,5-dimethoxy-phenyl, 4-methoxy-2-methyl-phenyl, 4-methoxy-3-methyl-phenyl, -phenyl-4-(CH₂)₂NHSO₂Ph, -phenyl-4-NHCO(CH₂)₂NHCOOC(CH₃)₃, -phenyl-4-(CH₂)₂NHCO-3-chloro-2-methoxybenzene, naphthalen-2-yl, 6-methoxy-naphthalen-2-yl, 2-acetyl-naphthalen-1-yl, 10,11-dihydro-5H-dibenzo[a,d]cyclohepten-5-yl, 9H-fluoren-9-yl,

phenoxy, 3-dimethyl-phenoxy, 2,3-dimethyl-phenoxy, 2-methoxy-phenoxy, 3-methoxy-phenoxy, naphthalene-1-yloxy, or

—CH═CH-pyridin-3-yl, indol-1-yl, 1H-indol-3-yl, 1-methyl-1H-indol-3-yl, 4-fluoro-benzyl-1H-indol-3-yl, 1-(4-chloro-benzyl)-5-methoxy-2-methyl-1H-indol-3-yl, 1-(4-chloro-benzoyl)-5-methoxy-2-methyl-1H-indol-3-yl, 2-acetyl-1,2-dihydro-isoquinolin-1-yl, 1,2,3,4-tetrahydro-isoquinoline-2-yl, (3,4-dihydro-1H-isoquinoline-2-carboxylic acid tert-butyl ester)-3-yl, 2-methyl-benzofuran-3-yl, 5-chloro-benzofuran-3-yl, benzo[b]thiophen-3-yl, or 9H-thioxanthene-9-yl,

R⁴ is H, methyl, ethyl, —(CH₂)₂SCH₃, —NHSO₂p-Cl-Phenyl, amino, —NHCOOC(CH₃)₃, hydroxyl, phenyl, benzyl or chloro substituted benzyl;
R⁵ and R^{5′} are each independently selected from H, methyl or phenyl;
R⁶ and R^{6′} are each independently selected from H, methyl or —SCH₃;
m is 1, 2 or 3;
n is 0 or 1; and
p is 0, 1, 2 or 3;
or a pharmaceutically acceptable salt thereof,
with the exception that the compound is not 3-acetyl-5-benzyl-4-hydroxy-1,5-dihydro-5H-furan-2-one

Still in another embodiment the present invention provides the compound of general formula Ib

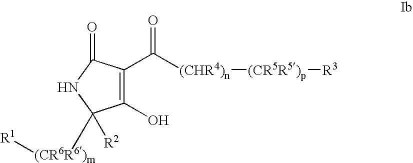

wherein
R¹ is lower alkyl, cycloalkyl, heterocycloalkyl or aryl, wherein the aryl ring is unsubstituted or substituted by benzyloxy;
R² is H, lower alkyl or aryl;
R³ is lower alkyl,

—SCH₃,

acetyl,

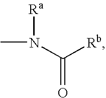

wherein R^{a} is H or lower alkyl, R^{b} is lower alkyl, heteroaryl, —OC(CH₃)₃ or aryl, wherein the aryl ring is unsubstituted or substituted by lower alkyl,

cycloalkyl, wherein the cycloalkyl ring is unsubstituted or substituted by lower alkyl or aryl,

heterocycloalkyl, wherein the heterocycloalkyl ring is unsubstituted or substituted by —COOC(CH₃)₃ or

(CH═CR′)ₒ-aryl, wherein the aryl ring is unsubstituted or substituted by lower alkyl, alkoxy, hydroxyl, benzyloxy, halogen, acetyl, —(CH₂)₂NHSO₂Ph, —NHCO(CH₂)₂NHCOOC(CH₃)₃, or —(CH₂)₂NHCOC₆H₆OCH₃Cl, or for the non aromatic part of fused ring system also by oxo,

o is 0 or 1;

### R′ is H,

lower alkyl,

aryloxy, wherein the aryl ring is unsubstituted or substituted by lower alkyl or alkoxy, or

(CH═CH)_{q}-heteroaryl, wherein the heteroaryl ring is unsubstituted or substituted by lower alkyl, acetyl, alkoxy, halogen, —COOC(CH₃)₃ or by halogen substituted benzyl; or for the non aromatic part of fused ring system also by oxo,

q is 0 or 1;
R⁴ is H, lower alkyl, —(CH₂)₂SCH₃, —NHCOCH₃, —NHSO₂p-Cl-Ph, amino, —NHCOOC(CH₃)₃, hydroxyl, aryl, benzyl or halogen substituted benzyl;
R⁵ and R^{5′} are each independently selected from H, lower alkyl or aryl;
R⁶ and R^{6′} are each independently selected from H, lower alkyl or —SCH₃;
m is 1, 2 or 3;
n is 0 or 1; and
p is 0, 1, 2 or 3;
or a pharmaceutically acceptable salt thereof,
with the exception that the compound is not 3-acetyl-4-hydroxy-5-isobutyl-1,5-dihydro-pyrrol-2-one.

Still yet in another embodiment the present invention provides the compound of formula Ib,

wherein
R¹ is aryl;

### R² is H;

### R³ is —SCH₃,

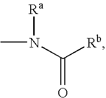

wherein R^{a} is H or lower alkyl, R^{b} is lower alkyl, heteroaryl, —OC(CH₃)₃ or aryl, wherein the aryl ring is unsubstituted or substituted by lower alkyl,

cycloalkyl, wherein the cycloalkyl ring is unsubstituted or substituted by lower alkyl,

heterocycloalkyl, wherein the heterocycloalkyl ring is unsubstituted or substituted by —COOC(CH₃)₃,

aryl, wherein the aryl ring is unsubstituted or substituted by lower alkyl, alkoxy, benzyloxy or for the non aromatic part of fused ring system also by oxo,

aryloxy, wherein the aryl ring is unsubstituted or substituted by alkoxy, or

heteroaryl, wherein the heteroaryl ring is unsubstituted or substituted by lower alkyl, —COOC(CH₃)₃ or by halogen substituted benzyl, or for the non aromatic part of fused ring system also by oxo;

R⁴ is H, lower alkyl, —NHCOCH₃, amino, —NHCOOC(CH₃)₃, aryl or benzyl;

### R⁵ and R^{5′} are H;

### R⁶ and R^{6′} are H;

m is 2;
n is 0 or 1; and
p is 0, 1, 2 or 3;
or a pharmaceutically acceptable salt thereof.

Yet in another embodiment the present invention provides the compound of formula Ib wherein

R¹ is phenyl;

### R² is H;

### R³ is —SCH₃,

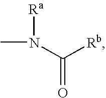

wherein R^{a} is H or methyl, R^{b} is methyl, 1H-pyrrol-3-yl, —OC(CH₃)₃ or aryl, wherein the aryl ring is unsubstituted or substituted by methyl,

cycloalkyl, wherein the cycloalkyl ring is unsubstituted or substituted by methyl,

heterocycloalkyl, wherein the heterocycloalkyl ring is unsubstituted or substituted by —COOC(CH₃)₃,

aryl, wherein the aryl ring is unsubstituted or substituted by methyl, tert-butyl, methoxy, benzyloxy or for the non aromatic part of fused ring system also by oxo,

aryloxy, wherein the aryl ring is substituted by methoxy, or

heteroaryl, wherein the heteroaryl ring is unsubstituted or substituted by methyl, —COOC(CH₃)₃ or by 4-fluoro-benzyl-1-yl, or for the non aromatic part of fused ring system also by oxo;

R⁴ is H, methyl, —NHCOCH₃, amino, —NHCOOC(CH₃)₃, phenyl or benzyl;

### R⁵ and R^{5′} are H;

### R⁶ and R^{6′} are H;

m is 2;
n is 0 or 1; and
p is 0, 1, 2 or 3;
or a pharmaceutically acceptable salt thereof.

Still yet in another embodiment the present invention provides the compound of formula Ib, wherein

R¹ is phenyl;

### R² is H;

R³ is —SCH₃, —NHCOCH₃, —NHCO-phenyl, —NHCO-(4-methyl-phenyl), —NHCO-(2,5-dihydro-1H-pyrrol-3-yl), NHCOOC(CH₃)₃,

cyclopropanyl, 1-methyl-cyclopropanyl, cyclohexanyl,

1-tert-butyloxycarbonylpyrrolidine-2-yl, 1-tert-butyloxycarbonylpiperidine-2-yl, tetrahydro-furan-2-yl,

phenyl, toluenyl, 4-tert-butyl-phenyl, 2-methoxy-phenyl, 3-methoxy-phenyl, 4-benzoxy-phenyl, 3,4-dimethoxy-phenyl, naphthalene-2-yl, 6-methoxy-naphthalen-2-yl, 3-oxo-indan-1-yl,

2-methyl-phenoxyl, or

1,2,5-trimethyl-1H-pyrrole-3-yl, 5-methyl-pyrazine-2-yl, 5-methyl-2,4-dioxo-1H-pyrimidine-1-yl, 3-methyl-furan-2-yl, indol-1-yl, 1H-indol-3-yl, (4-fluoro-benzyl)-1H-indol-3-yl, isoquinoline-3-yl, 3,4-dihydro-1H-isoquinoline-2-carboxylic acid ter-butyl ester, thieno[2,3-c]pyridine-7-yl, benzo[1,2,3]thiadiazole-5-yl, 2,3-dihydro-benzofuran-7-yl, 2-benzo[b]thiophen-3-yl, or carbazol-9-yl,

R⁴ is H, methyl, —NHCOCH₃, amino, —NHCOOC(CH₃)₃, phenyl or benzyl;

### R⁵ and R^{5′} are H;

### R⁶ and R^{6′} are H;

m is 2;
n is 0 or 1; and
p is 0, 1, 2 or 3;
or a pharmaceutically acceptable salt thereof.

Representative compounds of formula I in accordance with the present invention are shown in Table 1 below.

TABLE 1

| | |
|---|---|
| | (I) |
| 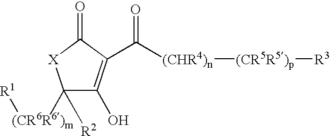 | |

| Ex | X | R¹ | —(CR⁶R^{6′})ₘ— | R² | —(CHR⁴)ₙ(CR⁵R^{5′})ₚ— | R³ |
|---|---|---|---|---|---|---|
| A1 | O | CH₃ | —CH(CH₃)CH₂— | H | —CH₂CH(CH₃)— | CH₃ |
| A2 | O | CH₃ | —CH(CH₃)CH₂— | H | —CH₂CH₂— | SCH₃ |
| A3 | O | CH₃ | —CH(CH₃)CH₂— | H | —CH₂CH₂CH(CH₃)— | CH₃ |
| A4 | O | CH₃ | —CH(CH₃)CH₂— | H | —CH(CH₃)CH₂— | —COCH₃ |
| A5 | O | CH₃ | —CH(CH₃)CH₂— | H | — | 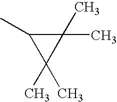 |
| A6 | O | CH₃ | —CH(CH₃)CH₂— | H | — | 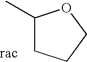 |
| A7 | O | CH₃ | —CH(CH₃)CH₂— | H | — | 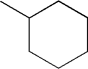 |
| A8 | O | CH₃ | —CH(CH₃)CH₂— | H | — | 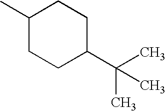 |
| A9 | O | CH₃ | —CH(CH₃)CH₂— | H | — | 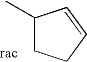 |
| A10 | O | CH₃ | —CH(CH₃)CH₂— | H | —CH₂— | 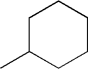 |
| A11 | O | CH₃ | —CH(CH₃)CH₂— | H | —CH₂CH₂CH₂— | 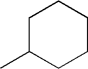 |
| A12 | O | CH₃ | —CH(CH₃)CH₂— | H | — | 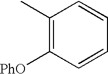 |
| A13 | O | CH₃ | —CH(CH₃)CH₂— | H | —CH₂— | 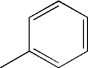 |
| A14 | O | CH₃ | —CH(CH₃)CH₂— | H | —CH₂— | 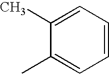 |
| A15 | O | CH₃ | —CH(CH₃)CH₂— | H | —CH₂— | 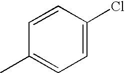 |
| A16 | O | CH₃ | —CH(CH₃)CH₂— | H | —CH₂— | 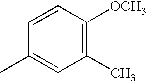 |
| A17 | O | CH₃ | —CH(CH₃)CH₂— | H | —CH₂— | 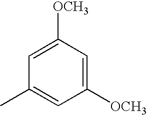 |
| A18 | O | CH₃ | —CH(CH₃)CH₂— | H | —CH₂— | 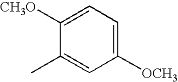 |
| A19 | O | CH₃ | —CH(CH₃)CH₂— | H | —CH₂— | 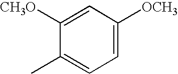 |
| A20 | O | CH₃ | —CH(CH₃)CH₂— | H | —CH(CH₃)— | 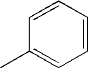 |
| A21 | O | CH₃ | —CH(CH₃)CH₂— | H | —CH(CH₂CH₃)— | 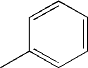 |
| A22 | O | CH₃ | —CH(CH₃)CH₂— | H | —CH(CH₃)— | 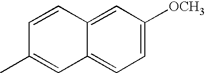 |
| A23 | O | CH₃ | —CH(CH₃)CH₂— | H | —CH₂CH₂— | 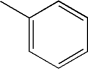 |
| A24 | O | CH₃ | —CH(CH₃)CH₂— | H | —CH₂CH₂— | 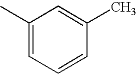 |
| A25 | O | CH₃ | —CH(CH₃)CH₂— | H | —CH₂CH₂— | 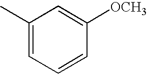 |
| A26 | O | CH₃ | —CH(CH₃)CH₂— | H | —CH₂CH₂— | 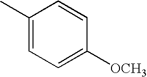 |
| A27 | O | CH₃ | —CH(CH₃)CH₂— | H | —CH₂CH₂— | 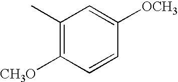 |
| A28 | O | CH₃ | —CH(CH₃)CH₂— | H | —CH(CH₃)CH₂— | 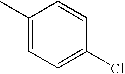 |
| A29 | O | CH₃ | —CH(CH₃)CH₂— | H | —CH(CH₃)CH₂— | 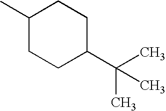 |
| A30 | O | CH₃ | —CH(CH₃)CH₂— | H | —CH₂CH(CH₃)— | 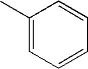 |
| A31 | O | CH₃ | —CH(CH₃)CH₂— | H | — | 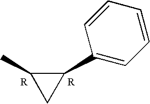 |
| A32 | O | CH₃ | —CH(CH₃)CH₂— | H | —CH₂— | 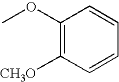 |
| A33 | O | CH₃ | —CH(CH₃)CH₂— | H | —CH₂— | 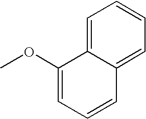 |
| A34 | O | CH₃ | —CH(CH₃)CH₂— | H | —CH(CH₃)— | 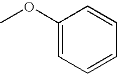 |
| A35 | O | CH₃ | —CH(CH₃)CH₂— | H | —CH₂CH₂CH₂— | 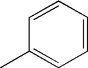 |
| A36 | O | CH₃ | —CH(CH₃)CH₂— | H | —CH₂CH₂CH₂— | 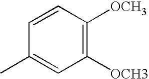 |
| A37 | O | CH₃ | —CH(CH₃)CH₂— | H | —CH(CH₃)CH₂— | 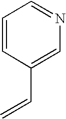 |
| A38 | O | CH₃ | —CH(CH₃)CH₂— | H | —CH(CH₃)CH₂— | 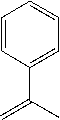 |
| A39 | O | CH₃ | —CH(CH₃)CH₂— | H | —CH₂— | 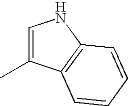 |
| A40 | O | CH₃ | —CH(CH₃)CH₂— | H | —CH₂CH₂— | 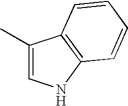 |
| A41 | O | CH₃ | —CH(CH₃)CH₂— | H | —CH₂— | 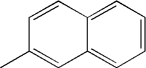 |
| A42 | O | CH₃ | —CH(CH₃)CH₂— | H | —CH₂— | 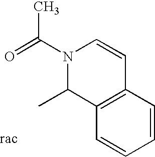 |
| A43 | O | CH₃ | —CH(CH₃)CH₂— | H | —CH(C₆H₅)— | 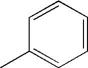 |
| A44 | O | CH₃ | —CH(CH₃)CH₂— | H | —CH(C₆H₅)CH₂— | 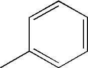 |
| A45 | O | CH₃ | —CH(CH₃)CH₂— | H | —CH₂— | 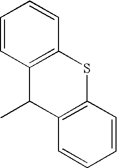 |
| A46 | O | CH₃ | —CH(CH₃)CH₂— | H | —CH₂— | 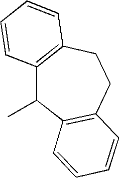 |
| B1 | O | CH₃ | —CH(SCH₃)CH₂— | H | —CH₂CH₂— | —SCH₃ |
| B2 | O | CH₃ | —CH(SCH₃)CH₂— | H | — | 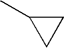 |
| B3 | O | CH₃ | —CH(SCH₃)CH₂— | H | — | 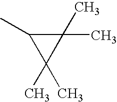 |
| B4 | O | CH₃ | —CH(SCH₃)CH₂— | H | — | 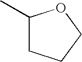 |
| B5 | O | CH₃ | —CH(SCH₃)CH₂— | H | — | 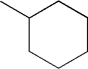 |
| B6 | O | CH₃ | —CH(SCH₃)CH₂— | H | — | 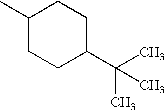 |
| IB7 | O | CH₃ | —CH(SCH₃)CH₂— | H | —CH₂— | 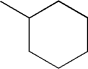 |
| B8 | O | CH₃ | —CH(SCH₃)CH₂— | H | —CH₂CH₂CH₂— | 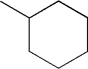 |
| B9 | O | CH₃ | —CH(SCH₃)CH₂— | H | —CH₂— | 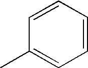 |
| B10 | O | CH₃ | —CH(SCH₃)CH₂— | H | —CH₂— | 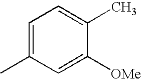 |
| B11 | O | CH₃ | —CH(SCH₃)CH₂— | H | —CH₂— | 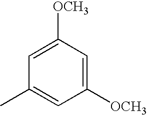 |
| B12 | O | CH₃ | —CH(SCH₃)CH₂— | H | —CH₂— | 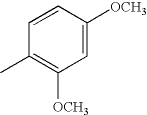 |
| B13 | O | CH₃ | —CH(SCH₃)CH₂— | H | —CH₂— | 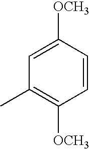 |
| B14 | O | CH₃ | —CH(SCH₃)CH₂— | H | —CH₂— | 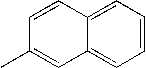 |
| B15 | O | CH₃ | —CH(SCH₃)CH₂— | H | —CH(CH₃)— | 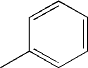 |
| B16 | O | CH₃ | —CH(SCH₃)CH₂— | H | —CH(CH₂CH₃)— | 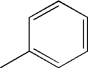 |
| B17 | O | CH₃ | —CH(SCH₃)CH₂— | H | —CH(CH₃)— | 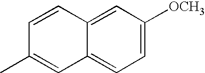 |
| IB18 | O | CH₃ | —CH(SCH₃)CH₂— | H | —CH₂CH₂— | 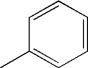 |
| B19 | O | CH₃ | —CH(SCH₃)CH₂— | H | —CH₂CH₂— | 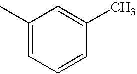 |
| B20 | O | CH₃ | —CH(SCH₃)CH₂— | H | —CH₂CH₂— | 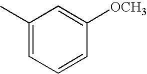 |
| B21 | O | CH₃ | —CH(SCH₃)CH₂— | H | —CH₂CH₂— | 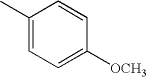 |
| B22 | O | CH₃ | —CH(SCH₃)CH₂— | H | —CH₂CH₂— | 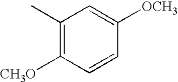 |
| B23 | O | CH₃ | —CH(SCH₃)CH₂— | H | —CH(CH₃)CH₂— | 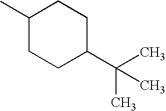 |
| B24 | O | CH₃ | —CH(SCH₃)CH₂— | H | —CH₂CH(CH₃)— | 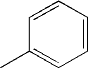 |
| B25 | O | CH₃ | —CH(SCH₃)CH₂— | H | — | 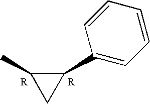 |
| B26 | O | CH₃ | —CH(SCH₃)CH₂— | H | —CH₂— | 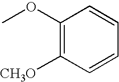 |
| B27 | O | CH₃ | —CH(SCH₃)CH₂— | H | —CH₂— | 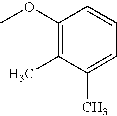 |
| B28 | O | CH₃ | —CH(SCH₃)CH₂— | H | —CH(CH₃)— | 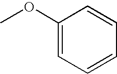 |
| B29 | O | CH₃ | —CH(SCH₃)CH₂— | H | —CH(CH₂CH₃)— | 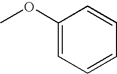 |
| B30 | O | CH₃ | —CH(SCH₃)CH₂— | H | —CH₂— | 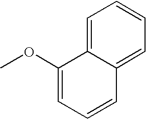 |
| B31 | O | CH₃ | —CH(SCH₃)CH₂— | H | —CH₂CH₂CH₂— | 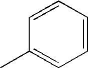 |
| B32 | O | CH₃ | —CH(SCH₃)CH₂— | H | —CH₂CH₂CH₂— | 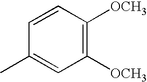 |
| B33 | O | CH₃ | —CH(SCH₃)CH₂— | H | —CH₂— | 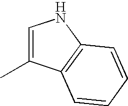 |
| B34 | O | CH₃ | —CH(SCH₃)CH₂— | H | —CH₂CH₂— | 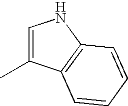 |
| B35 | O | CH₃ | —CH(SCH₃)CH₂— | H | —CH₂— | 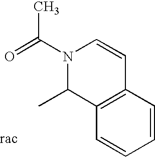 |
| B36 | O | CH₃ | —CH(SCH₃)CH₂— | H | —CH(C₆H₅)— | 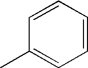 |
| B37 | O | CH₃ | —CH(SCH₃)CH₂— | H | —CH₂CH(C₆H₅)— | 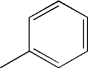 |
| B38 | O | CH₃ | —CH(SCH₃)CH₂— | H | —CH₂— | 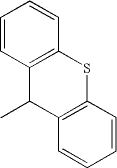 |
| B39 | O | CH₃ | —CH(SCH₃)CH₂— | H | —CH₂— | 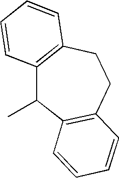 |
| C1 | O | cyclohexyl | —CH₂— | H | — | 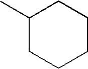 |
| C2 | O | cyclohexyl | —CH₂— | H | —CH₂— | 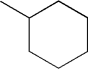 |
| C3 | O | cyclohexyl | —CH₂— | H | —CH₂CH₂— | 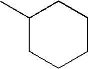 |
| C4 | O | cyclohexyl | —CH₂— | H | —CH₂CH₂CH₂— | 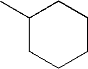 |
| C5 | O | cyclohexyl | —CH₂— | H | —CH(NHSO₂-4-Cl-Phenyl)CH₂CH₂— | 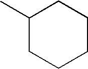 |
| C6 | O | cyclohexyl | —CH₂— | H | —CH₂CH₂CH₂CH₂— | 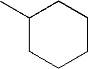 |
| C7 | O | cyclohexyl | —CH₂— | H | —CH(CH₃)CH₂— | 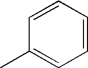 |
| C8 | O | cyclohexyl | —CH₂— | H | —CH(CH₃)CH₂— | 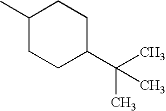 |
| C9 | O | cyclohexyl | —CH₂— | H | —CH(CH₃)CH₂— | 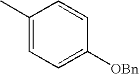 |
| C10 | O | cyclohexyl | —CH₂— | H | —CH(CH₃)CH₂— | 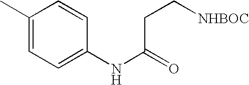 |
| C11 | O | cyclohexyl | —CH₂— | H | —CH(CH₃)CH₂— | 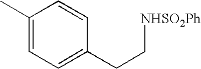 |
| C12 | O | cyclohexyl | —CH₂— | H | —CH(CH₃)CH₂— | 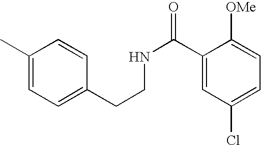 |
| C13 | O | cyclohexyl | —CH₂— | H | —CHNHBOCCH₂— | 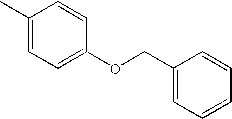 |
| C14 | O | cyclohexyl | —CH₂— | H | —CHNHCOCH₂— | 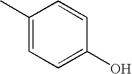 |
| C15 | O | cyclohexyl | —CH₂— | H | —CH(NH₂)CH₂— | 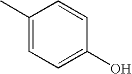 |
| C16 | O | cyclohexyl | —CH₂— | H | —CH₂— | 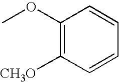 |
| C17 | O | cyclohexyl | —CH₂— | H | —CH₂— | 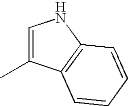 |
| C18 | O | cyclohexyl | —CH₂— | H | —CH₂— | 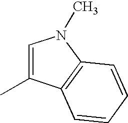 |
| C19 | O | cyclohexyl | —CH₂— | H | —CH₂— | 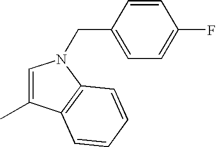 |
| C20 | O | cyclohexyl | —CH₂— | H | —CH₂— | 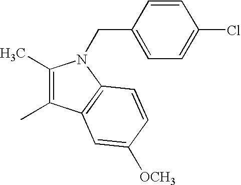 |
| C21 | O | cyclohexyl | —CH₂— | H | —CH₂— | 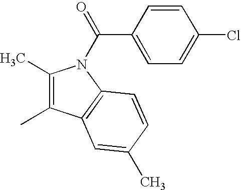 |
| C22 | O | cyclohexyl | —CH₂— | H | —CH₂— | 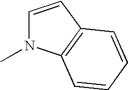 |
| C23 | O | cyclohexyl | —CH₂— | H | —CH₂CH₂— | 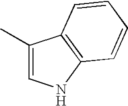 |
| C24 | O | cyclohexyl | —CH₂— | H | —CH₂— | 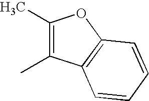 |
| C25 | O | cyclohexyl | —CH₂— | H | —CH₂— | 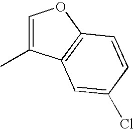 |
| C26 | O | cyclohexyl | —CH₂— | H | —CH₂— | 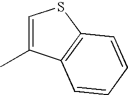 |
| C27 | O | cyclohexyl | —CH₂— | H | —CH₂CH(C₆H₅)— | —C₆H₅ |
| C28 | O | cyclohexyl | —CH₂— | H | —CH(C₆H₅)CH₂— | —C₆H₅ |
| C29 | O | cyclohexyl | —CH₂— | H | —CH(C₆H₅)CH₂— | 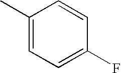 |
| C30 | O | cyclohexyl | —CH₂— | H | —CH(CH₂C₆H₅)CH₂— | —C₆H₅ |
| C31 | O | cyclohexyl | —CH₂— | H | —CH(CH₂C₆H₅-4-Cl)CH₂— | 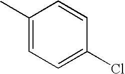 |
| C32 | O | cyclohexyl | —CH₂— | H | —CH₂— | 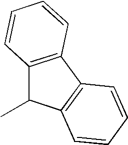 |
| C33 | O | cyclohexyl | —CH₂— | H | —CH₂— | 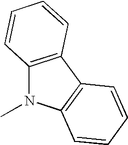 |
| D1 | O | C₆H₅— | —CH₂— | H | — | 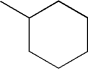 |
| D2 | O | C₆H₅— | —CH₂— | H | —CH(CH₃)CH₂— | 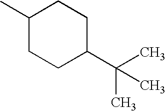 |
| D3 | O | C₆H₅— | —CH₂— | H | —CH₂— | 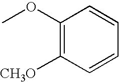 |
| D4 | O | C₆H₅— | —CH₂— | H | —CH₂CH₂CH₂— | 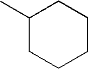 |
| D5 | O | C₆H₅— | —CH₂— | H | —CH₂— | 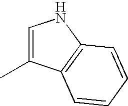 |
| D6 | O | C₆H₅— | —CH₂— | H | —CH(C₆H₅)CH₂— | 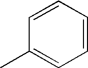 |
| D7 | O | C₆H₅— | —CH₂— | H | —CH₂— | 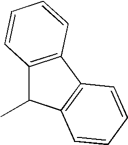 |
| E1 | O | C₆H₅— | —CH₂CH₂— | H | —CH₂CH₂— | —SCH₃ |
| E2 | O | C₆H₅— | —CH₂CH₂— | H | —CH(CH₃)— | —CH₃ |
| | | | | | CH₂CH(CH₃)— | |
| E3 | O | C₆H₅— | —CH₂CH₂— | H | —CH(CH₃)— | —CH₃ |
| | | | | | CH₂CH₂CH₂— | |
| E4 | O | C₆H₅— | —CH₂CH₂— | H | — | 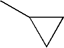 |
| E5 | O | C₆H₅— | —CH₂CH₂— | H | — | 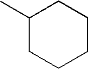 |
| E6 | O | C₆H₅— | —CH₂CH₂— | H | —CH₂— | 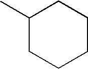 |
| E7 | O | C₆H₅— | —CH₂CH₂— | H | —CH₂CH₂CH₂— | 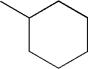 |
| E8 | O | C₆H₅— | —CH₂CH₂— | H | —CH₂— | 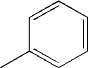 |
| E9 | O | C₆H₅— | —CH₂CH₂— | H | —CH₂— | 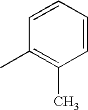 |
| E10 | O | C₆H₅— | —CH₂CH₂— | H | —CH(CH₃)— | 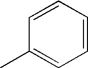 |
| E11 | O | C₆H₅— | —CH₂CH₂— | H | —CH(CH₂CH₃)— | 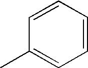 |
| E12 | O | C₆H₅— | —CH₂CH₂— | H | —CH₂— | 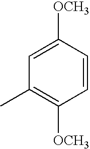 |
| E13 | O | C₆H₅— | —CH₂CH₂— | H | —CH₂— | 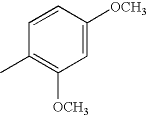 |
| E14 | O | C₆H₅— | —CH₂CH₂— | H | —CH₂— | 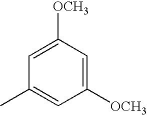 |
| E15 | O | C₆H₅— | —CH₂CH₂— | H | —CH₂CH₂— | 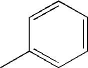 |
| E16 | O | C₆H₅— | —CH₂CH₂— | H | — | 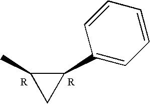 |
| E17 | O | C₆H₅— | —CH₂CH₂— | H | —CH₂CH(CH₃)— | 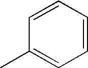 |
| E18 | O | C₆H₅— | —CH₂CH₂— | H | —CH(OH)CH₂— | 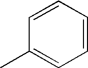 |
| E19 | O | C₆H₅— | —CH₂CH₂— | H | —CH₂CH₂— | 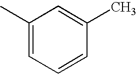 |
| E20 | O | C₆H₅— | —CH₂CH₂— | H | —CH₂— | 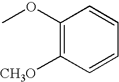 |
| E21 | O | C₆H₅— | —CH₂CH₂— | H | —CH₂CH₂— | 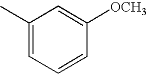 |
| E22 | O | C₆H₅— | —CH₂CH₂— | H | —CH₂CH₂— | 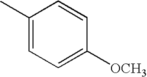 |
| E23 | O | C₆H₅— | —CH₂CH₂— | H | —CH₂CH₂— | 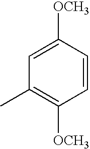 |
| E24 | O | C₆H₅— | —CH₂CH₂— | H | —CH(CH₃)CH₂— | 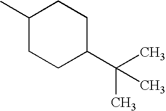 |
| E25 | O | C₆H₅— | —CH₂CH₂— | H | —CH(CH₃)CH₂— | 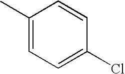 |
| E26 | O | C₆H₅— | —CH₂CH₂— | H | —CH₂CH₂CH₂— | 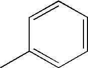 |
| E27 | O | C₆H₅— | —CH₂CH₂— | H | —CH₂CH₂CH₂— | 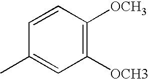 |
| E28 | O | C₆H₅— | —CH₂CH₂— | H | —CH₂— | 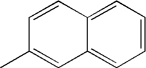 |
| E39 | O | C₆H₅— | —CH₂CH₂— | H | —CH(CH₃)— | 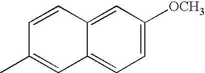 |
| E30 | O | C₆H₅— | —CH₂CH₂— | H | —CH₂— | 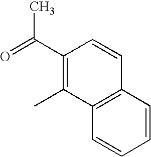 |
| E31 | O | C₆H₅— | —CH₂CH₂— | H | —CH₂— | 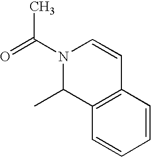 |
| E32 | O | C₆H₅— | —CH₂CH₂— | H | —CH₂— | 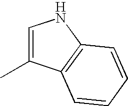 |
| E33 | O | C₆H₅— | —CH₂CH₂— | H | —CH₂CH₂— | 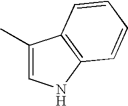 |
| E34 | O | C₆H₅— | —CH₂CH₂— | H | —CH₂— | 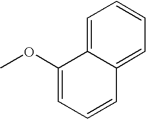 |
| E35 | O | C₆H₅— | —CH₂CH₂— | H | —CH₂CH(C₆H₅)— | 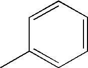 |
| E36 | O | C₆H₅— | —CH₂CH₂— | H | —CH₂— | 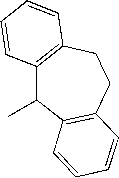 |
| E37 | O | C₆H₅— | —CH₂CH₂— | H | —CH₂— | 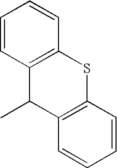 |
| E38 | O | C₆H₅— | —CH₂CH₂— | H | —CH₂— | 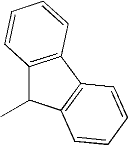 |
| E39 | O | C₆H₅— | —CH₂CH₂— | H | —CH(NHBOC)— | CH₃ |
| E40 | O | C₆H₅— | —CH₂CH₂— | H | —CH(NH₂)— | CH₃ |
| E41 | O | C₆H₅— | —CH₂CH₂— | H | —CH(NHBOC)CH₂— | 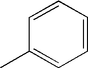 |
| E42 | O | C₆H₅— | —CH₂CH₂— | H | —CH(NH₂)CH₂ | 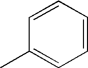 |
| E43 | O | C₆H₅— | —CH₂CH₂— | H | 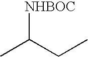 | 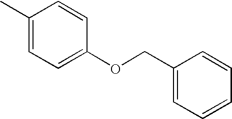 |
| E44 | O | C₆H₅— | —CH₂CH₂— | H | 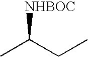 | 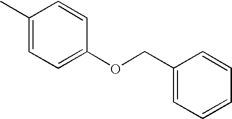 |
| E45 | O | C₆H₅— | —CH₂CH₂— | H | 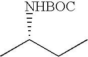 | 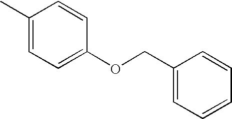 |
| E46 | O | C₆H₅— | —CH₂CH₂— | H | —CH(NH₂)CH₂— | 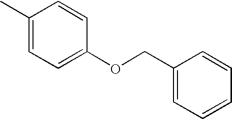 |
| E47 | O | C₆H₅— | —CH₂CH₂— | H | — | 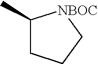 |
| E48 | O | C₆H₅— | —CH₂CH₂— | H | — | 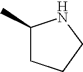 |
| E49 | O | C₆H₅— | —CH₂CH₂— | H | — | 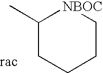 |
| E50 | O | C₆H₅— | —CH₂CH₂— | H | — | 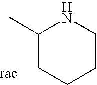 |
| E51 | O | C₆H₅— | —CH₂CH₂— | H | — | 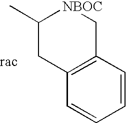 |
| E52 | O | C₆H₅— | —CH₂CH₂— | H | — | 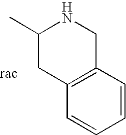 |
| F1 | O | C₆H₅— | —CH₂CH₂CH₂— | H | — | 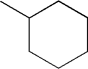 |
| F2 | O | C₆H₅— | —CH₂CH₂CH₂— | H | —CH₂CH₂CH₂— | 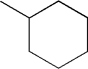 |
| F3 | O | C₆H₅— | —CH₂CH₂CH₂— | H | —CH(CH₃)CH₂— | 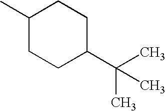 |
| F4 | O | C₆H₅— | —CH₂CH₂CH₂— | H | —CH₂— | 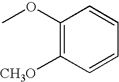 |
| F5 | O | C₆H₅— | —CH₂CH₂CH₂— | H | —CH₂— | 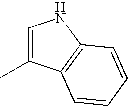 |
| F6 | O | C₆H₅— | —CH₂CH₂CH₂— | H | —CH₂CH(C₆H₅)— | 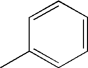 |
| F7 | O | C₆H₅— | —CH₂CH₂CH₂— | H | —CH₂— | 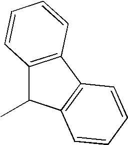 |
| G1 | O | 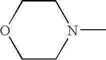 | —CH₂CH₂CH₂— | H | —CH₂CH₂— | —SCH₃ |
| G2 | O | 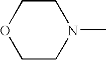 | —CH₂CH₂CH₂— | H | — | 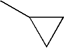 |
| G3 | O | 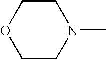 | —CH₂CH₂CH₂— | H | — | 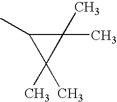 |
| G4 | O | 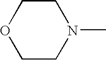 | —CH₂CH₂CH₂— | H | — | 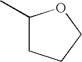 |
| G5 | O | 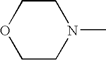 | —CH₂CH₂CH₂— | H | — | 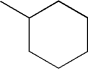 |
| G6 | O | 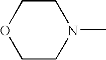 | —CH₂CH₂CH₂— | H | —CH₂— | 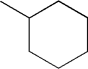 |
| G7 | O | 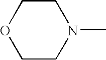 | —CH₂CH₂CH₂— | H | —CH₂CH₂CH₂— | 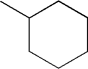 |
| G8 | O | 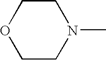 | —CH₂CH₂CH₂— | H | —CH₂— | 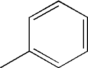 |
| G9 | O | 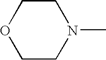 | —CH₂CH₂CH₂— | H | —CH(CH₃)— | 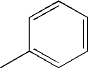 |
| G10 | O | 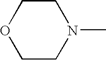 | —CH₂CH₂CH₂— | H | —CH₂— | 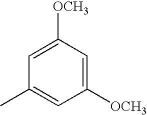 |
| G11 | O | 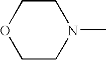 | —CH₂CH₂CH₂— | H | —CH₂— | 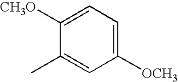 |
| G12 | O | 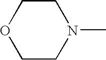 | —CH₂CH₂CH₂— | H | —CH₂— | 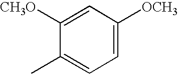 |
| G13 | O | 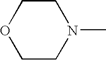 | —CH₂CH₂CH₂— | H | —CH₂— | 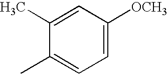 |
| G14 | O | 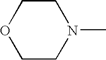 | —CH₂CH₂CH₂— | H | —CH₂CH₂— | 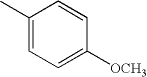 |
| G15 | O | 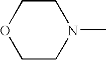 | —CH₂CH₂CH₂— | H | —CH₂CH(CH₃)— | 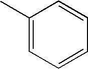 |
| G16 | O | 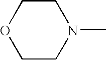 | —CH₂CH₂CH₂— | H | —CH₂CH₂— | 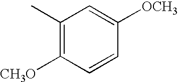 |
| G17 | O | 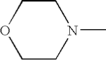 | —CH₂CH₂CH₂— | H | —CH₂CH₂— | 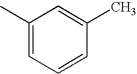 |
| G18 | O | 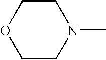 | —CH₂CH₂CH₂— | H | —CH₂CH₂— | 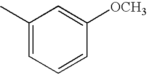 |
| G19 | O | 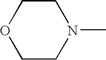 | —CH₂CH₂CH₂— | H | —CH₂— | 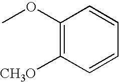 |
| G20 | O | 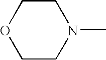 | —CH₂CH₂CH₂— | H | —CH₂— | 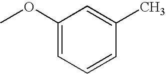 |
| G21 | O | 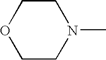 | —CH₂CH₂CH₂— | H | —CH₂— | 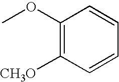 |
| G22 | O | 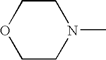 | —CH₂CH₂CH₂— | H | —CH₂— | 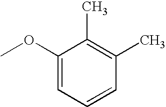 |
| G23 | O | 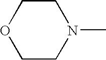 | —CH₂CH₂CH₂— | H | —CH₂CH₂CH₂— | 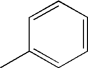 |
| G24 | O | 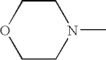 | —CH₂CH₂CH₂— | H | —CH₂— | 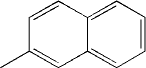 |
| G25 | O | 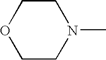 | —CH₂CH₂CH₂— | H | —CH₂— | 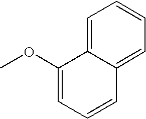 |
| G26 | O | 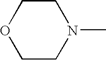 | —CH₂CH₂CH₂— | H | —CH₂— | 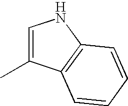 |
| G27 | O | 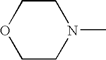 | —CH₂CH₂CH₂— | H | —CH₂CH₂— | 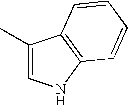 |
| G28 | O | 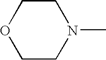 | —CH₂CH₂CH₂— | H | —CH₂— | 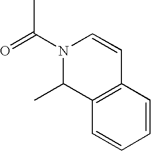 |
| G29 | O | 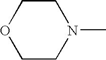 | —CH₂CH₂CH₂— | H | —CH₂CH(C₆H₅)— | 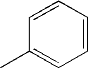 |
| G30 | O | 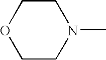 | —CH₂CH₂CH₂— | H | —CH₂— | 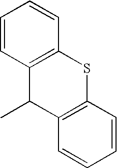 |
| H1 | O | 4-benzyl-oxophenyl | —CH₂CH₂— | H | —CH₂CH₂CH₂— | 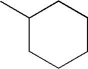 |
| I1 | O | C₆H₅— | —CH₂CH₂— | CH₃ | — | 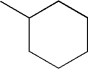 |
| I2 | O | C₆H₅— | —CH₂CH₂— | CH₃ | —CH₂CH₂CH₂— | 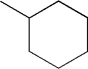 |
| I3 | O | C₆H₅— | —CH₂CH₂— | CH₃ | —CH(CH₃)CH₂— | 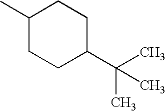 |
| I4 | O | C₆H₅— | —CH₂CH₂— | CH₃ | —CH₂— | 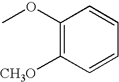 |
| I5 | O | C₆H₅— | —CH₂CH₂— | CH₃ | —CH₂— | 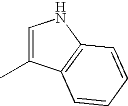 |
| I6 | O | C₆H₅— | —CH₂CH₂— | CH₃ | —CH₂CH(C₆H₅)— | 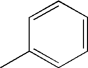 |
| I7 | O | C₆H₅— | —CH₂CH₂— | CH₃ | —CH₂— | 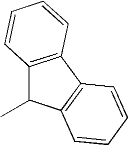 |
| J1 | O | C₆H₅— | —CH₂CH₂— | C₆H₅ | — | 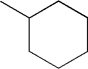 |
| J2 | O | C₆H₅— | —CH₂CH₂— | C₆H₅ | —CH₂— | 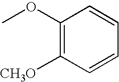 |
| J3 | O | C₆H₅— | —CH₂CH₂— | C₆H₅ | —CH₂— | 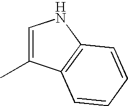 |
| J4 | O | C₆H₅— | —CH₂CH₂— | C₆H₅ | —CH₂CH(C₆H₅)— | 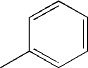 |
| J5 | O | C₆H₅— | —CH₂CH₂— | C₆H₅ | —CH₂— | 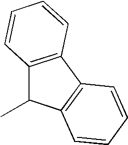 |
| K1 | NH | C₆H₅— | —CH₂CH₂— | H | —CH₂CH₂— | —SCH₃ |
| K2 | NH | C₆H₅— | —CH₂CH₂— | H | — | 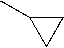 |
| K3 | NH | C₆H₅— | —CH₂CH₂— | H | — | 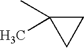 |
| K4 | NH | C₆H₅— | —CH₂CH₂— | H | — | 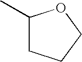 |
| K5 | NH | C₆H₅— | —CH₂CH₂— | H | —CH₂CH₂CH₂— | 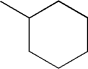 |
| K6 | NH | C₆H₅— | —CH₂CH₂— | H | — | 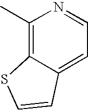 |
| K7 | NH | C₆H₅— | —CH₂CH₂— | H | — | 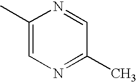 |
| K8 | NH | C₆H₅— | —CH₂CH₂— | H | — | 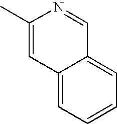 |
| K9 | NH | C₆H₅— | —CH₂CH₂— | H | — | 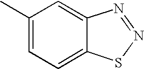 |
| K10 | NH | C₆H₅— | —CH₂CH₂— | H | — | 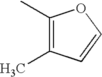 |
| K11 | NH | C₆H₅— | —CH₂CH₂— | H | — | 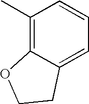 |
| K12 | NH | C₆H₅— | —CH₂CH₂— | H | — | 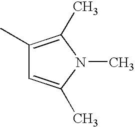 |
| K13 | NH | C₆H₅— | —CH₂CH₂— | H | —CH₂— | 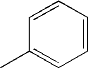 |
| K14 | NH | C₆H₅— | —CH₂CH₂— | H | —CH₂— | 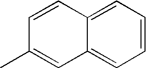 |
| K15 | NH | C₆H₅— | —CH₂CH₂— | H | —CH₂— | 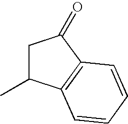 |
| K16 | NH | C₆H₅— | —CH₂CH₂— | H | —CH₂— | 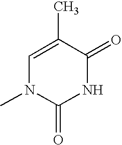 |
| K17 | NH | C₆H₅— | —CH₂CH₂— | H | —CH(CH₃)— | 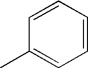 |
| K18 | NH | C₆H₅— | —CH₂CH₂— | H | —CH(CH₃)— | 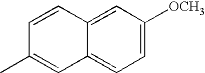 |
| K19 | NH | C₆H₅— | —CH₂CH₂— | H | —CH₂CH₂— | 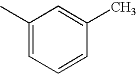 |
| K20 | NH | C₆H₅— | —CH₂CH₂— | H | —CH₂CH₂— | 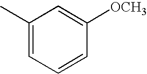 |
| K21 | NH | C₆H₅— | —CH₂CH₂— | H | —CH₂CH₂— | 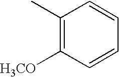 |
| K22 | NH | C₆H₅— | —CH₂CH₂— | H | —CH₂CH₂— | 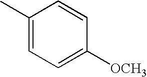 |
| K23 | NH | C₆H₅— | —CH₂CH₂— | H | —CH(CH₃)CH₂— | 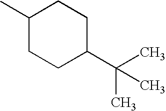 |
| K24 | NH | C₆H₅— | —CH₂CH₂— | H | —CH₂— | 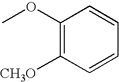 |
| K25 | NH | C₆H₅— | —CH₂CH₂— | H | —CH₂CH₂CH₂— | 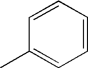 |
| K26 | NH | C₆H₅— | —CH₂CH₂— | H | —CH₂CH₂CH₂— | 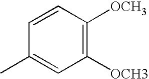 |
| K27 | NH | C₆H₅— | —CH₂CH₂— | H | —CH₂— | —NHCOCH₃ |
| K28 | NH | C₆H₅— | —CH₂CH₂— | H | —CHNHCOCH₃ | —SCH₃ |
| | | | | | —CH₂CH₂— | |
| K29 | NH | C₆H₅— | —CH₂CH₂— | H | —CH₂— | 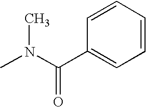 |
| K30 | NH | C₆H₅— | —CH₂CH₂— | H | —CH₂— | 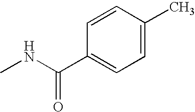 |
| K31 | NH | C₆H₅— | —CH₂CH₂— | H | —CH₂— | 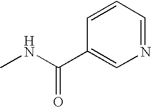 |
| K32 | NH | C₆H₅— | —CH₂CH₂— | H | —CH(CH₃)— | 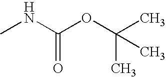 |
| K33 | NH | C₆H₅— | —CH₂CH₂— | H | —CH(CH₂C₆H₅)— | 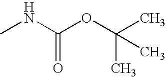 |
| K34 | NH | C₆H₅— | —CH₂CH₂— | H | — | 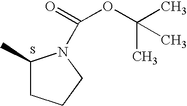 |
| K35 | NH | C₆H₅— | —CH₂CH₂— | H | — | 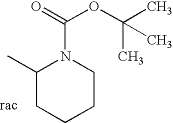 |
| K36 | NH | C₆H₅— | —CH₂CH₂— | H | — | 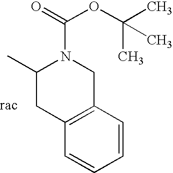 |
| K37 | NH | C₆H₅— | —CH₂CH₂— | H | —CH(NHBOC)CH₂— | 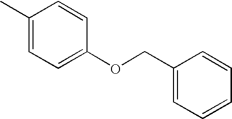 |
| K38 | NH | C₆H₅— | —CH₂CH₂— | H | —CH(NH₂)CH₂— | 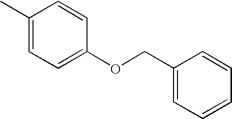 |
| K39 | NH | C₆H₅— | —CH₂CH₂— | H | —CH₂— | 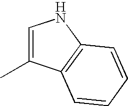 |
| K40 | NH | C₆H₅— | —CH₂CH₂— | H | —CH₂— | 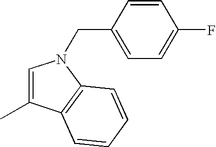 |
| K41 | NH | C₆H₅— | —CH₂CH₂— | H | —CH₂— | 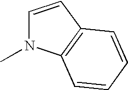 |
| K42 | NH | C₆H₅— | —CH₂CH₂— | H | —CH₂CH₂— | 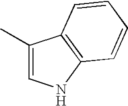 |
| K43 | NH | C₆H₅— | —CH₂CH₂— | H | —CH₂— | 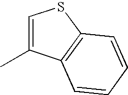 |
| K44 | NH | C₆H₅— | —CH₂CH₂— | H | —CH₂CH(C₆H₅)— | —C₆H₅ |
| K45 | NH | C₆H₅— | —CH₂CH₂— | H | —CH(C₆H₅)CH₂— | —C₆H₅ |
| K46 | NH | C₆H₅— | —CH₂CH₂— | H | —CH₂— | 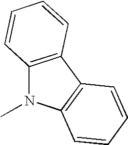 |

Still yet in another embodiment the present invention provides the compound of formula I, which is
Rac-4-hydroxy-5-isobutyl-3-[(9H-thioxanthen-9-yl)-acetyl]-5H-furan-2-one;
3-[3-(4-tert-Butyl-phenyl)-2(R,S)-methyl-propionyl]-5 (R,S)-cyclohexylmethyl-4-hydroxy-5H-furan-2-one;
5-Chloro-N-(2-{4-[3-(5 (R,S)-cyclohexylmethyl-4-hydroxy-2-oxo-2,5-dihydro-furan-3-yl)-2(R,S)-methyl-3-oxo-propyl]-phenyl}-ethyl)-2-methoxy-benzamide;
Rac-5-cyclohexylmethyl-4-hydroxy-3-[(1H-indol-3-yl)-acetyl]-5H-furan-2-one;
Rac-5-cyclohexylmethyl-3-{[1-(4-fluoro-benzyl)-1H-indol-3-yl]-acetyl}-4-hydroxy-5H-furan-2-one;
Rac-5-cyclohexylmethyl-3-[(9H-fluoren-9-yl)-acetyl]-4-hydroxy-5H-furan-2-one;
Rac-3-(carbazol-9-yl-acetyl)-5-cyclohexylmethyl-4-hydroxy-5H-furan-2-one;
5 (R,S)-Benzyl-3-[3-(4-tert-butyl-phenyl)-2(R,S)-methyl-propionyl]-4-hydroxy-5H-furan-2-one;
Rac-4-hydroxy-3-[(2-methoxy-phenoxy)-acetyl]-5-phenethyl-5H-furan-2-one;
Rac-4-hydroxy-3-[(1H-indol-3-yl)-acetyl]-5-phenethyl-5H-furan-2-one;
Rac-3-(3,3-diphenyl-propionyl)-4-hydroxy-5-phenethyl-5H-furan-2-one;
Rac-4-hydroxy-3-[(1H-indol-3-yl)-acetyl]-5-(3-phenyl-propyl)-5H-furan-2-one;
Rac-3-[(9H-fluoren-9-yl)-acetyl]-4-hydroxy-5-methyl-5-phenethyl-5H-furan-2-one;
4-Hydroxy-3 (R,S)-[2-(6-methoxy-naphthalen-2-yl)-propionyl]-5 (R,S)-phenethyl-1,5-dihydro-pyrrol-2-one;
[1-(4-Benzyloxy-benzyl)-2-(4-hydroxy-2-oxo-5 (R,S)-phenethyl-2,5-dihydro-1H-pyrrol-3-yl)-2(R,S)-oxo-ethyl]-carbamic acid tert-butyl ester;
Rac-4-hydroxy-3-(indol-1-yl-acetyl)-5-phenethyl-1,5-dihydro-pyrrol-2-one; or
Rac-3-(carbazol-9-yl-acetyl)-4-hydroxy-5-phenethyl-1,5-dihydro-pyrrol-2-one.

The present compounds of formula I and their pharmaceutically acceptable salts can be prepared by methods known in the art, for example, by the process described below, which process comprises

acylation of a compound of formula II

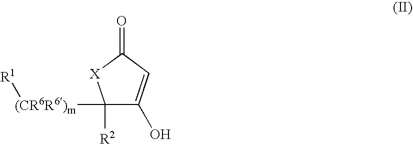

wherein

### X is O or NH;

R¹ is lower alkyl, cycloalkyl, heterocycloalkyl or aryl, wherein the aryl ring is unsubstituted or substituted by benzyloxy;
R² is H, lower alkyl or aryl;
R⁶ and R^{6′} are each independently selected from H, lower alkyl or —SCH₃;
m is 1, 2 or 3; with a carboxylic acid of formula III

HOOC—(CHR⁴)ₙ—(CR⁵R^{5′})ₚ—R³ (III)

wherein
R³ is lower alkyl, —SCH₃, acetyl,

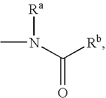

wherein R^{a} is H or lower alkyl, R^{b} is lower alkyl, heteroaryl, —OC(CH₃)₃ or aryl, wherein the aryl ring is unsubstituted or substituted by lower alkyl,

cycloalkyl, wherein the cycloalkyl ring is unsubstituted or substituted by lower alkyl or aryl,

heterocycloalkyl, wherein the heterocycloalkyl ring is unsubstituted or substituted by —COOC(CH₃)₃, or

(CH═CR′)ₒ-aryl, wherein the aryl ring is unsubstituted or substituted by lower alkyl, alkoxy, hydroxyl, benzyloxy, halogen, acetyl, —(CH₂)₂NHSO₂Ph, —NHCO(CH₂)₂NHCOOC(CH₃)₃, —(CH₂)₂NHCOC₆H₃OCH₃Cl, or for the non aromatic part of fused ring system also by oxo,

o is 0 or 1;

### R′ is H,

lower alkyl,

aryloxy, wherein the aryl ring is unsubstituted or substituted by lower alkyl or alkoxy, or

(CH═CH)_{q}-heteroaryl, wherein the heteroaryl ring is unsubstituted or substituted by lower alkyl, acetyl, alkoxy, halogen, —COOC(CH₃)₃ or by halogen substituted benzyl; or for the non aromatic part of fused ring system also by oxo;

q is 0 or 1;
R⁴ is H, lower alkyl, —(CH₂)₂SCH₃, —NHCOCH₃, —NHSO₂p-Cl-Ph, amino, —NHCOOC(CH₃)₃, hydroxyl, aryl, benzyl or halogen substituted benzyl;
R⁵ and R^{5′} are each independently selected from H, lower alkyl or aryl;
n is 0 or 1; and
p is 0, 1, 2 or 3;
to produce a compound of formula I

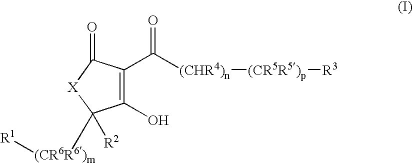

wherein X, R¹, R², R³, R⁴, R⁵, R^{5′}, R⁶, R^{6′}, m, n and p, are as defined above, and if desired, converting the compounds obtained into pharmaceutically acceptable acid addition salts.

The compounds of formula Ia may be prepared in accordance with the following scheme 1:

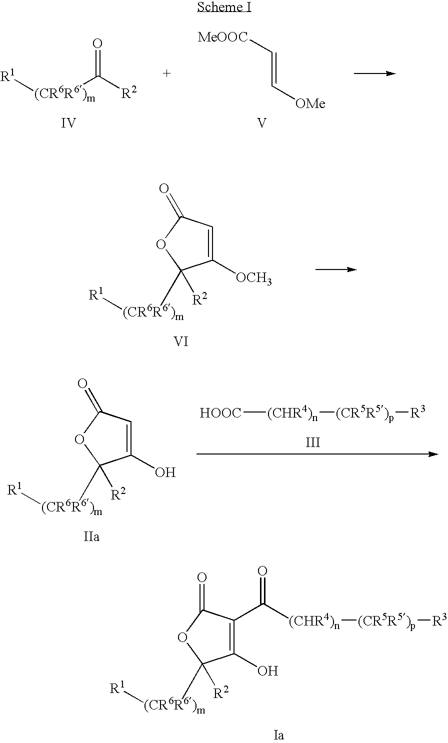

Aldehydes or ketones IV may be reacted with 3(E)-methoxy-acrylic acid methyl ester V (Miyata, Okiko; Schmidt, Richard R.; Angewandte Chemie (1982), 94(8), 651-2) in solvents like diethyl ether or THF in the presence of a base like lithiumdiisopropylamide (LDA) at a temperature in the range of −100° C. to −50° C., or at −80° C. to give the tetronic acid derivatives VI.

Cleavage of the methoxy group in VI may be accomplished with a strong mineral acid such as HI, HBr or HCl preferably HBr in water and acetic acid at a temperature in the range of 20° C. to 100° C., or at 40° C. to give the tetronic acid IIa.

Acylation of IIa followed by Fries rearrangement (Nomura, Keiichi; Hori, Kozo; Arai, Mikio; Yoshii, Eiichi; Chem. Pharm. Bull. (1986), 34(12), 5188-90) may be effected with a carboxylic acid and a dehydrating agent such as dicyclohexyl carbodiimide (DCC) or N-(3-dimethylaminopropyl)-N′-ethyl carbodiimide hydrochloride (EDC), preferably EDC and a base like an alkylamine, preferably NEt₃ in a solvent like CH₂Cl₂ or THF, preferably THF in the presence of 10 to 50 mole %, preferably 30 mole % of 4-dimethylamino pyridine (DMAP) at a temperature in the range of 0° C. to 35° C., preferably at 25° C. to give the acylated tetronic acid Ia.

The compounds of formula Ib may be prepared in accordance with the following scheme 2:

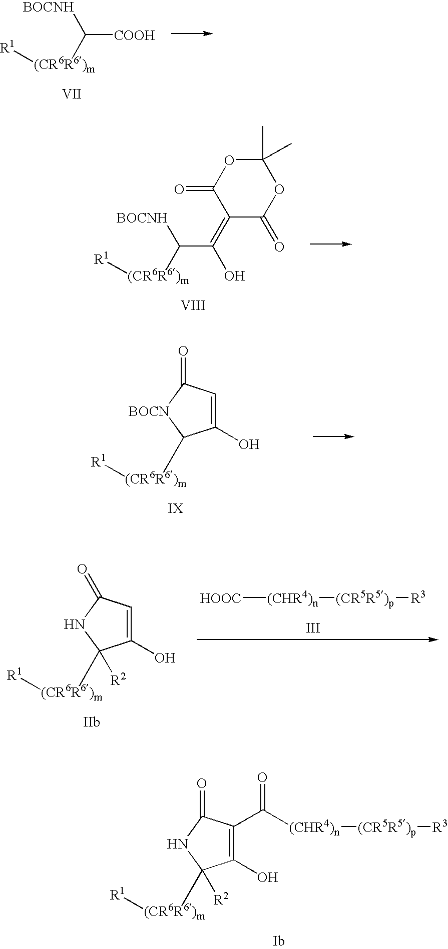

The tetramic acid IIb may be prepared according to the method described by Jouin, P; Castro, B; J. Chem. Soc. Perkin Trans. I, 1987, 1177.

Acylation of IIb followed by Fries rearrangement (Nomura, Keiichi; Hori, Kozo; Arai, Mikio; Yoshii, Eiichi; Chem. Pharm. Bull. (1986), 34(12), 5188-90) may be effected with a carboxylic acid and a dehydrating agent such as DCC or EDC, preferably EDC and a base like an alkylamine, preferable NEt₃ in a solvent like CH₂Cl₂ or THF, preferably THF in the presence of 10 to 50 mole %, preferably 30 mole % of DMAP at temperatures between 0° C. to 35° C., preferably 25° C. to give the acylated tetramic acid Ib.

A more detailed description for preparing a compound of formula I can be found in Examples A1-A46, B1-B39, C1-C33, D1-D7, E1-E52, F1-F7, G1-G30, H1, I1-I7, J1-J5 and K1-K46.

The compounds of formula I and their pharmaceutically acceptable salts possess valuable pharmacological properties. Specifically, it has been found that the compounds of the present invention inhibit the β-secretase.

Cellular screening methods for inhibitors of A-beta production, testing methods for the in vivo suppression of A-beta production, and assays with membranes or cellular extracts for the detection of secretase activity are known in the art and have been disclosed in numerous publications, including WO 98/22493, U.S. Pat. No. 5,703,129, U.S. Pat. No. 5,593,846 and GB 2,395,124; all hereby incorporated by reference. β-Secretase has been described in several publications including EP 855,444, WO 00/17,369, WO 00/58,479, WO 00/47,618, WO 01/00,663 and WO 01/00,665.

For example, inhibition of β-secretase of the pharmaceutical compounds may be demonstrated by their ability, e.g., to inhibit the cleavage of a fluorescent peptide substrate (e.g. in an assay like e.g. the FRET Assay as described inter alia by Grueninger-Leitch et al.) or to displace, e.g., a peptidic β-secretase inhibitor at the active binding site of β-secretase, e.g. as demonstrated in accordance with the following test method.

### Competitive Radioligand Binding Assay (RLBA)

96 well microplates (Optiplate Packard) are coated with purified BACE protein (see e.g. GB 2,385,124: Examples 1 and 2) using a concentration of 1 μg/ml in 30 mM sodium citrate buffer adjusted to pH 5.5. The coating is achieved by incubation of 100 μl/well for 1-3 days at 4° C. The plate is then washed with 2×300 μl/well of 10 mM citrate pH 4.1. To each well 100 μl binding buffer (30 mM citrate, 100 mM NaCl, 0.1% BSA, pH 4.1) is dispensed. The test compound is added in 5 μl from a DMSO stock solution or appropriate dilutions. To this the tracer (tritiated Compound A, see e.g. GB 2,385,124: Example 4) is added in 10 μl/well from a 10 μCi/ml stock solution in binding buffer. After incubation for 1.5-2 hours in a humid chamber at ambient temperature the plate is washed with 2×300 μl/well water and flipped on a dry towel. Following the addition of 50 μl/well MicroScint20 (Packard) the plate is sealed and vibrated for 5 seconds. The bound radioactivity is counted on a Topcount (Packard). Total binding is typically between 2000 and 10000 cpm/well depending mainly on the purity and concentration of the BACE protein. Non-specific binding as assessed by competition with >1 μM peptidic inhibitor (Bachem #H-4848) is typically between 30 and 300 cpm/well. The IC-50 values are calculated by Microsoft Excel FIT.

Some exemplary IC₅₀ inhibition data for the β-secretase inhibition are given in Table 2 below:

TABLE 2

| Example No. | IC₅₀ in vitro (μM) |
|---|---|
| C12 | 12 |
| C9 | 13 |
| C19 | 15 |
| D2 | 33 |
| E7 | 57 |
| F5 | 14 |
| G29 | 85 |
| C33 | 11 |
| I7 | 31 |
| J4 | 41 |
| K38 | 16 |
| K46 | 36 |

In another embodiment, the present invention provides pharmaceutical compositions containing compounds of the invention or a pharmaceutically acceptable salt thereof and a pharmaceutically acceptable carrier. Such compositions can be in the form of tablets, dragées, hard and soft gelatine capsules, solutions, emulsions or suspensions. The pharmaceutical compositions also can be in the form of suppositories or injectable solutions.

The pharmaceutical compositions of the invention, in addition to one or more compounds of the invention, contain a pharmaceutically acceptable carrier. Suitable pharmaceutically acceptable carriers include pharmaceutically inert, inorganic or organic carriers. Lactose, corn starch or derivatives thereof, talc, stearic acids or its salts and the like can be used, for example, as such carriers for tablets, coated tablets, dragées and hard gelatine capsules. Suitable carriers for soft gelatine capsules are, for example, vegetable oils, waxes, fats, semi-solid and liquid polyols and the like. Depending on the nature of the active substance no carriers are, however, usually required in the case of soft gelatine capsules. Suitable carriers for the production of solutions and syrups are, for example, water, polyols, glycerol, vegetable oil and the like. Suitable carriers for suppositories are, for example, natural or hardened oils, waxes, fats, semi-liquid or liquid polyols and the like.

In addition, the pharmaceutical compositions can, moreover, contain preservatives, solubilizers, stabilizers, wetting agents, emulsifiers, sweeteners, colorants, flavorants, salts for varying the osmotic pressure, buffers, masking agents or antioxidants. They can also contain still other therapeutically valuable substances.

The invention also provides a process for the manufacture of compositions of the invention. Such process comprises bringing one or more compounds of the invention and/or a pharmaceutically acceptable acid addition salt thereof and, if desired, one or more other therapeutically valuable substances into a galenical administration form together with one or more therapeutically inert carriers.

The pharmaceutical compositions can be administered in a conventional manner, for example, orally rectally, or parenterally. The compositions can be administered orally, e.g. in the form of tablets, coated tablets, dragées, hard and soft gelatine capsules, solutions, emulsions or suspensions. The administration can, however, also be effected rectally, e.g. in the form of suppositories, parenterally, e.g. in the form of injectable solutions.

Compounds of the invention have β-secretase inhibitory activity. Therefore, they are useful for the treatment of diseases for which inhibition of β-secretase is desirable. For example, the compounds of the invention are useful for the treatment of CNS diseases, such as Alzheimer's disease. In one embodiment, the present invention provides a method for treating Alzheimer's disease which comprises administering a therapeutically effective amount of a compound of the invention, for example, a compound of formula I or a pharmaceutically acceptable salt thereof.

The dosage at which a compound of the invention is administered can vary within wide limits and will, of course, have to be adjusted to the individual requirements in each particular case. In the case of oral administration the dosage for adults can vary from about 0.01 mg to about 1000 mg per day of a compound of general formula I or of the corresponding amount of a pharmaceutically acceptable salt thereof. The daily dosage may be administered as single dose or in divided doses and, in addition, the upper limit can also be exceeded when this is found to be indicated.

Tablet Formulation (Wet Granulation)

| | | mg/tablet | | | |
|---|---|---|---|---|---|
| Item | Ingredients | 5 mg | 25 mg | 100 mg | 500 mg |
| 1. | Compound of formula I | 5 | 25 | 100 | 500 |
| 2. | Lactose Anhydrous DTG | 125 | 105 | 30 | 150 |
| 3. | Sta-Rx 1500 | 6 | 6 | 6 | 30 |
| 4. | Macrocrystalline Cellulose | 30 | 30 | 30 | 150 |
| 5. | Magnesium Stearate | 1 | 1 | 1 | 1 |
| | Total | 167 | 167 | 167 | 831 |

### Manufacturing Procedure

1. Mix items 1, 2, 3 and 4 and granulate with purified water.
2. Dry the granules at 50° C.
3. Pass the granules through suitable milling equipment.
4. Add item 5 and mix for three minutes; compress on a suitable press.

Capsule Formulation

| | | mg/capsule | | | |
|---|---|---|---|---|---|
| Item | Ingredients | 5 mg | 25 mg | 100 mg | 500 mg |
| 1. | Compound of formula I | 5 | 25 | 100 | 500 |
| 2. | Hydrous Lactose | 159 | 123 | 148 | — |
| 3. | Corn Starch | 25 | 35 | 40 | 70 |
| 4. | Talc | 10 | 15 | 10 | 25 |
| 5. | Magnesium Stearate | 1 | 2 | 2 | 5 |
| | Total | 200 | 200 | 300 | 600 |

### Manufacturing Procedure

1. Mix items 1, 2 and 3 in a suitable mixer for 30 minutes.
2. Add items 4 and 5 and mix for 3 minutes.
3. Fill into a suitable capsule.

### EXAMPLE A1

### (RS)-4-Hydroxy-5-isobutyl-3-(3-methyl-butyryl)-5H-furan-2-one

### a) 5-Isobutyl-4-methoxy-5H-furan-2-one

To a solution of 20 ml of LDA (2M in THF) and 130 ml of THF was added at −95° C. to −100° C. a solution of 5.47 g of 3(E)-methoxy-acrylic acid methyl ester in 4.5 ml of THF within 1 min, stirring was continued at the same temperature for 5 min, which was followed by the addition of a pre-cooled (−78° C.) solution of 33 mmole of the 3-methyl butyraldehyde in 4.5 ml of THF within 2 min and stirring was continued at −100° C. for 30 min and at −78° C. for 1 h. The cold solution was poured onto 130 ml of ice-water, the pH was adjusted to 4 with 6.5 ml of aqueous HCl (37%) and the layers were separated. The aqueous layer was extracted twice with dichloromethane, the organic layers were washed with brine, dried and evaporated. The residue was chromatographed on silica (n-heptane/AcOEt, various ratios) to give the 5-isobutyl-4-methoxy-5H-furan-2-one in 30-40% yield.

MS: 171.2 (M+H)+

### b) 4-Hydroxy-5-isobutyl-5H-furan-2-one

A mixture of the 5-isobutyl-4-methoxy-5H-furan-2-one (10 mmole) and 15 ml of aqueous HCl (37%) was stirred at 40° C. until completion of the reaction. The suspension was filtered and the residue washed with ice-cold water and dried. An oily reaction mixture was extracted with dichloromethane, the organic layers were washed with brine, dried and evaporated. The residue was either triturated with AcOEt/hexane or chromatographed with dichloromethane/MeOH (various ratios) to give the 4-hydroxy-5-isobutyl-5H-furan-2-one in 60-90% yield.

MS: 100.1 (M−C₄H₈)⁺

### c) (RS)-4-Hydroxy-5-isobutyl-3-(3-methyl-butyryl)-5H-furan-2-one

To as suspension of the 4-hydroxy-5-isobutyl-5H-furan-2-one (0.2 mmole), NEt₃ (0.68 mmole), DMAP (0.066 mmole) and EDC (0.44 mmole) in 2 ml of THF was added at 22° C. 3-methyl-butyric acid (0.22 mmole) (commercially available) and stirring was continued until completion of the reaction. The pH of the reaction mixture was adjusted to 3 using aqueous HCl (2 N), the aqueous solution was saturated with NaCl, the organic layer was separated, washed with brine dried and evaporated. The residue was purified on preparative HPLC (RP-18, CH₃CN/H₂O, gradient) to give the (RS)-4-hydroxy-5-isobutyl-3-(3-methyl-butyryl)-5H-furan-2-one in 10-60% yield.

MS m/e (%): 239.2 (M−H)⁻

### EXAMPLE A2

### 4-Hydroxy-5-isobutyl-3-(3-methylsulfanyl-propionyl)-5H-furan-2-one

The title compound was obtained in comparable yields according to the procedures described for example A1 using 3-methylsulfanyl-propionic acid (commercially available) instead of 3-methyl-butyric acid in step c).

MS: 256.9 (M−H)⁻

### EXAMPLE A3

### 4-Hydroxy-5-isobutyl-3-(4-methyl-pentanoyl)-5H-furan-2-one

The title compound was obtained in comparable yields according to the procedures described for example A1 using 4-methyl-pentanoic acid (commercially available) instead of 3-methyl-butyric acid in step c).

MS: 253.2 (M−H)⁻

### EXAMPLE A4

### 1-(4-Hydroxy-5-isobutyl-2-oxo-2,5-dihydro-furan-3-yl)-2-methyl-pentane-1,4-dione

The title compound was obtained in comparable yields according to the procedures described for example A1 using 2-methyl-4-oxo-pentanoic acid (commercially available) instead of 3-methyl-butyric acid in step c).

MS: 268.3 (M−H)⁻

### EXAMPLE A5

### 4-Hydroxy-5-isobutyl-3-(2,2,3,3-tetramethyl-cyclopropanecarbonyl)-5H-furan-2-one

The title compound was obtained in comparable yields according to the procedures described for example A1 using 2,2,3,3-tetramethyl-cyclopropanecarboxylic acid (commercially available) instead of 3-methyl-butyric acid in step c).

MS: 279.0 (M−H)⁻

### EXAMPLE A6

### 4-Hydroxy-5-isobutyl-3-(tetrahydro-furan-2-carbonyl)-5H-furan-2-one

The title compound was obtained in comparable yields according to the procedures described for example A1 using tetrahydro-furan-2-carboxylic acid (commercially available) instead of 3-methyl-butyric acid in step c).

MS: 252.9 (M−H)⁻

### EXAMPLE A7

### 3-Cyclohexanecarbonyl-4-hydroxy-5-isobutyl-5H-furan-2-one

The title compound was obtained in comparable yields according to the procedures described for example A1 using cyclohexanecarboxylic acid (commercially available) instead of 3-methyl-butyric acid in step c).

MS: 265.2 (M−H)⁻

### EXAMPLE A8

### 3-(4-tert-Butyl-cyclohexanecarbonyl)-4-hydroxy-5-isobutyl-5H-furan-2-one

The title compound was obtained in comparable yields according to the procedures described for example A1 using 4-tert-butyl-cyclohexanecarboxylic acid (commercially available) instead of 3-methyl-butyric acid in step c).

MS: 321.1 (M−H)⁻

### EXAMPLE A9

### 3-(Cyclopent-2-enyl-acetyl)-4-hydroxy-5-isobutyl-5H-furan-2-one

The title compound was obtained in comparable yields according to the procedures described for example A1 using cyclopent-2-enecarboxylic acid (prepared according to Palaty, Jan; Abbott, Frank S.; Journal of Medicinal Chemistry (1995), 38(17), 3398-406) instead of 3-methyl-butyric acid in step c).

MS: 263.1 (M−H)⁻

### EXAMPLE A10

### 3-(2-Cyclohexyl-acetyl)-4-hydroxy-5-isobutyl-5H-furan-2-one

The title compound was obtained in comparable yields according to the procedures described for example A1 using cyclohexyl-acetic acid (commercially available) instead of 3-methyl-butyric acid in step c).

MS: 281.1 (M+H)⁺

### EXAMPLE A11

### 3-(4-Cyclohexyl-butyryl)-4-hydroxy-5-isobutyl-5H-furan-2-one

The title compound was obtained in comparable yields according to the procedures described for example A1 using cyclohexyl-butyric acid (commercially available) instead of 3-methyl-butyric acid in step c).

MS: 307.0 (M−H)⁻

### EXAMPLE A12

### 4-Hydroxy-5-isobutyl-3-(2-phenoxy-benzoyl)-5H-furan-2-one

The title compound was obtained in comparable yields according to the procedures described for example A1 using 2-phenoxy-benzoic acid (commercially available) instead of 3-methyl-butyric acid in step c).

MS: 351.2 (M−H)⁻

### EXAMPLE A13

### 4-Hydroxy-5-isobutyl-3-phenylacetyl-5H-furan-2-one

The title compound was obtained in comparable yields according to the procedures described for example A1 using phenyl-acetic acid (commercially available) instead of 3-methyl-butyric acid in step c).

MS: 275.1 (M+H)⁺

### EXAMPLE A14

### 4-Hydroxy-5-isobutyl-3-o-tolylacetyl-5H-furan-2-one

The title compound was obtained in comparable yields according to the procedures described for example A1 using o-tolyl-acetic acid (commercially available) instead of 3-methyl-butyric acid in step c).

MS: 287.2 (M−H)⁻

### EXAMPLE A15

### 3-[(4-Chloro-phenyl)-acetyl]-4-hydroxy-5-isobutyl-5H-furan-2-one

The title compound was obtained in comparable yields according to the procedures described for example A1 using (4-chloro-phenyl)-acetic acid (commercially available) instead of 3-methyl-butyric acid in step c).

MS: 307.2 (M−H)⁻

### EXAMPLE A16

### 4-Hydroxy-5-isobutyl-3-[2-(4-methoxy-3-methyl-phenyl)-acetyl]-5H-furan-2-one

The title compound was obtained in comparable yields according to the procedures described for example A1 using (4-methoxy-3-methyl-phenyl)-acetic acid (commercially available) instead of 3-methyl-butyric acid in step c).

MS: 317.1 (M−H)⁻

### EXAMPLE A17

### 3-[2-(3,5-Dimethoxy-phenyl)-acetyl]-4-hydroxy-5-isobutyl-5H-furan-2-one

The title compound was obtained in comparable yields according to the procedures described for example A1 using (3,5-dimethoxy-phenyl)-acetic acid (commercially available) instead of 3-methyl-butyric acid in step c).

MS: 352.3 (M+NH₄)⁺

### EXAMPLE A18

### 3-[2-(2,5-Dimethoxy-phenyl)-acetyl]-4-hydroxy-5-isobutyl-5H-furan-2-one

The title compound was obtained in comparable yields according to the procedures described for example A1 using (2,5-dimethoxy-phenyl)-acetic acid (commercially available) instead of 3-methyl-butyric acid in step c).

MS: 335.2 (M+H)⁺

### EXAMPLE A19

### 3-[2-(2,4-Dimethoxy-phenyl)-acetyl]-4-hydroxy-5-isobutyl-5H-furan-2-one

The title compound was obtained in comparable yields according to the procedures described for example A1 using (3,4-dimethoxy-phenyl)-acetic acid (commercially available) instead of 3-methyl-butyric acid in step c).

MS: 335.2 (M+H)⁺

### EXAMPLE A20

### 3-(2-phenyl-propionyl-4-hydroxy-5-isobutyl-5H-furan-2-one

The title compound was obtained in comparable yields according to the procedures described for example A1 using 2-phenyl-propionic acid (commercially available) instead of 3-methyl-butyric acid in step c).

MS: 287.0 (M−H)⁻

### EXAMPLE A21

### 4-Hydroxy-5-isobutyl-3-(2-phenyl-butyryl)-5H-furan-2-one

The title compound was obtained in comparable yields according to the procedures described for example A1 using 2-phenyl-butyric acid (commercially available) instead of 3-methyl-butyric acid in step c).

MS: 303.2 (M+H)⁺

### EXAMPLE A22

### 4-Hydroxy-5-isobutyl-3-[2-(6-methoxy-naphthalen-2-yl)-propionyl]-5H-furan-2-one

The title compound was obtained in comparable yields according to the procedures described for example A1 using 2-(6-methoxy-naphthalen-2-yl)-propionic acid (commercially available) instead of 3-methyl-butyric acid in step c).

MS: 369.2 (M+H)⁺

### EXAMPLE A23

### 4-Hydroxy-5-isobutyl-3-(3-phenyl-propionyl)-5H-furan-2-one

The title compound was obtained in comparable yields according to the procedures described for example A1 using 3-phenyl-propionic acid (commercially available) instead of 3-methyl-butyric acid in step c).

MS: 287.0 (M+H)⁻

### EXAMPLE A24

### 4-Hydroxy-5-isobutyl-3-(3-m-tolyl-propionyl)-5H-furan-2-one

The title compound was obtained in comparable yields according to the procedures described for example A1 using 3-m-tolyl-propionic acid (commercially available) instead of 3-methyl-butyric acid in step c).

MS: 320.4 (M+NH₄)⁺

### EXAMPLE A25

### 4-Hydroxy-5-isobutyl-3-[3-(3-methoxy-phenyl)-propionyl]-5H-furan-2-one

The title compound was obtained in comparable yields according to the procedures described for example A1 using 3-(3-methoxy-phenyl)-propionic acid (commercially available) instead of 3-methyl-butyric acid in step c).

MS: 336.2 (M+NH₄)⁺

### EXAMPLE A26

### 4-Hydroxy-5-isobutyl-3-[3-(4-methoxy-phenyl)-propionyl]-5H-furan-2-one

The title compound was obtained in comparable yields according to the procedures described for example A1 using 3-(4-methoxy-phenyl)-propionic acid (commercially available) instead of 3-methyl-butyric acid in step c).

MS: 336.2 (M+NH₄)⁺

### EXAMPLE A27

### 3-[3-(2,5-Dimethoxy-phenyl)-propionyl]-4-hydroxy-5-isobutyl-5H-furan-2-one

The title compound was obtained in comparable yields according to the procedures described for example A1 using 3-(2,5-dimethoxy-phenyl)-propionic acid (commercially available) instead of 3-methyl-butyric acid in step c).

MS: 349.4 (M+H)⁺

### EXAMPLE A28

### 3-[3-(4-Chloro-phenyl)-2-methyl-propionyl]-4-hydroxy-5-isobutyl-5H-furan-2-one

The title compound was obtained in comparable yields according to the procedures described for example A1 using 3-(4-chloro-phenyl)-2-methyl-propionic acid (prepared according to Ferorelli, S.; Loiodice, F.; Tortorella, V.; Amoroso, R.; Bettoni, G.; Conte-Camerino, D.; De Luca, A.; Farmaco (1997), 52(6-7), 367-374.) instead of 3-methyl-butyric acid in step c).

MS: 354.3 (M+NH₄)⁺

### EXAMPLE A29

### 3-[3-(4-tert-Butyl-phenyl)-2-methyl-propionyl]-4-hydroxy-5-isobutyl-5H-furan-2-one

The title compound was obtained in comparable yields according to the procedures described for example A1 using 3-(4-tert-Butyl-phenyl)-2-methyl-propionic acid (prepared according to Kuchar, Miroslav; Rejholec, Vaclav; Roubal, Zdenek; Nemecek, Oldrich; Collect. Czech. Chem. Commun. (1979), 44(1), 183-93) instead of 3-methyl-butyric acid in step c).

MS: 376.5 (M+NH₄)⁺

### EXAMPLE A30

### 4-Hydroxy-5-isobutyl-3-(3-phenyl-butyryl)-5H-furan-2-one

The title compound was obtained in comparable yields according to the procedures described for example A1 using 3-phenyl-butyric acid (commercially available) instead of 3-methyl-butyric acid in step c).

MS: 320.4 (M+NH₄)⁺

### EXAMPLE A31

### 4-Hydroxy-5-isobutyl-3-((R)—(R)-2-phenyl-cyclopropanecarbonyl)-5H-furan-2-one

The title compound was obtained in comparable yields according to the procedures described for example A1 using (R)—(R)-2-phenyl-cyclopropanecarboxylic acid (commercially available) instead of 3-methyl-butyric acid in step c).

MS: 318.3 (M+NH₄)⁺

### EXAMPLE A32

### 4-Hydroxy-5-isobutyl-3-[2-(2-methoxy-phenoxy)-acetyl]-5H-furan-2-one

The title compound was obtained in comparable yields according to the procedures described for example A1 using 2-(2-methoxy-phenoxy)-acetic acid (commercially available) instead of 3-methyl-butyric acid in step c).

MS: 319.1 (M−H)⁻

### EXAMPLE A33

### 4-Hydroxy-5-isobutyl-3-[2-(naphthalen-1-yloxy)-acetyl]-5H-furan-2-one

The title compound was obtained in comparable yields according to the procedures described for example A1 using 2-(naphthalen-1-yloxy)-acetic acid (commercially available) instead of 3-methyl-butyric acid in step c). MS: 339.0 (M−H)⁻

### EXAMPLE A34

### 4-Hydroxy-5-isobutyl-3-(2-phenoxy-propionyl)-5H-furan-2-one

The title compound was obtained in comparable yields according to the procedures described for example A1 using 2-phenoxy-propionic acid (commercially available) instead of 3-methyl-butyric acid in step c).

MS: 322.4 (M+NH₄)⁺

### EXAMPLE A35

### 4-Hydroxy-5-isobutyl-3-(4-phenyl-butyryl)-5H-furan-2-one

The title compound was obtained in comparable yields according to the procedures described for example A1 using 4-phenyl-butyric acid (commercially available) instead of 3-methyl-butyric acid in step c).

MS: 301.2 (M−H)⁻

### EXAMPLE A36

### 3-[4-(3,4-Dimethoxy-phenyl)-butyryl]-4-hydroxy-5-isobutyl-5H-furan-2-one

The title compound was obtained in comparable yields according to the procedures described for example A1 using 4-(3,4-dimethoxy-phenyl)-butyric acid (commercially available) instead of 3-methyl-butyric acid in step c).

MS: 380.3 (M+NH₄)⁺

### EXAMPLE A37

### 4-Hydroxy-5-isobutyl-3-((Z)-2-methyl-5-pyridin-3-yl-pent-4-enoyl)-5H-furan-2-one

The title compound was obtained in comparable yields according to the procedures described for example A1 using (Z)-2-methyl-5-pyridin-3-yl-pent-4-enoic acid (prepared according to Ziegler, Frederick E.; Sobolov, Susan B. Journal of the American Chemical Society (1990), 112(7), 2749-58) instead of 3-methyl-butyric acid in step c).

MS: 328.1 (M−H)⁻

### EXAMPLE A38

### 4-Hydroxy-5-isobutyl-3-((Z)-2-methyl-5-phenyl-hex-4-enoyl)-5H-furan-2-one

The title compound was obtained in comparable yields according to the procedures described for example A1 using (Z)-2-methyl-5-phenyl-hex-4-enoic acid (prepared according to Ziegler, Frederick E.; Sobolov, Susan B. Journal of the American Chemical Society (1990), 112(7), 2749-58) instead of 3-methyl-butyric acid in step c).

MS: 341.1 (M−H)⁻

### EXAMPLE A39

### 4-Hydroxy-3-(2-1H-indol-3-yl-acetyl)-5-isobutyl-5H-furan-2-one

The title compound was obtained in comparable yields according to the procedures described for example A1 using 2-1H-indol-3-yl-acetic acid (commercially available) instead of 3-methyl-butyric acid in step c).

MS: 314.2 (M+H)⁺

### EXAMPLE A40

### 4-Hydroxy-3-(3-1H-indol-3-yl-propionyl)-5-isobutyl-5H-furan-2-one

The title was obtained in comparable yields according to the procedures described for example A1 using 3-1H-indol-3-yl-propionic acid (commercially available) instead of 3-methyl-butyric acid in step c). MS: 345.3 (M+NH₄)⁺

### EXAMPLE A41

### 4-Hydroxy-5-isobutyl-3-(2-naphthalen-2-yl-acetyl)-5H-furan-2-one

The title compound was obtained in comparable yields according to the procedures described for example A1 using 2-naphthalen-2-yl-acetic acid (commercially available) instead of 3-methyl-butyric acid in step c).

MS: 342.2 (M+NH₄)⁺

### EXAMPLE A42

### 3-[2-(2-Acetyl-1,2-dihydro-isoquinolin-1-yl)-acetyl]-4-hydroxy-5-isobutyl-5H-furan-2-one

The title compound was obtained in comparable yields according to the procedures described for example A1 using 2-(2-Acetyl-1,2-dihydro-isoquinolin-1-yl)-acetic acid (commercially available) instead of 3-methyl-butyric acid in step c).

MS: 368.0 (M−H)⁻

### EXAMPLE A43

### 3-Diphenylacetyl-4-hydroxy-5-isobutyl-5H-furan-2-one

The title compound was obtained in comparable yields according to the procedures described for example A1 using diphenylacetic acid (commercially available) instead of 3-methyl-butyric acid in step c).

MS: 368.3 (M+NH₄)⁺

### EXAMPLE A44

### 3-(3,3-Diphenyl-propionyl)-4-hydroxy-5-isobutyl-5H-furan-2-one

The title compound was obtained in comparable yields according to the procedures described for example A1 using 3,3-Diphenyl-propionic acid (commercially available) instead of 3-methyl-butyric acid in step c).

MS: 363.1 (M−H)⁻

### EXAMPLE A45

### 4-Hydroxy-5-isobutyl-3-[(9H-thioxanthen-9-yl)-acetyl]-5H-furan-2-one

The title compound was obtained in comparable yields according to the procedures described for example A1 using (9H-thioxanthen-9-yl)-acetic acid (prepared according to Jilek, Jiri O.; Holubek, Jiri; Svatek, Emil; Ryska, Miroslav; Pomykacek, Josef; Protiva, Miroslav. Collection of Czechoslovak Chemical Communications (1979), 44(7), 2124-38) instead of 3-methyl-butyric acid in step c).

MS: 312.4 (M+NH₄)⁺

### EXAMPLE A46

### 3-[(10,11-Dihydro-5H-dibenzo[a,d]cyclohepten-5-yl)-acetyl]-4-hydroxy-5-isobutyl-5H-furan-2-one

The title compound was obtained in comparable yields according to the procedures described for example A1 using (10,11-dihydro-5H-dibenzo[a,d]cyclohepten-5-yl)-acetic acid (prepared according to Tucker, Thomas J.; Lumma, William C.; Lewis, S. Dale; Gardell, Stephen J.; Lucas, Bobby J.; Sisko, Jack T.; Lynch, Joseph J.; Lyle, Elizabeth A.; Baskin, Elizabeth P.; Woltmann, Richard F.; Appleby, Sandra D.; Chen, I-Wu; Dancheck, Kimberley B.; Naylor-Olsen, Adel M.; Krueger, Julie A.; Cooper, Carolyn M.; Vacca, Joseph P. Journal of Medicinal Chemistry (1997), 40(22), 3687-3693) instead of 3-methyl-butyric acid in step c).

MS: 308.4 (M+NH₄)⁺

### EXAMPLE B1

### 4-Hydroxy-3-(3-methylsulfanyl-propionyl)-5-(2-methylsulfanyl-propyl)-5H-furan-2-one

### a) 4-Methoxy-5-(2-methyl-sulfanyl-propyl)-5H-furan-2-one

To a solution of 20 ml of LDA (2M in THF) and 130 ml of THF was added at −95° C. to −100° C. a solution of 5.47 g of 3(E)-methoxy-acrylic acid methyl ester in 4.5 ml of THF within 1 min, stirring was continued at the same temperature for 5 min, which was followed by the addition of a pre-cooled (−78° C.) solution of 33 mmole of the 3-methylsulfanyl-butyraldehyde in 4.5 ml of THF within 2 min and stirring was continued at −100° C. for 30 min and at −78° C. for 1 h. The cold solution was poured onto 130 ml of ice-water, the pH was adjusted to 4 with 6.5 ml of aqueous HCl (37%) and the layers were separated. The aqueous layer was extracted twice with dichloromethane, the organic layers were washed with brine, dried and evaporated. The residue was chromatographed on silica (n-heptane/AcOEt, various ratios) to give the 4-methoxy-5-(2-methyl-sulfanyl-propyl)-5H-furan-2-one in 30-40% yield.

MS: 202.3 (M)⁺

### b) 4-Hydroxy-5-(2-methylsulfanyl-propyl)-5H-furan-2-one

A mixture of the 4-methoxy-5-(2-methyl-sulfanyl-propyl)-5H-furan-2-one (10 mmole) and 15 ml of aqueous HCl (37%) was stirred at 40° C. until completion of the reaction. The suspension was filtered and the residue washed with ice-cold water and dried. An oily reaction mixture was extracted with dichloromethane, the organic layers were washed with brine, dried and evaporated. The residue was either triturated with AcOEt/hexane or chromatographed with dichloromethane/MeOH (various ratios) to give the 4-hydroxy-5-(2-methylsulfanyl-propyl)-5H-furan-2-one in 60-90% yield.

MS: 188.0 (M)⁺

### c) 4-Hydroxy-3-(3-methylsulfanyl-propionyl)-5-(2-methylsulfanyl-propyl)-5H-furan-2-one

To as suspension of the 4-hydroxy-5-(2-methylsulfanyl-propyl)-5H-furan-2-one (0.2 mmole), NEt₃ (0.68 mmole), DMAP (0.066 mmole) and EDC (0.44 mmole) in 2 ml of THF was added at 22° C. 3-methylsulfanyl-propionic acid (0.22 mmole) (commercially available) and stirring was continued until completion of the reaction. The pH of the reaction mixture was adjusted to 3 using aqueous HCl (2 N), the aqueous solution was saturated with NaCl, the organic layer was separated, washed with brine dried and evaporated. The residue was purified on preparative HPLC (RP-18, CH₃CN/H₂O, gradient) to give the 4-hydroxy-3-(3-methylsulfanyl-propionyl)-5-(2-methylsulfanyl-propyl)-5H-furan-2-one in 10-60% yield.

MS: 289.0 (M−H)⁻

### EXAMPLE B2

### 3-Cyclopropanecarbonyl-4-hydroxy-5-(2-methylsulfanyl-propyl)-5H-furan-2-one

The title compound was obtained in comparable yields according to the procedures described for example B1 using cyclopropanecarboxylic acid (commercially available) instead of 3-methylsulfanyl-propionic acid in step c).

MS: 255.0 (M−H)⁻

### EXAMPLE B3

### 4-Hydroxy-5-(2-methylsulfanyl-propyl)-3-(2,2,3,3-tetramethyl-cyclopropanecarbonyl)-5H-furan-2-one

The title compound was obtained in comparable yields according to the procedures described for example B1 using 2,2,3,3-tetramethyl-cyclopropanecarboxylic acid (commercially available) instead of 3-methylsulfanyl-propionic acid in step c).

MS: 311.0 (M−H)⁻

### EXAMPLE B4

### 4-Hydroxy-5-(2-methylsulfanyl-propyl)-3-(tetrahydro-furan-2-carbonyl)-5H-furan-2-one

The title compound was obtained in comparable yields according to the procedures described for example B1 using tetrahydro-furan-2-carboxylic acid (commercially available) instead of 3-methylsulfanyl-propionic acid in step c).

MS: 285.0 (M−H)⁻

### EXAMPLE B5

### 3-Cyclohexanecarbonyl-4-hydroxy-5-(2-methylsulfanyl-propyl)-5H-furan-2-one

The title compound was obtained in comparable yields according to the procedures described for example B1 using cyclohexanecarboxylic acid (commercially available) instead of 3-methylsulfanyl-propionic acid in step c).

MS: 297.2 (M−H)⁻

### EXAMPLE B6

### 3-(4-tert-Butyl-cyclohexanecarbonyl)-4-hydroxy-5-(2-methylsulfanyl-propyl)-5H-furan-2-one

The title compound was obtained in comparable yields according to the procedures described for example B1 using 4-tert-butyl-cyclohexanecarboxylic acid (commercially available) instead of 3-methylsulfanyl-propionic acid in step c).

MS: 353.2 (M−H)⁻

### EXAMPLE B7

### 3-(2-Cyclohexyl-acetyl)-4-hydroxy-5-(2-methylsulfanyl-propyl)-5H-furan-2-one

The title compound was obtained in comparable yields according to the procedures described for example B1 using 2-cyclohexyl-acetic acid (commercially available) instead of 3-methylsulfanyl-propionic acid in step c).

MS: 311.0 (M−H)⁻

### EXAMPLE B8

### 3-(4-Cyclohexyl-butyryl)-4-hydroxy-5-(2-methylsulfanyl-propyl)-5H-furan-2-one

The title compound was obtained in comparable yields according to the procedures described for example B1 using 4-Cyclohexyl-butyric acid (commercially available) instead of 3-methylsulfanyl-propionic acid in step c).

MS: 339.1 (M−H)⁻

### EXAMPLE B9

### 4-Hydroxy-5-(2-methylsulfanyl-propyl)-3-phenylacetyl-5H-furan-2-one

The title compound was obtained in comparable yields according to the procedures described for example B1 using phenylacetic acid (commercially available) instead of 3-methylsulfanyl-propionic acid in step c).

MS: 305.0 (M−H)⁻

### EXAMPLE B10

### 4-Hydroxy-3-[2-(4-methoxy-3-methyl-phenyl)-acetyl]-5-(2-methylsulfanyl-propyl)-5H-furan-2-one

The title compound was obtained in comparable yields according to the procedures described for example B1 using 2-(4-methoxy-3-methyl-phenyl)-acetic acid (commercially available) instead of 3-methylsulfanyl-propionic acid in step c).

MS: 349.2 (M−H)⁻

### EXAMPLE B11

### 3-[2-(3,5-Dimethoxy-phenyl)-acetyl]-4-hydroxy-5-(2-methylsulfanyl-propyl)-5H-furan-2-one

The title compound was obtained in comparable yields according to the procedures described for example B1 using 2-(3,5-dimethoxy-phenyl)-acetic acid (commercially available) instead of 3-methylsulfanyl-propionic acid in step c).

MS: 365.1 (M−H)⁻

### EXAMPLE B12

### 3-[2-(2,4-Dimethoxy-phenyl)-acetyl]-4-hydroxy-5-(2-methylsulfanyl-propyl)-5H-furan-2-one

The title compound was obtained in comparable yields according to the procedures described for example B1 using 2-(2,4-dimethoxy-phenyl)-acetic acid (commercially available) instead of 3-methylsulfanyl-propionic acid in step c).

MS: 365.1 (M−H)⁻

### EXAMPLE B13

### 3-[2-(2,5-Dimethoxy-phenyl)-acetyl]-4-hydroxy-5-(2-methylsulfanyl-propyl)-5H-furan-2-one

The title compound was obtained in comparable yields according to the procedures described for example B1 using 2,5-Dimethoxy-phenyl)-acetic acid (commercially available) instead of 3-methylsulfanyl-propionic acid in step c).

MS: 365.1 (M−H)⁻

### EXAMPLE B14

### 4-Hydroxy-5-(2-methylsulfanyl-propyl)-3-(2-naphthalen-2-yl-acetyl)-5H-furan-2-one

The title compound was obtained in comparable yields according to the procedures described for example B1 using 2,2,3,3-tetramethyl-cyclopropanecarboxylic acid (commercially available) instead of 3-methylsulfanyl-propionic acid in step c).

MS: 355.1 (M−H)⁻

### EXAMPLE B15

### 4-Hydroxy-5-(2-methylsulfanyl-propyl)-3-(2-phenyl-propionyl)-5H-furan-2-one

The title compound was obtained in comparable yields according to the procedures described for example B1 using 2-phenyl-propionic acid (commercially available) instead of 3-methylsulfanyl-propionic acid in step c).

MS: 319.1 (M−H)⁻

### EXAMPLE B16

### 4-Hydroxy-5-(2-methylsulfanyl-propyl)-3-(2-phenyl-butyryl)-5H-furan-2-one

The title compound was obtained in comparable yields according to the procedures described for example B1 using 2-phenyl-butyric acid (commercially available) instead of 3-methylsulfanyl-propionic acid in step c).

MS: 333.0 (M−H)⁻

### EXAMPLE B17

### 4-Hydroxy-3-[2-(6-methoxy-naphthalen-2-yl)-propionyl]-5-(2-methylsulfanyl-propyl)-5H-furan-2-one

The title compound was obtained in comparable yields according to the procedures described for example B1 using 2-(6-methoxy-naphthalen-2-yl)-propionic acid (commercially available) instead of 3-methylsulfanyl-propionic acid in step c).

MS: 399.2 (M−H)⁻

### EXAMPLE B18

### 4-Hydroxy-5-(2-methylsulfanyl-propyl)-3-(3-phenyl-propionyl)-5H-furan-2-one

The title compound was obtained in comparable yields according to the procedures described for example B1 using 3-phenyl-propionic acid (commercially available) instead of 3-methylsulfanyl-propionic acid in step c).

MS: 319.1 (M−H)⁻

### EXAMPLE B19

### 4-Hydroxy-5-(2-methylsulfanyl-propyl)-3-(3-m-tolyl-propionyl)-5H-furan-2-one

The title compound was obtained in comparable yields according to the procedures described for example B1 using 3-m-tolyl-propionic acid (commercially available) instead of 3-methylsulfanyl-propionic acid in step c).

MS: 333.1 (M−H)⁻

### EXAMPLE B20

### 4-Hydroxy-3-[3-(3-methoxy-phenyl)-propionyl]-5-(2-methylsulfanyl-propyl)-5H-furan-2-one

The title compound was obtained in comparable yields according to the procedures described for example B1 using 3-(3-methoxy-phenyl)-propionic acid (commercially available) instead of 3-methylsulfanyl-propionic acid in step c).

MS: 349.2 (M−H)⁻

### EXAMPLE B21

### 4-Hydroxy-3-[3-(4-methoxy-phenyl)-propionyl]-5-(2-methylsulfanyl-propyl)-5H-furan-2-one

The title compound was obtained in comparable yields according to the procedures described for example B1 using 3-(4-methoxy-phenyl)-propionic acid (commercially available) instead of 3-methylsulfanyl-propionic acid in step c).

MS: 349.2 (M−H)⁻

### EXAMPLE B22

### 3-[3-(2,5-Dimethoxy-phenyl)-propionyl]-4-hydroxy-5-(2-methylsulfanyl-propyl)-5H-furan-2-one

The title compound was obtained in comparable yields according to the procedures described for example B1 using 2,5-dimethoxy-phenic acid (commercially available) instead of 3-methylsulfanyl-propionic acid in step c).

MS: 379.1 (M−H)⁻

### EXAMPLE B23

### 3-[3-(4-tert-Butyl-phenyl)-2-methyl-propionyl]-4-hydroxy-5-(2-methylsulfanyl-propyl)-5H-furan-2-one

The title compound was obtained in comparable yields according to the procedures described for example B1 using 3-(4-tert-Butyl-phenyl)-2-methyl-propionic acid (prepared according to Kuchar, Miroslav; Rejholec, Vaclav; Roubal, Zdenek; Nemecek, Oldrich; Collect. Czech. Chem. Commun. (1979), 44(1), 183-93) instead of 3-methylsulfanyl-propionic acid in step c).

MS: 389.2 (M−H)⁻

### EXAMPLE B24

### 4-Hydroxy-5-(2-methylsulfanyl-propyl)-3-(3-phenyl-butyryl)-5H-furan-2-one

The title compound was obtained in comparable yields according to the procedures described for example B1 using 3-phenyl-butyric acid (commercially available) instead of 3-methylsulfanyl-propionic acid in step c).

MS: 333.0 (M−H).

### EXAMPLE B25

### 4-Hydroxy-5-(2-methylsulfanyl-propyl)-3-((R)—(R)-2-phenyl-cyclopropanecarbonyl)-5H-furan-2-one

The title compound was obtained in comparable yields according to the procedures described for example B1 using 2-((R)—(R)-2-phenyl-cyclopropanecarboxylic acid (commercially available) instead of 3-methylsulfanyl-propionic acid in step c).

MS: 331.0 (M−H)⁻

### EXAMPLE B26

### 4-Hydroxy-3-[2-(2-methoxy-phenoxy)-acetyl]-5-(2-methylsulfanyl-propyl)-5H-furan-2-one

The title compound was obtained in comparable yields according to the procedures described for example B1 using 2-(2-methoxy-phenoxy)-acetic acid (commercially available) instead of 3-methylsulfanyl-propionic acid in step c).

MS: 351.1 (M−H)⁻

### EXAMPLE B27

### 3-[2-(2,3-Dimethyl-phenoxy)-acetyl]-4-hydroxy-5-(2-methylsulfanyl-propyl)-5H-furan-2-one

The title compound was obtained in comparable yields according to the procedures described for example B1 using 2-(2,3-dimethyl-phenoxy)-acetic acid (commercially available) instead of 3-methylsulfanyl-propionic acid in step c).

MS: 349.2 (M−H)⁻

### EXAMPLE B28

### 4-Hydroxy-5-(2-methylsulfanyl-propyl)-3-(2-phenoxy-propionyl)-5H-furan-2-one

The title compound was obtained in comparable yields according to the procedures described for example B1 using 2-phenoxy-propionic acid (commercially available) instead of 3-methylsulfanyl-propionic acid in step c).

MS: 335.0 (M−H)⁻

### EXAMPLE B29

### 4-Hydroxy-5-(2-methylsulfanyl-propyl)-3-(2-phenoxy-butyryl)-5H-furan-2-one

The title compound was obtained in comparable yields according to the procedures described for example B1 using 2-phenoxy-butyric acid (commercially available) instead of 3-methylsulfanyl-propionic acid in step c).

MS: 349.2 (M−H)⁻

### EXAMPLE B30

### 4-Hydroxy-5-(2-methylsulfanyl-propyl)-3-[2-(naphthalen-1-yloxy)-acetyl]-5H-furan-2-one

The title compound was obtained in comparable yields according to the procedures described for example B1 using 2-(naphthalen-1-yloxy)-acetic acid (commercially available) instead of 3-methylsulfanyl-propionic acid in step c).

MS: 371.1 (M−H)⁻

### EXAMPLE B31

### 4-Hydroxy-5-(2-methylsulfanyl-propyl)-3-(4-phenyl-butyryl)-5H-furan-2-one

The title compound was obtained in comparable yields according to the procedures described for example B1 using 4-phenyl-butyric acid (commercially available) instead of 3-methylsulfanyl-propionic acid in step c).

MS: 333.1 (M−H)⁻

### EXAMPLE B32

### 3-[4-(3,4-Dimethoxy-phenyl)-butyryl]-4-hydroxy-5-(2-methylsulfanyl-propyl)-5H-furan-2-one

The title compound was obtained in comparable yields according to the procedures described for example B1 using 4-(3,4-dimethoxy-phenyl)-butyric acid (commercially available) instead of 3-methylsulfanyl-propionic acid in step c).

MS: 393.0 (M−H)⁻

### EXAMPLE B33

### 4-Hydroxy-3-[(H-indol-3-yl)-acetyl]-5-(2-methylsulfanyl-propyl)-5H-furan-2-one

The title compound was obtained in comparable yields according to the procedures described for example B1 using (1H-indol-3-yl)-acetic acid (commercially available) instead of 3-methylsulfanyl-propionic acid in step c).

MS: 344.0 (M−H)⁻

### EXAMPLE B34

### 4-Hydroxy-3-(3-1H-indol-3-yl-propionyl)-5-(2-methylsulfanyl-propyl)-5H-furan-2-one

The title compound was obtained in comparable yields according to the procedures described for example B1 using 3-1H-indol-3-yl-propionic acid (commercially available) instead of 3-methylsulfanyl-propionic acid in step c).

MS: 358.0 (M−H)⁻

### EXAMPLE B35

### 3-[2-(2-Acetyl-1,2-dihydro-isoquinolin-1-yl)-acetyl]-4-hydroxy-5-(2-methylsulfanyl-propyl)-5H-furan-2-one

The title compound was obtained in comparable yields according to the procedures described for example B1 using 2-(2-acetyl-1,2-dihydro-isoquinolin-1-yl)-acetic acid (commercially available) instead of 3-methylsulfanyl-propionic acid in step c).

MS: 400.2 (M−H)⁻

### EXAMPLE B36

### 3-Diphenylacetyl-4-hydroxy-5-(2-methylsulfanyl-propyl)-5H-furan-2-one

The title compound was obtained in comparable yields according to the procedures described for example B1 using diphenylacetic acid (commercially available) instead of 3-methylsulfanyl-propionic acid in step c).

MS: 341.1 (M−H)⁻

### EXAMPLE B37

### 3-(3,3-Diphenyl-propionyl)-4-hydroxy-5-(2-methylsulfanyl-propyl)-5H-furan-2-one

The title compound was obtained in comparable yields according to the procedures described for example B1 using 3,3-diphenyl-propionic acid (commercially available) instead of 3-methylsulfanyl-propionic acid in step c).

MS: 394.9 (M−H)⁻

### EXAMPLE B38

### 4-Hydroxy-5-(2-methylsulfanyl-propyl)-3-(2-9H-thioxanthen-9-yl-acetyl)-5H-furan-2-one

The title compound was obtained in comparable yields according to the procedures described for example B1 using 2-9H-thioxanthen-9-yl-acetic acid (prepared according to Jilek, Jiri O.; Holubek, Jiri; Svatek, Emil; Ryska, Miroslav; Pomykacek, Josef; Protiva, Miroslav. Collection of Czechoslovak Chemical Communications (1979), 44(7), 2124-2138) instead of 3-methylsulfanyl-propionic acid in step c).

MS: 425.2 (M−H)⁻

### EXAMPLE B39

### 3-(2-10,11-Dihydro-5H-dibenzo[a,d]cyclohepten-5-yl-acetyl)-4-hydroxy-5-(2-methylsulfanyl-propyl)-5H-furan-2-one

The title compound was obtained in comparable yields according to the procedures described for example B1 using 2-10,11-Dihydro-5H-dibenzo[a,d]cyclohepten-5-yl-acetic acid (prepared according to Tucker, Thomas J.; Lumma, William C.; Lewis, S. Dale; Gardell, Stephen J.; Lucas, Bobby J.; Sisko, Jack T.; Lynch, Joseph J.; Lyle, Elizabeth A.; Baskin, Elizabeth P.; Woltmann, Richard F.; Appleby, Sandra D.; Chen, I-Wu; Dancheck, Kimberley B.; Naylor-Olsen, Adel M.; Krueger, Julie A.; Cooper, Carolyn M.; Vacca, Joseph P. Journal of Medicinal Chemistry (1997), 40(22), 3687-3693) instead of 3-methylsulfanyl-propionic acid in step c).

MS: 421.2 (M−H)⁻

### EXAMPLE C1

### 3-Cyclohexanecarbonyl-5-cyclohexylmethyl-4-hydroxy-5H-furan-2-one

### a) 5-Cyclohexylmethyl-4-methoxy-5H-furan-2-one

To a solution of 20 ml of LDA (2M in THF) and 130 ml of THF was added at −95° C. to −100° C. a solution of 5.47 g of 3(E)-methoxy-acrylic acid methyl ester in 4.5 ml of THF within 1 min, stirring was continued at the same temperature for 5 min, which was followed by the addition of a pre-cooled (−78° C.) solution of 33 mmole of the cyclohexyl-acetaldehyde in 4.5 ml of THF within 2 min and stirring was continued at −100° C. for 30 min and at −78° C. for 1 h. The cold solution was poured onto 130 ml of ice-water, the pH was adjusted to 4 with 6.5 ml of aqueous HCl (37%) and the layers were separated. The aqueous layer was extracted twice with dichloromethane, the organic layers were washed with brine, dried and evaporated. The residue was chromatographed on silica (n-heptane/AcOEt, various ratios) to give the 5-cyclohexylmethyl-4-methoxy-5H-furan-2-one in 30-40% yield.

MS: 114.0 (M-C₇H₁₂)⁺

### b) 5-Cyclohexylmethyl-4-hydroxy-5H-furan-2-one

A mixture of the 5-cyclohexylmethyl-4-methoxy-5H-furan-2-one (10 mmole) and 15 ml of aqueous HCl (37%) was stirred at 40° C. until completion of the reaction. The suspension was filtered and the residue washed with ice-cold water and dried. An oily reaction mixture was extracted with dichloromethane, the organic layers were washed with brine, dried and evaporated. The residue was either triturated with AcOEt/hexane or chromatographed with dichloromethane/MeOH (various ratios) to give 5-cyclohexylmethyl-4-hydroxy-5H-furan-2-one in 60-90% yield.

MS: 197.2 (M+H)⁺

### c) 3-Cyclohexanecarbonyl-5-cyclohexylmethyl-4-hydroxy-5H-furan-2-one

To as suspension of the 5-cyclohexylmethyl-4-hydroxy-5H-furan-2-one (0.2 mmole), NEt₃ (0.68 mmole), DMAP (0.066 mmole) and EDC (0.44 mmole) in 2 ml of THF was added at 22° C. cyclohexanecarboxylic acid (0.22 mmole) (commercially available) and stirring was continued until completion of the reaction. The pH of the reaction mixture was adjusted to 3 using aqueous HCl (2 N), the aqueous solution was saturated with NaCl, the organic layer was separated, washed with brine dried and evaporated. The residue was purified on preparative HPLC (RP-18, CH₃CN/H₂O, gradient) to give the 3-cyclohexanecarbonyl-5-cyclohexylmethyl-4-hydroxy-5H-furan-2-one in 10-60% yield.

MS: 305.1 (M−H)⁻

### EXAMPLE C2

### 3-Cyclohexylacetyl-5-cyclohexylmethyl-4-hydroxy-5H-furan-2-one

The title compound was obtained in comparable yields according to the procedures described for example C1 using cyclohexylacetic acid (commercially available) instead of cyclohexanecarboxylic acid in step c).

MS: 319.2 (M−H)⁻

### EXAMPLE C3

### 5-Cyclohexylmethyl-3-(3-cyclohexyl-propionyl)-4-hydroxy-5H-furan-2-one

The title compound was obtained in comparable yields according to the procedures described for example C1 using 3-cyclohexyl-propionic acid (commercially available) instead of cyclohexanecarboxylic acid in step c).

MS: 333.3 (M−H)⁻

### EXAMPLE C4

### 3-(4-Cyclohexyl-butyryl)-5-cyclohexylmethyl-4-hydroxy-5H-furan-2-one

The title compound was obtained in comparable yields according to the procedures described for example C1 using 4-cyclohexyl-butyric acid (commercially available) instead of cyclohexanecarboxylic acid in step c).

MS: 347.3 (M−H)⁻

### EXAMPLE C5

### 4-Chloro-N-[3-cyclohexyl-1-(5-cyclohexylmethyl-4-hydroxy-2-oxo-2,5-dihydro-furan-3-carbonyl)-propyl]-benzenesulfonamide

The title compound was obtained in comparable yields according to the procedures described for example C1 using

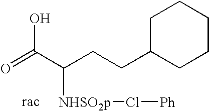

(Prepared from the commercially available amine and the corresponding sulfochloride) instead of cyclohexanecarboxylic acid in step c).

MS: 536.3 (M−H)⁻

### EXAMPLE C6

### 5-Cyclohexylmethyl-3-(5-cyclohexyl-pentanoyl)-4-hydroxy-5H-furan-2-one

The title compound was obtained in comparable yields according to the procedures described for example C1 using 5-cyclohexyl-pentanoic acid (commercially available) instead of cyclohexanecarboxylic acid in step c).

MS: 361.3 (M−H)⁻

### EXAMPLE C7

### 5-Cyclohexylmethyl-4-hydroxy-3-(2-methyl-3-phenyl-propionyl)-5H-furan-2-one

The title compound was obtained in comparable yields according to the procedures described for example C1 using 2-methyl-3-phenyl-propionic acid (commercially available) instead of cyclohexanecarboxylic acid in step c).

MS: 341.1 (M−H)⁻

### EXAMPLE C8

### 3-[3-(4-tert-Butyl-phenyl)-2-methyl-propionyl]-5-cyclohexylmethyl-4-hydroxy-5H-furan-2-one

The title compound was obtained in comparable yields according to the procedures described for example C1 using (4-tert-butyl-phenyl)-2-methyl-propionic acid (prepared according to Kuchar, Miroslav; Rejholec, Vaclav; Roubal, Zdenek; Nemecek, Oldrich; Collect. Czech. Chem. Commun. (1979), 44(1), 183-93) instead of cyclohexanecarboxylic acid in step c).

MS: 397.2 (M−H)⁻

### EXAMPLE C9

### 3-[3-(4-Benzyloxy-phenyl)-2-methyl-propionyl]-5-cyclohexylmethyl-4-hydroxy-5H-furan-2-one

The title compound was obtained in comparable yields according to the procedures described for example C1 using 3-(4-benzyloxy-phenyl)-2-methyl-propionic acid (prepared according to Hitchcock, Janice M.; Sorenson, Stephen M.; Dudley, Mark W.; Peet, Norton P; WO 9419349 A1 (1994)) instead of cyclohexanecarboxylic acid in step c).

MS: 447.2 (M−H)⁻

### EXAMPLE C10

(2-{4-[3-(5-Cyclohexylmethyl-4-hydroxy-2-oxo-2,5-dihydro-furan-3-yl)-2-methyl-3-oxo-propyl]-phenylcarbamoyl}-ethyl)-carbamic acid tert-butyl ester

The title compound was prepared from the corresponding BOC-protected precursor by deprotection using CF₃COOH and was obtained in comparable yields according to the procedures described for example C1 using

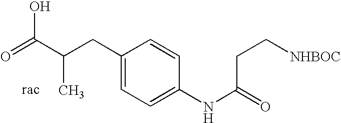

(prepared from the aniline (Biagi, Giuliana; Dell'omodarme, Giuliana; Giorgi, Irene; Livi, Oreste; Scartoni, Valerio; Farmaco (1992), 47(1), 91-8) and the corresponding acid) instead of cyclohexanecarboxylic acid in step c).

MS: 527.3 (M−H)⁻

### EXAMPLE C11

### N-(2-{4-[3-(5-Cyclohexylmethyl-4-hydroxy-2-oxo-2,5-dihydro-furan-3-yl)-2-methyl-3-oxo-propyl]-phenyl}-ethyl)-benzenesulfonamide

The title compound was obtained in comparable yields according to the procedures described for example C1 using

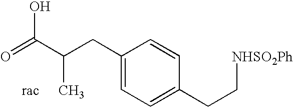

(prepared from the amine (Bosies, Elmar; Heerdt, Ruth; Kuhnle, Hans Frieder; Schmidt, Felix H.; Stach, Kurt; U.S. Pat. No. 4,113,871 (1980), 13 pp) and the corresponding sulfochloride)) instead of cyclohexanecarboxylic acid in step c).

MS: 524.2 (M−H)⁻

### EXAMPLE C12

### 5-Chloro-N-(2-{4-[3-(5-cyclohexylmethyl-4-hydroxy-2-oxo-2,5-dihydro-furan-3-yl)-2-methyl-3-oxo-propyl]-phenyl}-ethyl)-2-methoxy-benzamide

The title compound was prepared from the corresponding BOC-protected precursor by deprotection using CF₃COOH and was obtained in comparable yields according to the procedures described for example C1 using

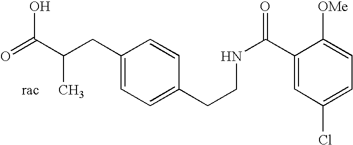

(prepared according to Bosies, Elmar; Heerdt, Ruth; Kuhnle, Hans Frieder; Schmidt, Felix H.; Stach, Kurt; U.S. Pat. No. 4,113,871 (1980), 13 pp.) instead of cyclohexanecarboxylic acid in step c).

MS: 552.1 (M−H)⁻

### EXAMPLE C13

### [1-(4-Benzyloxy-benzyl)-2-(5-cyclohexylmethyl-4-hydroxy-2-oxo-2,5-dihydro-furan-3-yl)-2-oxo-ethyl]-carbamic acid tert-butyl ester

The title compound was obtained in comparable yields according to the procedures described for example C1 using

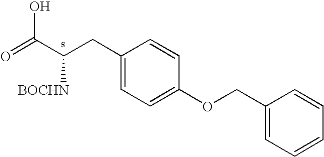

(commercially available) instead of cyclohexanecarboxylic acid in step c).

MS: 567.6 (M+NH₄)⁺

### EXAMPLE C14

### [2-(5-Cyclohexylmethyl-4-hydroxy-2-oxo-2,5-dihydro-furan-3-yl)-1-(4-hydroxy-benzyl)-2-oxo-ethyl]-carbamic acid tert-butyl ester

The title compound was obtained in comparable yields according to the procedures described for example C1 using

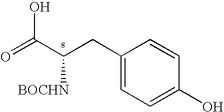

(commercially available) instead of cyclohexanecarboxylic acid in step c).

MS: 458.4 (M−H)⁻

### EXAMPLE C15

### 3-[2-Amino-3-(4-hydroxy-phenyl)-propionyl]-5-cyclohexylmethyl-4-hydroxy-5H-furan-2-one; compound with trifluoro-acetic acid

The title compound was prepared from the corresponding BOC-protected precursor (Example C14) by deprotection using CF₃COOH.

MS: 360.2 (M+H)⁺

### EXAMPLE C16

### 5-Cyclohexylmethyl-4-hydroxy-3-[(2-methoxy-phenoxy)-acetyl]-5H-furan-2-one

The title compound was obtained in comparable yields according to the procedures described for example C1 using (2-methoxy-phenoxy)-acetic acid (commercially available) instead of cyclohexanecarboxylic acid in step c).

MS: 359.0 (M−H)⁻

### EXAMPLE C17

### 5-Cyclohexylmethyl-4-hydroxy-3-[(H-indol-3-yl)-acetyl]-5H-furan-2-one

The title compound was obtained in comparable yields according to the procedures described for example C1 using (1H-indol-3-yl)-acetic acid (commercially available) instead of cyclohexanecarboxylic acid in step c).

MS: 352.2 (M−H)⁻

### EXAMPLE C18

### 5-Cyclohexylmethyl-4-hydroxy-3-[(1-methyl-1H-indol-3-yl)-acetyl]-5H-furan-2-one

The title compound was obtained in comparable yields according to the procedures described for example C1 using (1-methyl-1H-indol-3-yl)-acetic acid (commercially available) instead of cyclohexanecarboxylic acid in step c).

MS: 366.0 (M−H)⁻

### EXAMPLE C19

### 5-Cyclohexylmethyl-3-{[1-(4-fluoro-benzyl)-1H-indol-3-yl]-acetyl}-4-hydroxy-5H-furan-2-one

The title compound was obtained in comparable yields according to the procedures described for example C1 using 1-(4-fluoro-benzyl)-1H-indol-3-yl]-acetic acid (commercially available) instead of cyclohexanecarboxylic acid in step c).

MS: 462.3 (M−H)⁻

### EXAMPLE C20

### 3-{[1-(4-Chloro-benzyl)-5-methoxy-2-methyl-1H-indol-3-yl]-acetyl}-5-cyclohexylmethyl-4-hydroxy-5H-furan-2-one

The title compound was obtained in comparable yields according to the procedures described for example C1 using 1-(4-Chloro-benzyl)-5-methoxy-2-methyl-1H-indol-3-yl]-acetic acid (prepared by alkylation of the indole with the corresponding p-chlorophenyl methyl bromide) instead of cyclohexanecarboxylic acid in step c).

MS: 520.3 (M−H)⁻

### EXAMPLE C21

### 3-{[1-(4-Chloro-benzoyl)-5-methoxy-2-methyl-1H-indol-3-yl]-acetyl}-5-cyclohexylmethyl-4-hydroxy-5H-furan-2-one

The title compound was obtained in comparable yields according to the procedures described for example C1 using 1-(4-Chloro-benzoyl)-5-methoxy-2-methyl-1H-indol-3-yl]-acetic acid (prepared by acylation of the indole with the corresponding acid chloride) instead of cyclohexanecarboxylic acid in step c).

MS: 534.2 (M−H)⁻

### EXAMPLE C22

### 5-Cyclohexylmethyl-4-hydroxy-3-(indol-1-yl-acetyl)-5H-furan-2-one

The title compound was obtained in comparable yields according to the procedures described for example C1 using indol-1-yl-acetic acid (commercially available) instead of cyclohexanecarboxylic acid in step c).

MS: 352.2 (M−H)⁻

### EXAMPLE C23

### 5-Cyclohexylmethyl-4-hydroxy-3-(3-1H-indol-3-yl-propionyl)-5H-furan-2-one

The title compound was obtained in comparable yields according to the procedures described for example C1 using 3-1H-indol-3-yl-propionic acid (commercially available) instead of cyclohexanecarboxylic acid in step c).

MS: 366.1 (M−H)⁻

### EXAMPLE C24

### 5-Cyclohexylmethyl-4-hydroxy-3-[(2-methyl-benzofuran-3-yl)-acetyl]-5H-furan-2-one

The title compound was obtained in comparable yields according to the procedures described for example C1 using 2-methyl-benzofuran-3-yl)-acetic acid (prepared according to Wu, Jing et al.; WO 9828268 (1998), 889 pp.) instead of cyclohexanecarboxylic acid in step c).

MS: 367.2 (M−H)⁻

### EXAMPLE C25

### 3-[(5-Chloro-benzofuran-3-yl)-acetyl]-5-cyclohexylmethyl-4-hydroxy-5H-furan-2-one

The title compound was obtained in comparable yields according to the procedures described for example C1 using 5-Chloro-benzofuran-3-yl)-acetic acid (prepared according to Aeggi, Knut A.; Renner, Ulrich; CH504429 (1971), 7 pp.) instead of cyclohexanecarboxylic acid in step c).

MS: 387.2 (M−H)⁻

### EXAMPLE C26

### 3-(Benzo[b]thiophen-3-yl-acetyl)-5-cyclohexylmethyl-4-hydroxy-5H-furan-2-one

The title compound was obtained in comparable yields according to the procedures described for example C1 using Benzo[b]thiophen-3-yl-acetic acid (commercially available) instead of cyclohexanecarboxylic acid in step c).

MS: 369.1 (M−H)⁻

### EXAMPLE C27

### 5-Cyclohexylmethyl-3-(3,3-diphenyl-propionyl)-4-hydroxy-5H-furan-2-one

The title compound was obtained in comparable yields according to the procedures described for example C1 using 3,3-diphenyl-propionic acid (commercially available) instead of cyclohexanecarboxylic acid in step c).

MS: 403.3 (M−H)⁻

### EXAMPLE C28

### 5-Cyclohexylmethyl-3-(2,3-diphenyl-propionyl)-4-hydroxy-5H-furan-2-one

The title compound was obtained in comparable yields according to the procedures described for example C1 using 2,3-diphenyl-propionic acid (commercially available) instead of cyclohexanecarboxylic acid in step c).

MS: 403.3 (M−H)⁻

### EXAMPLE C29

### 5-Cyclohexylmethyl-3-[3-(4-fluoro-phenyl)-2-phenyl-propionyl]-4-hydroxy-5H-furan-2-one

The title compound was obtained in comparable yields according to the procedures described for example C1 using 3-(4-fluoro-phenyl)-2-phenyl-propionic acid (commercially available) instead of cyclohexanecarboxylic acid in step c).

MS: 421.1 (M−H)⁻

### EXAMPLE C30

### 3-(2-Benzyl-3-phenyl-propionyl)-5-cyclohexylmethyl-4-hydroxy-5H-furan-2-one

The title compound was obtained in comparable yields according to the procedures described for example C1 using 2-benzyl-3-phenyl-propionic acid (commercially available) instead of cyclohexanecarboxylic acid in step c).

MS: 417.2 (M−H)⁻

### EXAMPLE C31

### 3-[2-(4-Chloro-benzyl)-3-(4-chloro-phenyl)-propionyl]-5-cyclohexylmethyl-4-hydroxy-5H-furan-2-one

The title compound was obtained in comparable yields according to the procedures described for example C1 using 2-(4-chloro-benzyl)-3-(4-chloro-phenyl)-propionic acid (prepared according to Iizuka, Kinji; Kamijo, Tetsuhide; Kubota, Tetsuhiro; Akahane, Kenji; Umeyama, Hideaki; Kiso, Yoshiaki. EP252727 A1 (1988), 21 pp.) instead of cyclohexanecarboxylic acid in step c).

MS: 485.2 (M−H)⁻

### EXAMPLE C32

### 5-Cyclohexylmethyl-3-[(9H-fluoren-9-yl)-acetyl]-4-hydroxy-5H-furan-2-one

The title compound was obtained in comparable yields according to the procedures described for example C1 using (9H-fluoren-9-yl)-acetic acid (commercially available) instead of cyclohexanecarboxylic acid in step c).

MS: 401.4 (M−H)⁻

### EXAMPLE C33

### 3-(Carbazol-9-yl-acetyl)-5-cyclohexylmethyl-4-hydroxy-5H-furan-2-one

The title compound was obtained in comparable yields according to the procedures described for example C1 using Carbazol-9-yl-acetic acid (commercially available) instead of cyclohexanecarboxylic acid in step c).

MS: 402.3 (M−H)⁻

¹H-NMR (300 MHz, internal standard TMS, J values in Hz, d6-DMSO): 8.13 (d, J=7.1, 2H), 7.26 (s, br. 4H), 7.20-7.10 (m, 2H), 5.49 (s, br. 2H), 4.33 (dd, J=9.8 and 2.8, 1H), 3.0 (s, br., 1H), 1.90-0.80 (m, 13H)

### EXAMPLE D1

### 5-Benzyl-3-cyclohexanecarbonyl-4-hydroxy-5H-furan-2-one

### a) 5-Benzyl-4-methoxy-5H-furan-2-one

To a solution of 20 ml of LDA (2M in THF) and 130 ml of THF was added at −95° C. to −100° C. a solution of 5.47 g of 3(E)-methoxy-acrylic acid methyl ester in 4.5 ml of THF within 1 min, stirring was continued at the same temperature for 5 min, which was followed by the addition of a pre-cooled (−78° C.) solution of 33 mmole of the phenyl-acetaldehyde in 4.5 ml of THF within 2 min and stirring was continued at −100° C. for 30 min and at −78° C. for 1 h. The cold solution was poured onto 130 ml of ice-water, the pH was adjusted to 4 with 6.5 ml of aqueous HCl (37%) and the layers were separated. The aqueous layer was extracted twice with dichloromethane, the organic layers were washed with brine, dried and evaporated. The residue was chromatographed on silica (n-heptane/AcOEt, various ratios) to give the 5-benzyl-4-methoxy-5H-furan-2-one in 30-40% yield.

MS: 205.2 (M+H)⁺

### b) 5-Benzyl-4-hydroxy-5H-furan-2-one

A mixture of the 5-benzyl-4-methoxy-5H-furan-2-one (10 mmole) and 15 ml of aqueous HCl (37%) was stirred at 40° C. until completion of the reaction. The suspension was filtered and the residue washed with ice-cold water and dried. An oily reaction mixture was extracted with dichloromethane, the organic layers were washed with brine, dried and evaporated. The residue was either triturated with AcOEt/hexane or chromatographed with dichloromethane/MeOH (various ratios) to give 5-benzyl-4-hydroxy-5H-furan-2-one in 60-90% yield.

MS: 190.1 (M)⁺

### 5-Benzyl-3-cyclohexanecarbonyl-4-hydroxy-5H-furan-2-one

To as suspension of the 5-benzyl-4-hydroxy-5H-furan-2-one (0.2 mmole), NEt₃ (0.68 mmole), DMAP (0.066 mmole) and EDC (0.44 mmole) in 2 ml of THF was added at 22° C. cyclohexanecarboxylic acid (0.22 mmole) (commercially available) and stirring was continued until completion of the reaction. The pH of the reaction mixture was adjusted to 3 using aqueous HCl (2 N), the aqueous solution was saturated with NaCl, the organic layer was separated, washed with brine dried and evaporated. The residue was purified on preparative HPLC (RP-18, CH₃CN/H₂O, gradient) to give the 5-Benzyl-3-cyclohexanecarbonyl-4-hydroxy-5H-furan-2-one in 10-60% yield.

MS: 299.2 (M−H)⁻

### EXAMPLE D2

### 5-Benzyl-3-[3-(4-tert-butyl-phenyl)-2-methyl-propionyl]-4-hydroxy-5H-furan-2-one

The title compound was obtained in comparable yields according to the procedures described for example D1 using 3-(4-tert-butyl-phenyl)-2-methyl-propionic acid (prepared according to Kuchar, Miroslav; Rejholec, Vaclav; Roubal, Zdenek; Nemecek, Oldrich; Collect. Czech. Chem. Commun. (1979), 44(1), 183-93) instead of cyclohexanecarboxylic acid in step c).

MS: 391.1 (M−H)⁻

### EXAMPLE D3

### 5-Benzyl-4-hydroxy-3-[(2-methoxy-phenoxy)-acetyl]-5H-furan-2-one

The title compound was obtained in comparable yields according to the procedures described for example D1 using (2-methoxy-phenoxy)-acetic acid (commercially available) instead of cyclohexanecarboxylic acid in step c).

MS: 353.1 (M−H)⁻

### EXAMPLE D4

### 5-Benzyl-3-(4-cyclohexyl-butyryl)-4-hydroxy-5H-furan-2-one

The title compound was obtained in comparable yields according to the procedures described for example D1 using 4-cyclohexyl-butyric acid (commercially available) instead of cyclohexanecarboxylic acid in step c).

MS: 341.1 (M−H)⁻

### EXAMPLE D5

### 5-Benzyl-4-hydroxy-3-[(H-indol-3-yl)-acetyl]-5H-furan-2-one

The title compound was obtained in comparable yields according to the procedures described for example D1 using (1H-indol-3-yl)-acetic acid (commercially available) instead of cyclohexanecarboxylic acid in step c).

MS: 346.1 (M−H)⁻

### EXAMPLE D6

### 5-Benzyl-3-(3,3-diphenyl-propionyl)-4-hydroxy-5H-furan-2-one

The title compound was obtained in comparable yields according to the procedures described for example D1 using 3,3-diphenyl-propionic acid (commercially available) instead of cyclohexanecarboxylic acid in step c).

MS: 397.2 (M−H)⁻

### EXAMPLE D7

### 5-Benzyl-3-[(9H-fluoren-9-yl)-acetyl]-4-hydroxy-5H-furan-2-one

The title compound was obtained in comparable yields according to the procedures described for example D1 using (9H-fluoren-9-yl)-acetic acid (commercially available) instead of cyclohexanecarboxylic acid in step c).

MS: 395.1 (M−H)⁻

### EXAMPLE E1

### Rac-4-Hydroxy-3-(3-methyl-sulfanyl-propionyl)-5-phenethyl-5H-furan-2-one

### a) 4-Hydroxy-5-phenethyl-5H-furan-2-one

To a solution of 20 ml of LDA (2M in THF) and 130 ml of THF was added at −95° C. to −100° C. a solution of 5.47 g of 3(E)-methoxy-acrylic acid methyl ester in 4.5 ml of THF within 1 min, stirring was continued at the same temperature for 5 min, which was followed by the addition of a pre-cooled (−78° C.) solution of 33 mmole of the 3-phenyl-propionaldehyde in 4.5 ml of THF within 2 min and stirring was continued at −100° C. for 30 min and at −78° C. for 1 h. The cold solution was poured onto 130 ml of ice-water, the pH was adjusted to 4 with 6.5 ml of aqueous HCl (37%) and the layers were separated. The aqueous layer was extracted twice with dichloromethane, the organic layers were washed with brine, dried and evaporated. The residue was chromatographed on silica (n-heptane/AcOEt, various ratios) to give the 4-hydroxy-5-phenethyl-5H-furan-2-one in 30-40% yield.

MS: 218.0 (M)⁺

### b) 4-Hydroxy-5-phenethyl-5H-furan-2-one

A mixture of the 4-hydroxy-5-phenethyl-5H-furan-2-one (10 mmole) and 15 ml of aqueous HCl (37%) was stirred at 40° C. until completion of the reaction. The suspension was filtered and the residue washed with ice-cold water and dried. An oily reaction mixture was extracted with dichloromethane, the organic layers were washed with brine, dried and evaporated. The residue was either triturated with AcOEt/hexane or chromatographed with dichloromethane/MeOH (various ratios) to give 4-hydroxy-5-phenethyl-5H-furan-2-one in 60-90% yield.

MS: 202.9 (M−H)⁻

### c) Rac-4-Hydroxy-3-(3-methyl-sulfanyl-propionyl)-5-phenethyl-5H-furan-2-one

To as suspension of the 4-hydroxy-5-phenethyl-5H-furan-2-one (0.2 mmole), NEt₃ (0.68 mmole), DMAP (0.066 mmole) and EDC (0.44 mmole) in 2 ml of THF was added at 22° C. 3-methyl-sulfanyl-propionic acid (0.22 mmole) (commercially available) and stirring was continued until completion of the reaction. The pH of the reaction mixture was adjusted to 3 using aqueous HCl (2 N), the aqueous solution was saturated with NaCl, the organic layer was separated, washed with brine dried and evaporated. The residue was purified on preparative HPLC (RP-18, CH₃CN/H₂O, gradient) to give the Rac-4-hydroxy-3-(3-methyl-sulfanyl-propionyl)-5-phenethyl-5H-furan-2-one in 10-60% yield.

MS: 305.0 (M−H)⁻

### EXAMPLE E2

### Rac-3-(2(R,S),4-dimethyl-pentanoyl)-4-hydroxy-5-phenethyl-5H-furan-2-one

The title compound was obtained in comparable yields according to the procedures described for example E1 using 2(R,S),4-dimethyl-pentanoic acid (commercially available) instead of 3-methyl-sulfanyl-propionic acid in step c).

MS: 315.2 (M−H)⁻

### EXAMPLE E3

### Rac-4-hydroxy-3-(2(R,S)-methyl-hexanoyl)-5-phenethyl-5H-furan-2-one

The title compound was obtained in comparable yields according to the procedures described for example E1 using 2(R,S),4-dimethyl-pentanoic acid (commercially available) instead of 3-methyl-sulfanyl-propionic acid in step c).

MS: 315.2 (M−H)⁻

### EXAMPLE E4

### Rac-3-cyclopropane-carbonyl-4-hydroxy-5-phenethyl-5H-furan-2-one

The title compound was obtained in comparable yields according to the procedures described for example E1 using 3-cyclopropane-carboxylic acid (commercially available) instead of 3-methyl-sulfanyl-propionic acid in step c).

MS: 271.2 (M−H)⁻

### EXAMPLE E5

### Rac-3-cyclohexane-carbonyl-4-hydroxy-5-phenethyl-5H-furan-2-one

The title compound was obtained in comparable yields according to the procedures described for example E1 using cyclohexane-carboxylic acid (commercially available) instead of 3-methyl-sulfanyl-propionic acid in step c).

MS: 210.1 (M−C₈H₈)⁺

### EXAMPLE E6

### Rac-3-(2-cyclohexyl-acetyl)-4-hydroxy-5-phenethyl-5H-furan-2-one

The title compound was obtained in comparable yields according to the procedures described for example E1 using 2-cyclohexyl-acetic acid (commercially available) instead of 3-methyl-sulfanyl-propionic acid in step c).

MS: 327.2 (M−H)⁻

### EXAMPLE E7

### Rac-3-(4-cyclohexyl-butyryl)-4-hydroxy-5-phenethyl-5H-furan-2-one

The title compound was obtained in comparable yields according to the procedures described for example E1 using 4-cyclohexyl-butyric acid (commercially available) instead of 3-methyl-sulfanyl-propionic acid in step c).

MS: 355.2 (M−H)⁻

### EXAMPLE E8

Rac-4-hydroxy-5-phenethyl-3-phenylacetyl-5H-furan-2-one

The title compound was obtained in comparable yields according to the procedures described for example E1 using phenylacetic acid (commercially available) instead of 3-methyl-sulfanyl-propionic acid in step c).

MS: 321.1 (M−H)⁻

### EXAMPLE E9

### Rac-4-hydroxy-5-phenethyl-3-(2-o-tolyl-acetyl)-5H-furan-2-one

The title compound was obtained in comparable yields according to the procedures described for example E1 using 2-o-tolyl-acetic acid (commercially available) instead of 3-methyl-sulfanyl-propionic acid in step c).

MS: 335.1 (M−H)⁻

### EXAMPLE E10

### Rac-4-hydroxy-5-phenethyl-3-(2(R,S)-phenyl-propionyl)-5H-furan-2-one

The title compound was obtained in comparable yields according to the procedures described for example E1 using 2(R,S)-phenyl-propionic acid (commercially available) instead of 3-methyl-sulfanyl-propionic acid in step c).

MS: 335.0 (M−H)⁻

### EXAMPLE E11

### Rac-4-hydroxy-5-phenethyl-3-(2(R,S)-phenyl-butyryl)-5H-furan-2-one

The title compound was obtained in comparable yields according to the procedures described for example E1 using 2(R,S)-phenyl-butyric acid (commercially available) instead of 3-methyl-sulfanyl-propionic acid in step c).

MS: 349.2 (M−H)⁻

### EXAMPLE E12

### Rac-3-[2-(2,5-dimethoxy-phenyl)-acetyl]-4-hydroxy-5-phenethyl-5H-furan-2-one

The title compound was obtained in comparable yields according to the procedures described for example E1 using 2-(2,5-dimethoxy-phenic acid (commercially available) instead of 3-methyl-sulfanyl-propionic acid in step c).

MS: 381.2 (M−H)⁻

### EXAMPLE E13

### Rac-3-[2-(2,4-dimethoxy-phenyl)-acetyl]-4-hydroxy-5-phenethyl-5H-furan-2-one

The title compound was obtained in comparable yields according to the procedures described for example E1 using 2-(2,4-dimethoxy-phenyl)-acetic acid (commercially available) instead of 3-methyl-sulfanyl-propionic acid in step c).

MS: 381.1 (M−H)⁻

### EXAMPLE E14

### Rac-3-[2-(3,5-dimethoxy-phenyl)-acetyl]-4-hydroxy-5-phenethyl-5H-furan-2-one

The title compound was obtained in comparable yields according to the procedures described for example E1 using 2-(3,5-dimethoxy-phenyl)-acetic acid (commercially available) instead of 3-methyl-sulfanyl-propionic acid in step c).

MS: 381.1 (M−H)⁻

### EXAMPLE E15

### Rac-4-hydroxy-5-phenethyl-3-(3-phenyl-propionyl)-5H-furan-2-one

The title compound was obtained in comparable yields according to the procedures described for example E1 using 3-phenyl-propionic acid (commercially available) instead of 3-methyl-sulfanyl-propionic acid in step c).

MS: 335.1 (M−H)⁻

### EXAMPLE E16

### 4-Hydroxy-5-phenethyl-3-((R)—(R)-2-phenyl-cyclopropanecarbonyl)-5H-furan-2-one

The title compound was obtained in comparable yields according to the procedures described for example E1 using (R)—(R)-2-phenyl-cyclopropanecarboxylic acid (commercially available) instead of 3-methyl-sulfanyl-propionic acid in step c).

MS: 347.2 (M−H)⁻

### EXAMPLE E17

### Rac-4-hydroxy-5-phenethyl-3-(3(R,S)-phenyl-butyryl)-5H-furan-2-one

The title compound was obtained in comparable yields according to the procedures described for example E1 using 3(R,S)-phenyl-butyric acid (commercially available) instead of 3-methyl-sulfanyl-propionic acid in step c).

MS: 349.2 (M−H)⁻

### EXAMPLE E18

### Rac-4-hydroxy-3-(2(R,S)-hydroxy-3-phenyl-propionyl)-5-phenethyl-5H-furan-2-one

The title compound was obtained in comparable yields according to the procedures described for example E1 using 2(R,S)-hydroxy-3-phenyl-propionic acid (commercially available) instead of 3-methyl-sulfanyl-propionic acid in step c).

MS: 351.1 (M−H)⁻

### EXAMPLE E19

### Rac-4-hydroxy-5-phenethyl-3-(3-m-tolyl-propionyl)-5H-furan-2-one

The title compound was obtained in comparable yields according to the procedures described for example E1 using 3-m-tolyl-propionic acid (commercially available) instead of 3-methyl-sulfanyl-propionic acid in step c).

MS: 349.3 (M−H)⁻

### EXAMPLE E20

### Rac-4-hydroxy-3-[2-(2-methoxy-phenoxy)-acetyl]-5-phenethyl-5H-furan-2-one

The title compound was obtained in comparable yields according to the procedures described for example E1 using 2-(2-methoxy-phenoxy)-acetic acid (commercially available) instead of 3-methyl-sulfanyl-propionic acid in step c).

MS: 369.2 (M+H)⁺

### EXAMPLE E21

### Rac-4-hydroxy-3-[3-(3-methoxy-phenyl)-propionyl]-5-phenethyl-5H-furan-2-one

The title compound was obtained in comparable yields according to the procedures described for example E1 using 3-(3-methoxy-phenyl)-propionic acid (commercially available) instead of 3-methyl-sulfanyl-propionic acid in step c).

MS: 365.1 (M−H)⁻

### EXAMPLE E22

### Rac-4-hydroxy-3-[3-(4-methoxy-phenyl)-propionyl]-5-phenethyl-5H-furan-2-one

The title compound was obtained in comparable yields according to the procedures described for example E1 using 3-(4-methoxy-phenyl)-propionic acid (commercially available) instead of 3-methyl-sulfanyl-propionic acid in step c).

MS: 365.0 (M−H)⁻

### EXAMPLE E23

### Rac-3-[3-(2,5-dimethoxy-phenyl)-propionyl]-4-hydroxy-5-phenethyl-5H-furan-2-one

The title compound was obtained in comparable yields according to the procedures described for example E1 using 3-(2,5-dimethoxy-phenyl)-propionic acid (commercially available) instead of 3-methyl-sulfanyl-propionic acid in step c).

MS: 395.2 (M−H)⁻

### EXAMPLE E24

### Rac-3-[3-(4-tert-butyl-phenyl)-2(R,S)-methyl-propionyl]-4-hydroxy-5-phenethyl-5H-furan-2-one

The title compound was obtained in comparable yields according to the procedures described for example E1 using 3-(4-tert-butyl-phenyl)-2(R,S)-methyl-propionic acid (prepared according to Kuchar, Miroslav; Rejholec, Vaclav; Roubal, Zdenek; Nemecek, Oldrich; Collect. Czech. Chem. Commun. (1979), 44(1), 183-93) instead of 3-methyl-sulfanyl-propionic acid in step c).

MS: 405.4 (M−H)⁻

### EXAMPLE E25

### Rac-3-[3-(4-chloro-phenyl)-2(R,S)-methyl-propionyl]-4-hydroxy-5-phenethyl-5H-furan-2-one

The title compound was obtained in comparable yields according to the procedures described for example E1 using 3-(4-chloro-phenyl)-2(R,S)-methyl-propionic acid (prepared according to Ferorelli, S.; Loiodice, F.; Tortorella, V.; Amoroso, R.; Bettoni, G.; Conte-Camerino, D.; De Luca, A.; Farmaco (1997), 52(6-7), 367-374) instead of 3-methyl-sulfanyl-propionic acid in step c).

MS: 383.1 (M−H)⁻

### EXAMPLE E26

### 4-Hydroxy-5-phenethyl-3-(4-phenyl-butyryl)-5H-furan-2-one

The title compound was obtained in comparable yields according to the procedures described for example E1 using 4-phenyl-butyric acid (commercially available) instead of 3-methyl-sulfanyl-propionic acid in step c).

MS: 349.3 (M−H)⁻

### EXAMPLE E27

### 3-[4-(3,4-Dimethoxy-phenyl)-butyryl]-4-hydroxy-5-phenethyl-5H-furan-2-one

The title compound was obtained in comparable yields according to the procedures described for example E1 using 4-(3,4-Dimethoxy-phenyl)-butyric acid (commercially available) instead of 3-methyl-sulfanyl-propionic acid in step c).

MS: 409.2 (M−H)⁻

### EXAMPLE E28

### 4-Hydroxy-3-(2-naphthalen-2-yl-acetyl)-5-phenethyl-5H-furan-2-one

The title compound was obtained in comparable yields according to the procedures described for example E1 using 2-naphthalen-2-yl-acetic acid (commercially available) instead of 3-methyl-sulfanyl-propionic acid in step c).

MS: 371.1 (M−H)⁻

### EXAMPLE E29

### Rac-4-hydroxy-3-[2 (R,S)-(6-methoxy-naphthalen-2-yl)-propionyl]-5-phenethyl-5H-furan-2-one

The title compound was obtained in comparable yields according to the procedures described for example E1 using 2(R,S)-(6-methoxy-naphthalen-2-yl)-propionic acid (commercially available) instead of 3-methyl-sulfanyl-propionic acid in step c).

MS: 415.2 (M−H)⁻

### EXAMPLE E30

### 3-[(2-Acetyl-naphthalen-1-yl)-acetyl]-4-hydroxy-5-phenethyl-5H-furan-2-one

The title compound was obtained in comparable yields according to the procedures described for example E1 using (2-Acetyl-naphthalen-1-yl)-acetic acid (commercially available) instead of 3-methyl-sulfanyl-propionic acid in step c).

MS: 415.2 (M−H)⁻

### EXAMPLE E31

### 3-[2-(2-Acetyl-1,2-dihydro-isoquinolin-1-yl)-acetyl]-4-hydroxy-5-phenethyl-5H-furan-2-one

The title compound was obtained in comparable yields according to the procedures described for example E1 using 2-(2-Acetyl-1,2-dihydro-isoquinolin-1-yl)-acetic acid (commercially available) instead of 3-methyl-sulfanyl-propionic acid in step c).

MS: 416.1 (M−H)⁻

### EXAMPLE E32

### 4-Hydroxy-3-(2-1H-indol-3-yl-acetyl)-5-phenethyl-5H-furan-2-one

The title compound was obtained in comparable yields according to the procedures described for example E1 using 2-1H-indol-3-yl-acetic acid (commercially available) instead of 3-methyl-sulfanyl-propionic acid in step c).

MS: 360.0 (M−H)⁻

### EXAMPLE E33

### Rac-4-hydroxy-3-(3-1H-indol-3-yl-propionyl)-5-phenethyl-5H-furan-2-one

The title compound was obtained in comparable yields according to the procedures described for example E1 using 3-1H-indol-3-yl-propionic acid (commercially available) instead of 3-methyl-sulfanyl-propionic acid in step c).

MS: 374.2 (M−H)⁻

### EXAMPLE E34

### Rac-4-hydroxy-3-[2-(naphthalen-1-yloxy)-acetyl]-5-phenethyl-5H-furan-2-one

The title compound was obtained in comparable yields according to the procedures described for example E1 using 2-(naphthalen-1-yloxy)-acetic acid (commercially available) instead of 3-methyl-sulfanyl-propionic acid in step c).

MS: 387.1 (M−H)⁻

### EXAMPLE E35

### Rac-3-(3,3-diphenyl-propionyl)-4-hydroxy-5-phenethyl-5H-furan-2-one

The title compound was obtained in comparable yields according to the procedures described for example E1 using 3,3-diphenyl-propionic acid (commercially available) instead of 3-methyl-sulfanyl-propionic acid in step c).

MS: 411.2 (M−H)⁻

### EXAMPLE E36

### Rac-3-(2-10,11-dihydro-5H-dibenzo[a,d]cyclohepten-5-yl-acetyl)-4-hydroxy-5-phenethyl-5H-furan-2-one

The title compound was obtained in comparable yields according to the procedures described for example E1 using 2-10,11-dihydro-5H-dibenzo[a,d]cyclohepten-5-yl-acetic acid (prepared according to Tucker, Thomas J.; Lumma, William C.; Lewis, S. Dale; Gardell, Stephen J.; Lucas, Bobby J.; Sisko, Jack T.; Lynch, Joseph J.; Lyle, Elizabeth A.; Baskin, Elizabeth P.; Woltmann, Richard F.; Appleby, Sandra D.; Chen, I-Wu; Dancheck, Kimberley B.; Naylor-Olsen, Adel M.; Krueger, Julie A.; Cooper, Carolyn M.; Vacca, Joseph P. Journal of Medicinal Chemistry (1997), 40(22), 3687-3693) instead of 3-methyl-sulfanyl-propionic acid in step c).

MS: 437.3 (M−H)⁻

### EXAMPLE E37

### Rac-4-hydroxy-5-phenethyl-3-(2-9H-thioxanthen-9-yl-acetyl)-5H-furan-2-one

The title compound was obtained in comparable yields according to the procedures described for example E1 using 2-9H-thioxanthen-9-yl-acetic acid (prepared according to Jilek, Jiri O.; Holubek, Jiri; Svatek, Emil; Ryska, Miroslav; Pomykacek, Josef; Protiva, Miroslav. Collection of Czechoslovak Chemical Communications (1979), 44(7), 2124-38) instead of 3-methyl-sulfanyl-propionic acid in step c).

MS: 441.6 (M−H)⁻

### EXAMPLE E38

Rac-3-(2-9H-fluoren-9-yl-acetyl)-4-hydroxy-5-phenethyl-5H-furan-2-one

The title compound was obtained in comparable yields according to the procedures described for example E1 using 2-9H-fluoren-9-yl-acetic acid (commercially available) instead of 3-methyl-sulfanyl-propionic acid in step c).

MS: 409.2 (M−H)⁻

### EXAMPLE E39

### Rac-[2-(4-hydroxy-2-oxo-5-phenethyl-2,5-dihydro-furan-3-yl)-1(R,S)-methyl-2-oxo-ethyl]-carbamic acid tert-butyl ester

The title compound was obtained in comparable yields according to the procedures described for example E1 using

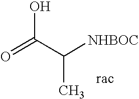

(commercially available) instead of 3-methyl-sulfanyl-propionic acid in step c).

MS: 374.2 (M−H)⁻

### EXAMPLE E40

### Rac-3-(2(R,S)-amino-propionyl)-4-hydroxy-5-phenethyl-5H-furan-2-one

The title compound was prepared from the corresponding BOC-protected precursor (Example E40) by deprotection using CF₃COOH.

MS: 276.1 (M+H)⁺

### EXAMPLE E41

### [1(R)-Benzyl-2-(4-hydroxy-2-oxo-5(R,S)-phenethyl-2,5-di-hydro-furan-3-yl)-2-oxo-ethyl]-carbamic acid tert-butylester

The title compound was obtained in comparable yields according to the procedures described for example E1 using

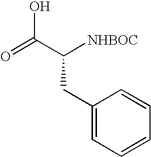

(commercially available) instead of 3-methyl-sulfanyl-propionic acid in step c).

MS: 450.1 (M−H)⁻

### EXAMPLE E42

### 3-(2(R)-Amino-3-phenyl-propionyl)-4-hydroxy-5(R,S)-phenethyl-5H-furan-2-one

The title compound was prepared from the corresponding BOC-protected precursor (Example E42) by deprotection using CF₃COOH.

MS: 352.2 (M+H)⁺

### EXAMPLE E43

### Rac-[1(R,S)-(4-benzyloxy-benzyl)-2-(4-hydroxy-2-oxo-5-phenethyl-2,5-dihydro-furan-3-yl)-2-oxo-ethyl]-carbamic acid tert-butyl ester

The title compound was obtained in comparable yields according to the procedures described for example E1 using

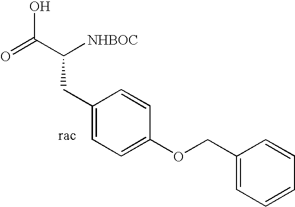

(commercially available) instead of 3-methyl-sulfanyl-propionic acid in step c).

MS: 556.2 (M−H)⁻

### EXAMPLE E44

### [1(S)-(4-Benzyloxy-benzyl)-2-(4-hydroxy-2-oxo-5(R,S)-phenethyl-2,5-dihydro-furan-3-yl)-2-oxo-ethyl]-carbamic acid tert-butyl ester

The title compound was obtained in comparable yields according to the procedures described for example E1 using

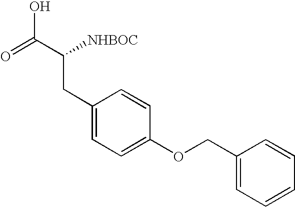

(commercially available) instead of 3-methyl-sulfanyl-propionic acid in step c).

MS: 458.2 (M+H−C₅H₉O₂)⁺

### EXAMPLE E45

### [1(R)-(4-Benzyloxy-benzyl)-2-(4-hydroxy-2-oxo-5(R,S)-phenethyl-2,5-dihydro-furan-3-yl)-2-oxo-ethyl]-carbamic acid tert-butyl ester

The title compound was obtained in comparable yields according to the procedures described for example E1 using

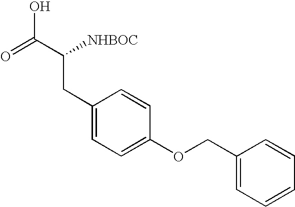

(commercially available) instead of 3-methyl-sulfanyl-propionic acid in step c).

MS: 458.2 (M+H−C₅H₉O₂)⁺

### EXAMPLE E46

### Rac-3-[2(R,S)-amino-3-(4-benzyloxy-phenyl)-propionyl]-4-hydroxy-5-phenethyl-5H-furan-2-one

The title compound was prepared from the corresponding BOC-protected precursor (Example E44) by deprotection using CF₃COOH.

MS: 458.3 (M+H)⁺

### EXAMPLE E47

### 2-(4-Hydroxy-2-oxo-5(R,S)-phenethyl-2,5-dihydro-furan-3-carbonyl)-pyrrolidine-1(S)-carboxylic acid tert-butyl ester

The title compound was obtained in comparable yields according to the procedures described for example E1 using

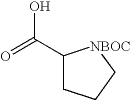

(commercially available) instead of 3-methyl-sulfanyl-propionic acid in step c).

MS: 400.3 (M−H)⁻

### EXAMPLE E48

### 4-Hydroxy-5(R,S)-phenethyl-3-(pyrrolidine-2(S)-carbonyl)-5H-furan-2-one

The title compound was prepared from the corresponding BOC-protected precursor (Example E48) by deprotection using CF₃COOH.

MS: 302.1 (M+H)⁺

### EXAMPLE E49

### Rac-2(R,S)-(4-Hydroxy-2-oxo-5-phenethyl-2,5-dihydro-furan-3-carbonyl)-piperidine-1-carboxylic acid tert-butyl ester

The title compound was obtained in comparable yields according to the procedures described for example E1 using

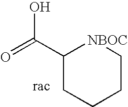

(commercially available) instead of 3-methyl-sulfanyl-propionic acid in step c).

MS: 414.2 (M−H)⁻

### EXAMPLE E50

### Rac-4-hydroxy-5-phenethyl-3(R,S)-(piperidine-2-carbonyl)-5H-furan-2-one

The title compound was prepared from the corresponding BOC-protected precursor (Example E50) by deprotection using CF₃COOH.

MS: 316.1 (M+H)⁺

### EXAMPLE E51

### Rac-3(R,S)-(4-hydroxy-2-oxo-5-phenethyl-2,5-dihydro-furan-3-carbonyl)-3,4-dihydro-1H-iso-quinoline-2-carboxylic acid tert-butyl ester

The title compound was obtained in comparable yields according to the procedures described for example E1 using

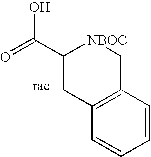

(commercially available) instead of 3-methyl-sulfanyl-propionic acid in step c).

MS: 462.2 (M−H)⁻

### EXAMPLE E52

### Rac-4-hydroxy-5-phenethyl-3(R,S)-(1,2,3,4-tetrahydro-isoquinoline-3-carbonyl)-5H-furan-2-one

The title compound was prepared from the corresponding BOC-protected precursor (Example E52) by deprotection using CF₃COOH.

MS: 364.1 (M+H)⁺

### EXAMPLE F1

### 3-4-Cyclohexanecarbonyl-4-hydroxy-5-(3-phenyl-propyl)-5H-furan-2-one

### a) 4-Methoxy-5-(3-phenyl-propyl)-5H-furan-2-one

To a solution of 20 ml of LDA (2M in THF) and 130 ml of THF was added at −95° C. to −100° C. a solution of 5.47 g of 3(E)-methoxy-acrylic acid methyl ester in 4.5 ml of THF within 1 min, stirring was continued at the same temperature for 5 min, which was followed by the addition of a pre-cooled (−78° C.) solution of 33 mmole of the 4-phenyl-butyraldehyde in 4.5 ml of THF within 2 min and stirring was continued at −100° C. for 30 min and at −78° C. for 1 h. The cold solution was poured onto 130 ml of ice-water, the pH was adjusted to 4 with 6.5 ml of aqueous HCl (37%) and the layers were separated. The aqueous layer was extracted twice with dichloromethane, the organic layers were washed with brine, dried and evaporated. The residue was chromatographed on silica (n-heptane/AcOEt, various ratios) to give the 4-methoxy-5-(3-phenyl-propyl)-5H-furan-2-one in 30-40% yield.

MS: 250.3 (M+NH₄)⁺

### b) 4-Hydroxy-5-(3-phenyl-propyl)-5H-furan-2-one

A mixture of the 4-methoxy-5-(3-phenyl-propyl)-5H-furan-2-one (10 mmole) and 15 ml of aqueous HCl (37%) was stirred at 40° C. until completion of the reaction. The suspension was filtered and the residue washed with ice-cold water and dried. An oily reaction mixture was extracted with dichloromethane, the organic layers were washed with brine, dried and evaporated. The residue was either triturated with AcOEt/hexane or chromatographed with dichloromethane/MeOH (various ratios) to give 4-hydroxy-5-(3-phenyl-propyl)-5H-furan-2-one in 60-90% yield.

MS: 218.1 (M)⁺

### c) 4-Cyclohexanecarbonyl-4-hydroxy-5-(3-phenyl-propyl)-5H-furan-2-one

To as suspension of the 4-hydroxy-5-(3-phenyl-propyl)-5H-furan-2-one (0.2 mmole), NEt₃ (0.68 mmole), DMAP (0.066 mmole) and EDC (0.44 mmole) in 2 ml of THF was added at 22° C. cyclohexanecarboxylic acid (0.22 mmole) (commercially available) and stirring was continued until completion of the reaction. The pH of the reaction mixture was adjusted to 3 using aqueous HCl (2 N), the aqueous solution was saturated with NaCl, the organic layer was separated, washed with brine dried and evaporated. The residue was purified on preparative HPLC (RP-18, CH₃CN/H₂O, gradient) to give the 3-4-Cyclohexanecarbonyl-4-hydroxy-5-(3-phenyl-propyl)-5H-furan-2-one in 10-60% yield.

MS: 327.2 (M−H)⁻

### EXAMPLE F2

### 3-(4-Cyclohexyl-butyryl)-4-hydroxy-5-(3-phenyl-propyl)-5H-furan-2-one

The title compound was obtained in comparable yields according to the procedures described for example F1 using 4-cyclohexyl-butyric acid (commercially available) instead of cyclohexanecarboxylic acid in step c).

MS: 369.1 (M−H)⁻

### EXAMPLE F3

### 3-[3-(4-tert-Butyl-phenyl)-2-methyl-propionyl]-4-hydroxy-5-(3-phenyl-propyl)-5H-furan-2-one

The title compound was obtained in comparable yields according to the procedures described for example F1 using 3-(4-tert-Butyl-phenyl)-2-methyl-propionic acid (prepared according to Kuchar, Miroslav; Rejholec, Vaclav; Roubal, Zdenek; Nemecek, Oldrich; Collect. Czech. Chem. Commun. (1979), 44(1), 183-93) instead of cyclohexanecarboxylic acid in step c).

MS: 419.1 (M−H)⁻

### EXAMPLE F4

### 4-Hydroxy-3-[(2-methoxy-phenoxy)-acetyl]-5-(3-phenyl-propyl)-5H-furan-2-one

The title compound was obtained in comparable yields according to the procedures described for example F1 using (2-methoxy-phenoxy)-acetic acid (commercially available) instead of cyclohexanecarboxylic acid in step c).

MS: 381.1 (M−H)⁻

### EXAMPLE F5

### 4-Hydroxy-3-[(H-indol-3-yl)-acetyl]-5-(3-phenyl-propyl)-5H-furan-2-one

The title compound was obtained in comparable yields according to the procedures described for example F1 using (1H-indol-3-yl)-acetic acid (commercially available) instead of cyclohexanecarboxylic acid in step c).

MS: 374.2 (M−H)⁻

### EXAMPLE F6

### 3-(3,3-Diphenyl-propionyl)-4-hydroxy-5-(3-phenyl-propyl)-5H-furan-2-one

The title compound was obtained in comparable yields according to the procedures described for example F1 using 3,3-Diphenyl-propionic acid (commercially available) instead of cyclohexanecarboxylic acid in step c).

MS: 425.2 (M−H)⁻

### EXAMPLE F7

### 3-[(9H-Fluoren-9-yl)-acetyl]-4-hydroxy-5-(3-phenyl-propyl)-5H-furan-2-one

The title compound was obtained in comparable yields according to the procedures described for example F1 using (9H-Fluoren-9-yl)-acetic acid (commercially available) instead of cyclohexanecarboxylic acid in step c).

MS: 423.2 (M−H)⁻

### EXAMPLE G1

### 4-Hydroxy-3-(3-methylsulfanyl-propionyl)-5-(3-morpholin-4-yl-propyl)-5H-furan-2-one

### a) 4-Methoxy-5-(3-morpholin-4-yl-propyl)-5H-furan-2-one

To a solution of 20 ml of LDA (2M in THF) and 130 ml of THF was added at −95° C. to −100° C. a solution of 5.47 g of 3(E)-methoxy-acrylic acid methyl ester in 4.5 ml of THF within 1 min, stirring was continued at the same temperature for 5 min, which was followed by the addition of a pre-cooled (−78° C.) solution of 33 mmole of the 4-morpholin-4-yl-butyraldehyde in 4.5 ml of THF within 2 min and stirring was continued at −100° C. for 30 min and at −78° C. for 1 h. The cold solution was poured onto 130 ml of ice-water, the pH was adjusted to 4 with 6.5 ml of aqueous HCl (37%) and the layers were separated. The aqueous layer was extracted twice with dichloromethane, the organic layers were washed with brine, dried and evaporated. The residue was chromatographed on silica (n-heptane/AcOEt, various ratios) to give the 4-methoxy-5-(3-morpholin-4-yl-propyl)-5H-furan-2-one in 30-40% yield.

MS: 242.3 (M+H)⁺

### b) 4-Hydroxy-5-(3-phenyl-propyl)-5H-furan-2-one

A mixture of the 4-methoxy-5-(3-morpholin-4-yl-propyl)-5H-furan-2-one (10 mmole) and 15 ml of aqueous HCl (37%) was stirred at 40° C. until completion of the reaction. The suspension was filtered and the residue washed with ice-cold water and dried. An oily reaction mixture was extracted with dichloromethane, the organic layers were washed with brine, dried and evaporated. The residue was either triturated with AcOEt/hexane or chromatographed with dichloromethane/MeOH (various ratios) to give 4-hydroxy-5-(3-phenyl-propyl)-5H-furan-2-one in 60-90% yield.

MS: 226.0 (M−H)⁻

### c) 4-Hydroxy-3-(3-methylsulfanyl-propionyl)-5-(3-morpholin-4-yl-propyl)-5H-furan-2-one

To as suspension of the 4-hydroxy-5-(3-phenyl-propyl)-5H-furan-2-one (0.2 mmole), NEt₃ (0.68 mmole), DMAP (0.066 mmole) and EDC (0.44 mmole) in 2 ml of THF was added at 22° C. 3-methyl-sulfanyl-propionic acid (0.22 mmole) (commercially available) and stirring was continued until completion of the reaction. The pH of the reaction mixture was adjusted to 3 using aqueous HCl (2 N), the aqueous solution was saturated with NaCl, the organic layer was separated, washed with brine dried and evaporated. The residue was purified on preparative HPLC (RP-18, CH₃CN/H₂O, gradient) to give the 4-hydroxy-3-(3-methylsulfanyl-propionyl)-5-(3-morpholin-4-yl-propyl)-5H-furan-2-one in 10-60% yield.

MS: 328.1 (M−H)⁻

### EXAMPLE G2

### 3-Cyclopropanecarbonyl-4-hydroxy-5-(3-morpholin-4-yl-propyl)-5H-furan-2-one

The title compound was obtained in comparable yields according to the procedures described for example G1 using cyclopropanecarboxylic acid (commercially available) instead of 3-methyl-sulfanyl-propionic acid in step c).

MS: 294.2 (M−H)⁻

### EXAMPLE G3

### 4-Hydroxy-5-(3-morpholin-4-yl-propyl)-3-(2,2,3,3-tetramethyl-cyclopropanecarbonyl)-5H-furan-2-one

The title compound was obtained in comparable yields according to the procedures described for example G1 using 2,2,3,3-tetramethyl-cyclopropanecarboxylic acid (commercially available) instead of 3-methyl-sulfanyl-propionic acid in step c).

MS: 350.3 (M−H)⁻

### EXAMPLE G4

### 4-Hydroxy-5-(3-morpholin-4-yl-propyl)-3-(tetrahydro-furan-2-carbonyl)-5H-furan-2-one

The title compound was obtained in comparable yields according to the procedures described for example G1 using tetrahydro-furan-2-carboxylic acid (commercially available) instead of 3-methyl-sulfanyl-propionic acid in step c).

MS: 324.1 (M−H)⁻

### EXAMPLE G5

### 3-Cyclohexanecarbonyl-4-hydroxy-5-(3-morpholin-4-yl-propyl)-5H-furan-2-one

The title compound was obtained in comparable yields according to the procedures described for example G1 using cyclohexanecarboxylic acid (commercially available) instead of 3-methyl-sulfanyl-propionic acid in step c).

MS: 338.2 (M+H)⁺

### EXAMPLE G6

### 3-(2-Cyclohexyl-acetyl)-4-hydroxy-5-(3-morpholin-4-yl-propyl)-5H-furan-2-one

The title compound was obtained in comparable yields according to the procedures described for example G1 using 2-cyclohexyl-acetic acid (commercially available) instead of 3-methyl-sulfanyl-propionic acid in step c).

MS: 350.3 (M−H)⁻

### EXAMPLE G7

### 3-(4-Cyclohexyl-butyryl)-4-hydroxy-5-(3-morpholin-4-yl-propyl)-5H-furan-2-one

The title compound was obtained in comparable yields according to the procedures described for example G1 using 4-cyclohexyl-butyric acid (commercially available) instead of 3-methyl-sulfanyl-propionic acid in step c).

MS: 378.2 (M−H)⁻

### EXAMPLE G8

### 4-Hydroxy-5-(3-morpholin-4-yl-propyl)-3-phenylacetyl-5H-furan-2-one

The title compound was obtained in comparable yields according to the procedures described for example G1 using phenylacetic acid (commercially available) instead of 3-methyl-sulfanyl-propionic acid in step c).

MS: 344.2 (M−H)⁻

### EXAMPLE G9

### 4-Hydroxy-5-(3-morpholin-4-yl-propyl)-3-(2-phenyl-propionyl)-5H-furan-2-one

The title compound was obtained in comparable yields according to the procedures described for example G1 using 2-phenyl-propionic acid (commercially available) instead of 3-methyl-sulfanyl-propionic acid in step c).

MS: 358.1 (M−H)⁻

### EXAMPLE G10

### 3-[2-(3,5-Dimethoxy-phenyl)-acetyl]-4-hydroxy-5-(3-morpholin-4-yl-propyl)-5H-furan-2-one

The title compound was obtained in comparable yields according to the procedures described for example G1 using 2-(3,5-Dimethoxy-phenyl)-acetic acid (commercially available) instead of 3-methyl-sulfanyl-propionic acid in step c).

MS: 404.4 (M−H)⁻

### EXAMPLE G11

### 3-[2-(2,5-Dimethoxy-phenyl)-acetyl]-4-hydroxy-5-(3-morpholin-4-yl-propyl)-5H-furan-2-one

The title compound was obtained in comparable yields according to the procedures described for example G1 using 2-(2,5-dimethoxy-phenyl)-acetic acid (commercially available) instead of 3-methyl-sulfanyl-propionic acid in step c).

MS: 404.3 (M−H)⁻

### EXAMPLE G12

### 3-[2-(2,4-Dimethoxy-phenyl)-acetyl]-4-hydroxy-5-(3-morpholin-4-yl-propyl)-5H-furan-2-one

The title compound was obtained in comparable yields according to the procedures described for example G1 using 2-(2,4-dimethoxy-phenyl)-acetic acid (commercially available) instead of 3-methyl-sulfanyl-propionic acid in step c).

MS: 404.2 (M−H)⁻

### EXAMPLE G13

### 4-Hydroxy-3-[2-(4-methoxy-2-methyl-phenyl)-acetyl]-5-(3-morpholin-4-yl-propyl)-5H-furan-2-one

The title compound was obtained in comparable yields according to the procedures described for example G1 using 2-(4-methoxy-2-methyl-phenyl)-acetic acid (commercially available) instead of 3-methyl-sulfanyl-propionic acid in step c).

MS: 390.3 (M+H)⁺

### EXAMPLE G14

### 4-Hydroxy-3-[3-(4-methoxy-phenyl)-propionyl]-5-(3-morpholin-4-yl-propyl)-5H-furan-2-one

The title compound was obtained in comparable yields according to the procedures described for example G1 using 3-(4-methoxy-phenyl)-propionic acid (commercially available) instead of 3-methyl-sulfanyl-propionic acid in step c).

MS: 388.2 (M−H)⁻

### EXAMPLE G15

### 4-Hydroxy-5-(3-morpholin-4-yl-propyl)-3-(3-phenyl-butyryl)-5H-furan-2-one

The title compound was obtained in comparable yields according to the procedures described for example G1 using 3-phenyl-butyric acid (commercially available) instead of 3-methyl-sulfanyl-propionic acid in step c).

MS: 372.2 (M−H)⁻

### EXAMPLE G16

### 3-[3-(2,5-Dimethoxy-phenyl)-propionyl]-4-hydroxy-5-(3-morpholin-4-yl-propyl)-5H-furan-2-one

The title compound was obtained in comparable yields according to the procedures described for example G1 using 2,5-dimethoxy-phenyl)-propionic acid (commercially available) instead of 3-methyl-sulfanyl-propionic acid in step c).

MS: 418.2 (M−H)⁻

### EXAMPLE G17

### 4-Hydroxy-5-(3-morpholin-4-yl-propyl)-3-(3-m-tolyl-propionyl)-5H-furan-2-one

The title compound was obtained in comparable yields according to the procedures described for example G1 using 3-m-tolyl-propionic acid (commercially available) instead of 3-methyl-sulfanyl-propionic acid in step c).

MS: 372.2 (M−H)⁻

### EXAMPLE G18

### 4-Hydroxy-3-[3-(3-methoxy-phenyl)-propionyl]-5-(3-morpholin-4-yl-propyl)-5H-furan-2-one

The title compound was obtained in comparable yields according to the procedures described for example G1 using 3-(3-methoxy-phenyl)-propionic acid (commercially available) instead of 3-methyl-sulfanyl-propionic acid in step c).

MS: 388.1 (M−H)⁻

### EXAMPLE G19

### 4-Hydroxy-3-[2-(3-methoxy-phenoxy)-acetyl]-5-(3-morpholin-4-yl-propyl)-5H-furan-2-one

The title compound was obtained in comparable yields according to the procedures described for example G1 using 2-(3-methoxy-phenoxy)-acetic acid (commercially available) instead of 3-methyl-sulfanyl-propionic acid in step c).

MS: 390.3 (M−H)⁻

### EXAMPLE G20

### 4-Hydroxy-5-(3-morpholin-4-yl-propyl)-3-(2-m-tolyloxy-acetyl)-5H-furan-2-one

The title compound was obtained in comparable yields according to the procedures described for example G1 using 2-m-tolyloxy-acetic acid (commercially available) instead of 3-methyl-sulfanyl-propionic acid in step c).

MS: 376.4 (M+H)⁺

### EXAMPLE G21

### 4-Hydroxy-3-[2-(2-methoxy-phenoxy)-acetyl]-5-(3-morpholin-4-yl-propyl)-5H-furan-2-one

The title compound was obtained in comparable yields according to the procedures described for example G1 using 2-(2-methoxy-phenoxy)-acetic acid (commercially available) instead of 3-methyl-sulfanyl-propionic acid in step c).

MS: 392.2 (M+H)⁺

### EXAMPLE G22

### 3-[2-(2,3-Dimethyl-phenoxy)-acetyl]-4-hydroxy-5-(3-morpholin-4-yl-propyl)-5H-furan-2-one

The title compound was obtained in comparable yields according to the procedures described for example G1 using 2-(2,3-Dimethyl-phenoxy)-acetic acid (commercially available) instead of 3-methyl-sulfanyl-propionic acid in step c).

MS: 390.3 (M+H)⁺

### EXAMPLE G23

### 4-Hydroxy-5-(3-morpholin-4-yl-propyl)-3-(4-phenyl-butyryl)-5H-furan-2-one

The title compound was obtained in comparable yields according to the procedures described for example G1 using 4-phenyl-butyric acid (commercially available) instead of 3-methyl-sulfanyl-propionic acid in step c).

MS: 372.2 (M−H)⁻

### EXAMPLE G24

### 4-Hydroxy-5-(3-morpholin-4-yl-propyl)-3-(2-naphthalen-2-yl-acetyl)-5H-furan-2-one

The title compound was obtained in comparable yields according to the procedures described for example G1 using 2-naphthalen-2-yl-acetic acid (commercially available) instead of 3-methyl-sulfanyl-propionic acid in step c).

MS: 396.3 (M+H)⁺

### EXAMPLE G25

### 4-Hydroxy-5-(3-morpholin-4-yl-propyl)-3-[2-(naphthalen-1-yloxy)-acetyl]-5H-furan-2-one

The title compound was obtained in comparable yields according to the procedures described for example G1 using 2-(naphthalen-1-yloxy)-acetic acid (commercially available) instead of 3-methyl-sulfanyl-propionic acid in step c).

MS: 410.3 (M−H)⁻

### EXAMPLE G26

### 4-Hydroxy-3-(2-1H-indol-3-yl-acetyl)-5-(3-morpholin-4-yl-propyl)-5H-furan-2-one

The title compound was obtained in comparable yields according to the procedures described for example G1 using 2-1H-indol-3-yl-acetic acid instead of 3-methyl-sulfanyl-propionic acid in step c).

MS: 385.3 (M+H)⁺

### EXAMPLE G27

### 4-Hydroxy-3-(3-1H-indol-3-yl-propionyl)-5-(3-morpholin-4-yl-propyl)-5H-furan-2-one

The title compound was obtained in comparable yields according to the procedures described for example G1 using 3-1H-indol-3-yl-propionic acid (commercially available) instead of 3-methyl-sulfanyl-propionic acid in step c).

MS: 399.4 (M+H)⁺

### EXAMPLE G28

### 3-[2-(2-Acetyl-1,2-dihydro-isoquinolin-1-yl)-acetyl]-4-hydroxy-5-(3-morpholin-4-yl-propyl)-5H-furan-2-one

The title compound was obtained in comparable yields according to the procedures described for example G1 using 2-(2-acetyl-1,2-dihydro-isoquinolin-1-yl)-acetic acid (commercially available) instead of 3-methyl-sulfanyl-propionic acid in step c).

MS: 414.4 (M+H)⁺

### EXAMPLE G29

### 3-(3,3-Diphenyl-propionyl)-4-hydroxy-5-(3-morpholin-4-yl-propyl)-5H-furan-2-one

The title compound was obtained in comparable yields according to the procedures described for example G1 using 3,3-diphenyl-propionic acid (commercially available) instead of 3-methyl-sulfanyl-propionic acid in step c).

MS: 436.4 (M+H)⁺

### EXAMPLE G30

### 4-Hydroxy-5-(3-morpholin-4-yl-propyl)-3-(2-9H-thioxanthen-9-yl-acetyl)-5H-furan-2-one

The title compound was obtained in comparable yields according to the procedures described for example G1 using 2-9H-thioxanthen-9-yl-acetic acid (prepared according to Jilek, Jiri O.; Holubek, Jiri; Svatek, Emil; Ryska, Miroslav; Pomykacek, Josef; Protiva, Miroslav. Collection of Czechoslovak Chemical Communications (1979), 44(7), 2124-2138) instead of 3-methyl-sulfanyl-propionic acid in step c).

MS: 466.3 (M+H)⁺

### EXAMPLE H1

### 5-[2-(4-Benzyloxy-phenyl)-ethyl]-3-(4-cyclohexyl-butyryl)-4-hydroxy-5H-furan-2-one

### a) 5-[2-(4-Benzyloxy-phenyl)ethyl]-4-methoxy-5H-furan-2-one

To a solution of 20 ml of LDA (2M in THF) and 130 ml of THF was added at −95° C. to −100° C. a solution of 5.47 g of 3(E)-methoxy-acrylic acid methyl ester in 4.5 ml of THF within 1 min, stirring was continued at the same temperature for 5 min, which was followed by the addition of a pre-cooled (−78° C.) solution of 33 mmole of the 3-(4-benzyloxy-phenyl)-propionaldehyde in 4.5 ml of THF within 2 min and stirring was continued at −100° C. for 30 min and at −78° C. for 1 h. The cold solution was poured onto 130 ml of ice-water, the pH was adjusted to 4 with 6.5 ml of aqueous HCl (37%) and the layers were separated. The aqueous layer was extracted twice with dichloromethane, the organic layers were washed with brine, dried and evaporated. The residue was chromatographed on silica (n-heptane/AcOEt, various ratios) to give the 5-[2-(4-benzyloxy-phenyl)ethyl]-4-methoxy-5H-furan-2-one in 30-40% yield.

MS: 325.2 (M+H)⁺

### b) 5-[2-(4-Benzyloxy-phenyl)-ethyl]-4-hydroxy-5H-furan-2-one

A mixture of the 5-[2-(4-benzyloxy-phenyl)ethyl]-4-methoxy-5H-furan-2-one (10 mmole) and 15 ml of aqueous HCl (37%) was stirred at 40° C. until completion of the reaction. The suspension was filtered and the residue washed with ice-cold water and dried. An oily reaction mixture was extracted with dichloromethane, the organic layers were washed with brine, dried and evaporated. The residue was either triturated with AcOEt/hexane or chromatographed with dichloromethane/MeOH (various ratios) to give 5-[2-(4-benzyloxy-phenyl)-ethyl]-4-hydroxy-5H-furan-2-one in 60-90% yield.

MS: 310.2 (M)⁺

### c) 5-[2-(4-Benzyloxy-phenyl)-ethyl]-3-(4-cyclohexyl-butyryl)-4-hydroxy-5H-furan-2-one

To a suspension of the 5-[2-(4-benzyloxy-phenyl)-ethyl]-4-hydroxy-5H-furan-2-one (0.2 mmole), NEt₃ (0.68 mmole), DMAP (0.066 mmole) and EDC (0.44 mmole) in 2 ml of THF was added at 22° C. 4-cyclohexyl-butyric acid (0.22 mmole) (commercial available) and stirring was continued until completion of the reaction. The pH of the reaction mixture was adjusted to 3 using aqueous HCl (2 N), the aqueous solution was saturated with NaCl, the organic layer was separated, washed with brine dried and evaporated. The residue was purified on preparative HPLC (RP-18, CH₃CN/H₂O, gradient) to give the 5-[2-(4-benzyloxy-phenyl)-ethyl]-3-(4-cyclohexyl-butyryl)-4-hydroxy-5H-furan-2-one in 10-60% yield.

MS: 463.2 (M+H)⁺

### EXAMPLE 11

### 3-Cyclohexanecarbonyl-4-hydroxy-5-methyl-5-phenethyl-5H-furan-2-one

### a) 4-Methoxy-5-methyl-5-phenethyl-5H-furan-2-one

To a solution of 20 ml of LDA (2M in THF) and 130 ml of THF was added at −95° C. to −100° C. a solution of 5.47 g of 3(E)-methoxy-acrylic acid methyl ester in 4.5 ml of THF within 1 min, stirring was continued at the same temperature for 5 min, which was followed by the addition of a pre-cooled (−78° C.) solution of 33 mmole of the 4-phenyl-butan-2-one in 4.5 ml of THF within 2 min and stirring was continued at −100° C. for 30 min and at −78° C. for 1 h. The cold solution was poured onto 130 ml of ice-water, the pH was adjusted to 4 with 6.5 ml of aqueous HCl (37%) and the layers were separated. The aqueous layer was extracted twice with dichloromethane, the organic layers were washed with brine, dried and evaporated. The residue was chromatographed on silica (n-heptane/AcOEt, various ratios) to give 4-methoxy-5-methyl-5-phenethyl-5H-furan-2-one in 30-40% yield.

MS: 233.2 (M+H)+

### b) 4-Hydroxy-5-methyl-5-phenethyl-5H-furan-2-one

A mixture of the 4-methoxy-5-methyl-5-phenethyl-5H-furan-2-one (10 mmole) and 15 ml of aqueous HCl (37%) was stirred at 40° C. until completion of the reaction. The suspension was filtered and the residue washed with ice-cold water and dried. An oily reaction mixture was extracted with dichloromethane, the organic layers were washed with brine, dried and evaporated. The residue was either triturated with AcOEt/hexane or chromatographed with dichloromethane/MeOH (various ratios) to give 4-hydroxy-5-methyl-5-phenethyl-5H-furan-2-one in 60-90% yield.

MS: 218.2 (M)⁺

### c) 3-Cyclohexanecarbonyl-4-hydroxy-5-methyl-5-phenethyl-5H-furan-2-one

To as suspension of the 4-hydroxy-5-methyl-5-phenethyl-5H-furan-2-one (0.2 mmole), NEt₃ (0.68 mmole), DMAP (0.066 mmole) and EDC (0.44 mmole) in 2 ml of THF was added at 22° C. cyclohexanecarboxylic acid (0.22 mmole) (commercially available) and stirring was continued until completion of the reaction. The pH of the reaction mixture was adjusted to 3 using aqueous HCl (2 N), the aqueous solution was saturated with NaCl, the organic layer was separated, washed with brine dried and evaporated. The residue was purified on preparative HPLC (RP-18, CH₃CN/H₂O, gradient) to give the 3-cyclohexanecarbonyl-4-hydroxy-5-methyl-5-phenethyl-5H-furan-2-one in 10-60% yield.

MS: 327.2 (M−H)⁻

### EXAMPLE 12

### 3-(4-Cyclohexyl-butyryl)-4-hydroxy-5-methyl-5-phenethyl-5H-furan-2-one

The title compound was obtained in comparable yields according to the procedures described for example F1 using 4-cyclohexyl-butyric acid (commercially available) instead of cyclohexanecarboxylic acid in step c).

MS: 369.2 (M−H)⁻

### EXAMPLE 13

### 3-[3-(4-tert-Butyl-phenyl)-2-methyl-propionyl]-4-hydroxy-5-methyl-5-phenethyl-5H-furan-2-one

The title compound was obtained in comparable yields according to the procedures described for example F1 using 3-(4-tert-Butyl-phenyl)-2-methyl-propionic acid (prepared according to Kuchar, Miroslav; Rejholec, Vaclav; Roubal, Zdenek; Nemecek, Oldrich; Collect. Czech. Chem. Commun. (1979), 44(1), 183-93) instead of cyclohexanecarboxylic acid in step c).

MS: 419.2 (M−H)⁻

### EXAMPLE 14

### 4-Hydroxy-3-[(2-methoxy-phenoxy)-acetyl]-5-methyl-5-phenethyl-5H-furan-2-one

The title compound was obtained in comparable yields according to the procedures described for example F1 using (2-methoxy-phenoxy)-acetic acid (commercially available) instead of cyclohexanecarboxylic acid in step c).

MS: 381.2 (M−H)⁻

### EXAMPLE 15

### 4-Hydroxy-3-[(1H-indol-3-yl)-acetyl]-5-methyl-5-phenethyl-5H-furan-2-one

The title compound was obtained in comparable yields according to the procedures described for example F1 using (1H-indol-3-yl)-acetic acid (commercially available) instead of cyclohexanecarboxylic acid in step c).

MS: 374.2 (M−H)⁻

### EXAMPLE 16

### 3-(3,3-Diphenyl-propionyl)-4-hydroxy-5-methyl-5-phenethyl-5H-furan-2-one

The title compound was obtained in comparable yields according to the procedures described for example F1 using 3,3-diphenyl-propionic acid (commercially available) instead of cyclohexanecarboxylic acid in step c).

MS: 425.3 (M−H)⁻

### EXAMPLE 17

### 3-[(9H-Fluoren-9-yl)-acetyl]-4-hydroxy-5-methyl-5-phenethyl-5H-furan-2-one

The title compound was obtained in comparable yields according to the procedures described for example F1 using (9H-fluoren-9-yl)-acetic acid (commercially available) instead of cyclohexanecarboxylic acid in step c).

MS: 423.2 (M−H)⁻

### EXAMPLE J1

### 3-Cyclohexanecarbonyl-4-hydroxy-5-phenethyl-5-phenyl-5H-furan-2-one

### a) 4-Methoxy-5-phenethyl-5-phenyl-5H-furan-2-one

To a solution of 20 ml of LDA (2M in THF) and 130 ml of THF was added at −95° C. to −100° C. a solution of 5.47 g of 3(E)-methoxy-acrylic acid methyl ester in 4.5 ml of THF within 1 min, stirring was continued at the same temperature for 5 min, which was followed by the addition of a pre-cooled (−78° C.) solution of 33 mmole of the 1,3-diphenyl-propan-1-one in 4.5 ml of THF within 2 min and stirring was continued at −100° C. for 30 min and at −78° C. for 1 h. The cold solution was poured onto 130 ml of ice-water, the pH was adjusted to 4 with 6.5 ml of aqueous HCl (37%) and the layers were separated. The aqueous layer was extracted twice with dichloromethane, the organic layers were washed with brine, dried and evaporated. The residue was chromatographed on silica (n-heptane/AcOEt, various ratios) to give 4-methoxy-5-phenethyl-5-phenyl-5H-furan-2-one in 30-40% yield.

MS: 294.2 (M)⁺

### b) 4-Hydroxy-5-phenethyl-5-phenyl-5H-furan-2-one

A mixture of the 4-methoxy-5-phenethyl-5-phenyl-5H-furan-2-one (10 mmole) and 15 ml of aqueous HCl (37%) was stirred at 40° C. until completion of the reaction. The suspension was filtered and the residue washed with ice-cold water and dried. An oily reaction mixture was extracted with dichloromethane, the organic layers were washed with brine, dried and evaporated. The residue was either triturated with AcOEt/hexane or chromatographed with dichloromethane/MeOH (various ratios) to give 4-hydroxy-5-phenethyl-5-phenyl-5H-furan-2-one in 60-90% yield.

MS: 176.0 (M−C₈H₈)⁺

### c) 3-Cyclohexanecarbonyl-4-hydroxy-5-phenethyl-5-phenyl-5H-furan-2-one

To as suspension of the 4-hydroxy-5-phenethyl-5-phenyl-5H-furan-2-one (0.2 mmole), NEt₃ (0.68 mmole), DMAP (0.066 mmole) and EDC (0.44 mmole) in 2 ml of THF was added at 22° C. cyclohexanecarboxylic acid (0.22 mmole) (commercially available) and stirring was continued until completion of the reaction. The pH of the reaction mixture was adjusted to 3 using aqueous HCl (2 N), the aqueous solution was saturated with NaCl, the organic layer was separated, washed with brine dried and evaporated. The residue was purified on preparative HPLC (RP-18, CH₃CN/H₂O, gradient) to give the 3-cyclohexanecarbonyl-4-hydroxy-5-phenethyl-5-phenyl-5H-furan-2-one in 10-60% yield.

MS: 389.1 (M−H)⁻

### EXAMPLE J2

### 4-Hydroxy-3-[(2-methoxy-phenoxy)-acetyl]-5-phenethyl-5-phenyl-5H-furan-2-one

The title compound was obtained in comparable yields according to the procedures described for example J1 using (2-methoxy-phenoxy)-acetic acid (commercially available) instead of cyclohexanecarboxylic acid in step c).

MS: 443.1 (M−H)⁻

### EXAMPLE J3

### 4-Hydroxy-3-[(H-indol-3-yl)-acetyl]-5-phenethyl-5-phenyl-5H-furan-2-one

The title was obtained in comparable yields according to the procedures described for example J1 using (1H-indol-3-yl)-acetic acid (commercially available) instead of cyclohexanecarboxylic acid in step c).

MS: 436.1 (M−H)⁻

### EXAMPLE J4

### 3-(3,3-Diphenyl-propionyl)-4-hydroxy-5-phenethyl-5-phenyl-5H-furan-2-one

The title compound was obtained in comparable yields according to the procedures described for example J1 using 3,3-diphenyl-propionic acid (commercially available) instead of cyclohexanecarboxylic acid in step c).

MS: 384.2 (M−C₈H₈)⁺

### EXAMPLE J5

### 3-[(9H-Fluoren-9-yl)-acetyl]-4-hydroxy-5-phenethyl-5-phenyl-5H-furan-2-one

The title compound was obtained in comparable yields according to the procedures described for example F1 using (9H-Fluoren-9-yl)-acetic acid (commercially available) instead of cyclohexanecarboxylic acid in step c).

MS: 485.2 (M−H)⁻

### EXAMPLE K1

### 4-Hydroxy-3-(3-methylsulfanyl-propionyl)-5-phenethyl-1,5-dihydro-pyrrol-2-one

### a) Rac-{1-[(2,2-dimethyl-4,6-dioxo-[1,3]dioxan-5-ylidene)-hydroxy-methyl]-3-phenyl-propyl}-carbamic acid tert-butyl ester

To a solution of 4.00 g of rac-homophenylalanine in 80 ml of dichloromethane was subsequently added at 22° C. 2.17 g of Meldrum's acid and 4.02 g of DMAP followed by a solution of 3.16 g of DCC in 20 ml of dichloromethane over 5 min and stirring was continued for 16 h. The suspension was filtered, the filtrate washed with aqueous HCl and water, dried and evaporated. The residue was triturated with 60 ml of methanol over 15 min, the suspension was diluted with 60 ml of diethylether, filtered and the residue was washed with MeOH/diethylether (1:1, 20 ml) and dried to give 3.54 g of rac-{1-[(2,2-dimethyl-4,6-dioxo-[1,3]dioxan-5-ylidene)-hydroxy-methyl]-3-phenyl-propyl}-carbamic acid tert-butyl ester as a white solid.

MS: 423.2 (M+NH₄)⁺.

### b) Rac-3-hydroxy-5-oxo-2-phenethyl-2,5-dihydro-pyrrole-1-carboxylic acid tert-butyl ester

A suspension of 3.40 g of rac-{1-[(2,2-dimethyl-4,6-dioxo-[1,3]dioxan-5-ylidene)-hydroxy-methyl]-3-phenyl-propyl}-carbamic acid tert-butyl ester and 40 ml of methanol was heated to reflux temperature for 1 h and evaporated to give 2.53 g of rac-3-hydroxy-5-oxo-2-phenethyl-2,5-dihydro-pyrrole-1-carboxylic acid tert-butyl ester as a colourless foam.

MS: 304.1 (M+H)⁺

### c) Rac-4-hydroxy-5-phenethyl-1,5-dihydro-pyrrol-2-one

A solution of 1.58 g of rac-3-hydroxy-5-oxo-2-phenethyl-2,5-dihydro-pyrrole-1-carboxylic acid tert-butyl ester in 32 ml of dichloromethane was treated at 22° C. with 2.0 ml of trifluoroacetic acid and stirring was continued for 16 h. The solution was evaporated to dryness, the residue dissolved in 8 ml of diethylether and stirring was continued until the crystallization set in. The suspension was diluted with 8 ml of n-heptane, stirred for 15 min and filtered. The residue was washed with n-heptane and dried to give 0.85 g of rac-4-hydroxy-5-phenethyl-1,5-dihydro-pyrrol-2-one as a white solid.

MS: 204.2 (M+H)⁺

### d) 4-Hydroxy-3-(3-methylsulfanyl-propionyl)-5-phenethyl-1,5-dihydro-pyrrol-2-one

To as suspension of the rac-4-hydroxy-5-phenethyl-1,5-dihydro-pyrrol-2-one (0.2 mmole), NEt₃ (0.68 mmole), DMAP (0.066 mmole) and EDC (0.44 mmole) in 2 ml of THF was added at 22° C. 3-methylsulfanyl-propionic acid (0.22 mmole) (commercially available) and stirring was continued until completion of the reaction. The pH of the reaction mixture was adjusted to 3 using aqueous HCl (2 N), the aqueous solution was saturated with NaCl, the organic layer was separated, washed with brine dried and evaporated. The residue was purified on preparative HPLC (RP-18, CH₃CN/H₂O, gradient) to give the 4-hydroxy-3-(3-methylsulfanyl-propionyl)-5-phenethyl-1,5-dihydro-pyrrol-2-one in 20-60% yield.

MS: 304.1 (M−H)⁻

### EXAMPLE K2

### 3-Cyclopropanecarbonyl-4-hydroxy-5-phenethyl-1,5-dihydro-pyrrol-2-one

The title compound was obtained in comparable yields according to the procedures described for example K1 using cyclopropanecarboxylic acid (commercially available) instead of 3-methylsulfanyl-propionic acid in step d).

MS: 270.3 (M−H)⁻

### EXAMPLE K3

### 4-Hydroxy-3-(1-methyl-cyclopropanecarbonyl)-5-phenethyl-1,5-dihydro-pyrrol-2-one

The title compound was obtained in comparable yields according to the procedures described for example K1 using 1-methyl-cyclopropanecarboxylic acid (commercially available) instead of 3-methylsulfanyl-propionic acid in step d).

MS: 283.3 (M−H)⁻

### EXAMPLE K4

### 4-Hydroxy-5-phenethyl-3-(tetrahydro-furan-2-carbonyl)-1,5-dihydro-pyrrol-2-one

The title compound was obtained in comparable yields according to the procedures described for example K1 using tetrahydro-furan-2-carboxylic acid (commercially available) instead of 3-methylsulfanyl-propionic acid in step d).

MS: 302.2 (M+H)⁺

### EXAMPLE K5

### 3-(4-Cyclohexyl-butyryl)-4-hydroxy-5-phenethyl-1,5-dihydro-pyrrol-2-one

The title compound was obtained in comparable yields according to the procedures described for example K1 using 4-cyclohexyl-butyric acid (commercially available) instead of 3-methylsulfanyl-propionic acid in step d).

MS: 356.2 (M+H)⁺

### EXAMPLE K6

### 4-Hydroxy-5-phenethyl-3-(thieno[2,3-c]pyridine-7-carbonyl)-1,5-dihydro-pyrrol-2-one

The title compound was obtained in comparable yields according to the procedures described for example K1 using thieno[2,3-c]pyridine-7-carboxylic acid (prepared according to Bass, R. J.; Popp, F. D.; Kant, J. Journal of Heterocyclic Chemistry (1984), 21(4), 1119-20) instead of 3-methylsulfanyl-propionic acid in step d).

MS: 365.1 (M+H)⁺

### EXAMPLE K7

### 4-Hydroxy-3-(5-methyl-pyrazine-2-carbonyl)-5-phenethyl-1,5-dihydro-pyrrol-2-one

The title compound was obtained in comparable yields according to the procedures described for example K1 using 5-methyl-pyrazine-2-carboxylic acid (commercially available) instead of 3-methylsulfanyl-propionic acid in step d).

MS: 324.1 (M+H)⁺

### EXAMPLE K8

### 4-Hydroxy-3-(isoquinoline-3-carbonyl)-5-phenethyl-1,5-dihydro-pyrrol-2-one

The title compound was obtained in comparable yields according to the procedures described for example K1 using isoquinoline-3-carboxylic acid (commercially available) instead of 3-methylsulfanyl-propionic acid in step d).

MS: 358.1 (M+H)⁺

### EXAMPLE K9

### 3-(Benzo[1,2,3]thiadiazole-5-carbonyl)-4-hydroxy-5-phenethyl-1,5-dihydro-pyrrol-2-one

The title compound was obtained in comparable yields according to the procedures described for example K1 using benzo[1,2,3]thiadiazole-5-carboxylic acid (commercially available) instead of 3-methylsulfanyl-propionic acid in step d).

MS: 364.1 (M−H)⁻

### EXAMPLE K10

### 4-Hydroxy-3-(3-methyl-furan-2-carbonyl)-5-phenethyl-1,5-dihydro-pyrrol-2-one

The title compound was obtained in comparable yields according to the procedures described for example K1 using 3-methyl-furan-2-carboxylic acid (commercially available) instead of 3-methylsulfanyl-propionic acid in step d).

MS: 319.2 (M−H)⁻

### EXAMPLE K11

### 3-(2,3-Dihydro-benzofuran-7-carbonyl)-4-hydroxy-5-phenethyl-1,5-dihydro-pyrrol-2-one

The title compound was obtained in comparable yields according to the procedures described for example K1 using 2,3-dihydro-benzofuran-7-carboxylic acid (prepared according to Voelter, Wolfgang; El-Abadelah, Mustafa M.; Sabri, Salim S.; Khanfar, Monther A. Zeitschrift fuer Naturforschung, B: Chemical Sciences (1999), 54(11), 1469-1473) instead of 3-methylsulfanyl-propionic acid in step d).

MS: 348.2 (M−H)⁻

### EXAMPLE K12

### 4-Hydroxy-5-phenethyl-3-(1,2,5-trimethyl-1H-pyrrole-3-carbonyl)-1,5-dihydro-pyrrol-2-one

The title compound was obtained in comparable yields according to the procedures described for example K1 using 1,2,5-trimethyl-1H-pyrrole-3-carboxylic acid (commercially available) instead of 3-methylsulfanyl-propionic acid in step d).

MS: 337.2 (M−H)⁻

### EXAMPLE K13

### 4-Hydroxy-5-phenethyl-3-phenylacetyl-1,5-dihydro-pyrrol-2-one

The title compound was obtained in comparable yields according to the procedures described for example K1 using phenyl-acetic acid (commercially available) instead of 3-methylsulfanyl-propionic acid in step d).

MS: 320.1 (M−H)⁻

### EXAMPLE K14

### 4-Hydroxy-3-(2-naphthalen-2-yl-acetyl)-5-phenethyl-1,5-dihydro-pyrrol-2-one

The title compound was obtained in comparable yields according to the procedures described for example K1 using 2-naphthalen-2-yl-acetic acid (commercially available) instead of 3-methylsulfanyl-propionic acid in step d).

MS: 370.2 (M−H)⁻

### EXAMPLE K15

### 4-Hydroxy-3-[2-(3-oxo-indan-1-yl)-acetyl]-5-phenethyl-1,5-dihydro-pyrrol-2-one

The title compound was obtained in comparable yields according to the procedures described for example K1 using 2-(3-oxo-indan-1-yl)-acetic acid (prepared according to Thompson, Hugh W.; Brunskull, Andrew P. J.; Lalancette, Roger A. Acta Crystallographica, Section C: Crystal Structure Communications (1998), C54(6), 829-831) instead of 3-methylsulfanyl-propionic acid in step d).

MS: 374.2 (M−H)⁻

### EXAMPLE K16

### 1-[2-(4-Hydroxy-2-oxo-5-phenethyl-2,5-dihydro-1H-pyrrol-3-yl)-2-oxo-ethyl]-5-methyl-1H-pyrimidine-2,4-dione

The title compound was obtained in comparable yields according to the procedures described for example K1 using

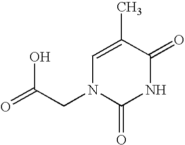

(commercially available) instead of 3-methylsulfanyl-propionic acid in step d).

MS: 368.1 (M−H)⁻

### EXAMPLE K17

### 4-Hydroxy-5-phenethyl-3-(2-phenyl-propionyl)-1,5-dihydro-pyrrol-2-one

The title compound was obtained in comparable yields according to the procedures described for example K1 using 2-phenyl-propionic acid (commercially available) instead of 3-methylsulfanyl-propionic acid in step d).

MS: 336.2 (M+H)⁺

### EXAMPLE K18

### 4-Hydroxy-3-[2-(6-methoxy-naphthalen-2-yl)-propionyl]-5-phenethyl-1,5-dihydro-pyrrol-2-one

The title compound was obtained in comparable yields according to the procedures described for example K1 using 2-(6-methoxy-naphthalen-2-yl)-propionic acid (commercially available) instead of 3-methylsulfanyl-propionic acid in step d).

MS: 414.2 (M−H)⁻

### EXAMPLE K19

### 4-Hydroxy-5-phenethyl-3-(3-m-tolyl-propionyl)-1,5-dihydro-pyrrol-2-one

The title compound was obtained in comparable yields according to the procedures described for example K1 using 3-m-tolyl-propionic acid (commercially available) instead of 3-methylsulfanyl-propionic acid in step d).

MS: 348.2 (M−H)⁻

### EXAMPLE K20

### 4-Hydroxy-3-[3-(3-methoxy-phenyl)-propionyl]-5-phenethyl-1,5-dihydro-pyrrol-2-one

The title compound was obtained in comparable yields according to the procedures described for example K1 using 3-(3-methoxy-phenyl)-propionic acid (commercially available) instead of 3-methylsulfanyl-propionic acid in step d).

MS: 364.2 (M−H)⁻

### EXAMPLE K21

### 4-Hydroxy-3-[3-(2-methoxy-phenyl)-propionyl]-5-phenethyl-1,5-dihydro-pyrrol-2-one

The title compound was obtained in comparable yields according to the procedures described for example K1 using 3-(2-methoxy-phenyl)-propionic acid (commercially available) instead of 3-methylsulfanyl-propionic acid in step d).

MS: 364.2 (M−H)⁻

### EXAMPLE K20

### 4-Hydroxy-3-[3-(4-methoxy-phenyl)-propionyl]-5-phenethyl-1,5-dihydro-pyrrol-2-one

The title compound was obtained in comparable yields according to the procedures described for example K1 using 3-(4-methoxy-phenyl)-propionic acid (commercially available) instead of 3-methylsulfanyl-propionic acid in step d).

MS: 364.2 (M−H)⁻

### EXAMPLE K23

### 3-[3-(4-tert-Butyl-phenyl)-2-methyl-propionyl]-4-hydroxy-5-phenethyl-1,5-dihydro-pyrrol-2-one

The title compound was obtained in comparable yields according to the procedures described for example K1 using 3-(4-tert-butyl-phenyl)-2-methyl-propionic acid (prepared according to Kuchar, Miroslav; Rejholec, Vaclav; Roubal, Zdenek; Nemecek, Oldrich; Collect. Czech. Chem. Commun. (1979), 44(1), 183-193) instead of 3-methylsulfanyl-propionic acid in step d).

MS: 406.4 (M+H)⁺

### EXAMPLE K24

### 4-Hydroxy-3-[(2-methoxy-phenoxy)-acetyl]-5-phenethyl-1,5-dihydro-pyrrol-2-one

The title compound was obtained in comparable yields according to the procedures described for example K1 using (2-methoxy-phenoxy)-acetic acid (commercially available) instead of 3-methylsulfanyl-propionic acid in step d).

MS: 368.2 (M+H)⁺

### EXAMPLE K25

### 4-Hydroxy-5-phenethyl-3-(4-phenyl-butyryl)-1,5-dihydro-pyrrol-2-one

The title compound was obtained in comparable yields according to the procedures described for example K1 using 4-phenyl-butyric acid (commercially available) instead of 3-methylsulfanyl-propionic acid in step d).

MS: 348.2 (M−H)⁻

### EXAMPLE K26

### 3-[4-(3,4-Dimethoxy-phenyl)-butyryl]-4-hydroxy-5-phenethyl-1,5-dihydro-pyrrol-2-one

The title compound was obtained in comparable yields according to the procedures described for example K1 using 4-(3,4-dimethoxy-phenyl)-butyric acid (commercially available) instead of 3-methylsulfanyl-propionic acid in step d).

MS: 408.3 (M−H)⁻

### EXAMPLE K27

### N-[2-(4-Hydroxy-2-oxo-5-phenethyl-2,5-dihydro-1H-pyrrol-3-yl)-2-oxo-ethyl]-acetamide

The title compound was obtained in comparable yields according to the procedures described for example K1 using

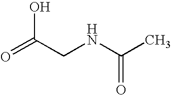

(commercially available) instead of 3-methylsulfanyl-propionic acid in step d).

MS: 301.1 (M−H)⁻

### EXAMPLE K28

### N-[1-(4-Hydroxy-2-oxo-5-phenethyl-2,5-dihydro-1H-pyrrole-3-carbonyl)-3-methylsulfanyl-propyl]-acetamide

The title compound was obtained in comparable yields according to the procedures described for example K1 using

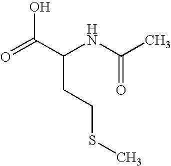

(commercially available) instead of 3-methylsulfanyl-propionic acid in step d).

MS: 377.2 (M+H)⁺

### EXAMPLE K29

### N-[2-(4-Hydroxy-2-oxo-5-phenethyl-2,5-dihydro-1H-pyrrol-3-yl)-2-oxo-ethyl]-N-methyl-benzamide

The title compound was obtained in comparable yields according to the procedures described for example K1 using

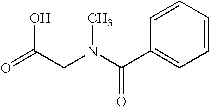

(commercially available) instead of 3-methylsulfanyl-propionic acid in step d).

MS: 379.2 (M+H)⁺

### EXAMPLE K30

### N-[2-(4-Hydroxy-2-oxo-5-phenethyl-2,5-dihydro-1H-pyrrol-3-yl)-2-oxo-ethyl]-4-methyl-benzamide

The title compound was obtained in comparable yields according to the procedures described for example K1 using

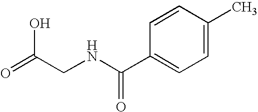

(commercially available) instead of 3-methylsulfanyl-propionic acid in step d).

MS: 379.2 (M+H)⁺

### EXAMPLE K31

### N-[2-(4-Hydroxy-2-oxo-5-phenethyl-2,5-dihydro-1H-pyrrol-3-yl)-2-oxo-ethyl]-nicotinamide

The title compound was obtained in comparable yields according to the procedures described for example K1 using

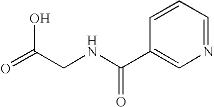

(commercially available) instead of 3-methylsulfanyl-propionic acid in step d).

MS: 364.2 (M−H)⁻

### EXAMPLE K32

### [2-(4-Hydroxy-2-oxo-5-phenethyl-2,5-dihydro-1H-pyrrol-3-yl)-1-methyl-2-oxo-ethyl]-carbamic acid tert-butyl ester

The title compound was obtained in comparable yields according to the procedures described for example K1 using

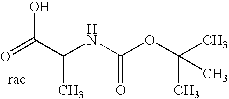

(commercially available) instead of 3-methylsulfanyl-propionic acid in step d).

MS: 375.3 (M+H)⁺

### EXAMPLE K33

### [1-Benzyl-2-(4-hydroxy-2-oxo-5-phenethyl-2,5-dihydro-1H-pyrrol-3-yl)-2-oxo-ethyl]-carbamic acid tert-butyl ester

The title compound was obtained in comparable yields according to the procedures described for example K1 using

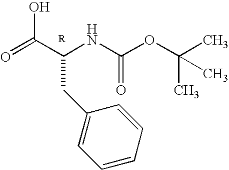

(commercially available) instead of 3-methylsulfanyl-propionic acid in step d).

MS: 451.2 (M+H)⁺

### EXAMPLE K34

### 2-(4-Hydroxy-2-oxo-5-phenethyl-2,5-dihydro-1H-pyrrole-3-carbonyl)-pyrrolidine-1-carboxylic acid tert-butyl ester

The title compound was obtained in comparable yields according to the procedures described for example K1 using

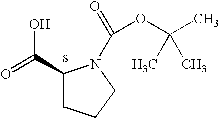

(commercially available) instead of 3-methylsulfanyl-propionic acid in step d).

MS: 401.4 (M+H)⁺

### EXAMPLE K35

### 2-(4-Hydroxy-2-oxo-5-phenethyl-2,5-dihydro-1H-pyrrole-3-carbonyl)-piperidine-1-carboxylic acid tert-butyl ester

The title compound was obtained in comparable yields according to the procedures described for example K1 using

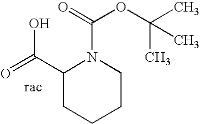

(commercially available) instead of 3-methylsulfanyl-propionic acid in step d).

MS: 415.3 (M+H)⁺

### EXAMPLE K36

### 3-(4-Hydroxy-2-oxo-5-phenethyl-2,5-dihydro-1H-pyrrole-3-carbonyl)-3,4-dihydro-1H-isoquinoline-2-carboxylic acid tert-butyl ester

The title compound was obtained in comparable yields according to the procedures described for example K1 using

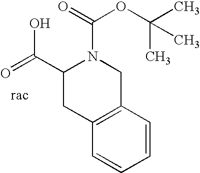

(commercially available) instead of 3-methylsulfanyl-propionic acid in step d).

MS: 463.3 (M+H)⁺

### EXAMPLE K37

### [1-(4-Benzyloxy-benzyl)-2-(4-hydroxy-2-oxo-5-phenethyl-2,5-dihydro-1H-pyrrol-3-yl)-2-oxo-ethyl]-carbamic acid tert-butyl ester

The title compound was obtained in comparable yields according to the procedures described for example K1 using

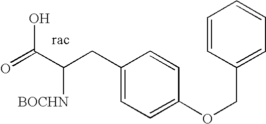

(commercially available) instead of 3-methylsulfanyl-propionic acid in step d).

MS: 574.3 (M+NH₄)⁺

### EXAMPLE K38

### 3-[2-Amino-3-(4-benzyloxy-phenyl)-propionyl]-4-hydroxy-5-phenethyl-1,5-dihydro-pyrrol-2-one; compound with trifluoro-acetic acid

The title compound was prepared from the corresponding BOC-protected precursor (Example K37) by deprotection using CF₃COOH.

MS: 457.2 (M+H)⁺

### EXAMPLE K39

### 4-Hydroxy-3-[(H-indol-3-yl)-acetyl]-5-phenethyl-1,5-dihydro-pyrrol-2-one

The title compound was obtained in comparable yields according to the procedures described for example K1 using -[(1H-indol-3-yl)-acetic acid (commercially available) instead of 3-methylsulfanyl-propionic acid in step d).

MS: 361.1 (M+H)⁺

### EXAMPLE K40

### 3-{[1-(4-Fluoro-benzyl)-1H-indol-3-yl]-acetyl}-4-hydroxy-5-phenethyl-1,5-dihydro-pyrrol-2-one

The title compound was obtained in comparable yields according to the procedures described for example K1 using 1-(4-Fluoro-benzyl)-1H-indol-3-yl]-acetic acid (commercially available) instead of 3-methylsulfanyl-propionic acid in step d).

MS: 469.2 (M+H)⁺

### EXAMPLE K41

### 4-Hydroxy-3-(indol-1-yl-acetyl)-5-phenethyl-1,5-dihydro-pyrrol-2-one

The title compound was obtained in comparable yields according to the procedures described for example K1 using indol-1-yl-acetic acid (commercially available) instead of 3-methylsulfanyl-propionic acid in step d).

MS: 361.2 (M+H)⁺

### EXAMPLE K42

### 4-Hydroxy-3-(3-1H-indol-3-yl-propionyl)-5-phenethyl-1,5-dihydro-pyrrol-2-one

The title compound was obtained in comparable yields according to the procedures described for example K1 using 3-1H-indol-3-yl-propionic acid (commercially available) instead of 3-methylsulfanyl-propionic acid in step d).

MS: 373.1 (M−H)⁻

### EXAMPLE K43

### 3-(2-Benzo[b]thiophen-3-yl-acetyl)-4-hydroxy-5-phenethyl-1,5-dihydro-pyrrol-2-one

The title compound was obtained in comparable yields according to the procedures described for example K1 using 2-benzo[b]thiophen-3-yl-acetic acid (commercially available) instead of 3-methylsulfanyl-propionic acid in step d).

MS: 378.2 (M+H)⁺

### EXAMPLE K44

### 3-(3,3-Diphenyl-propionyl)-4-hydroxy-5-phenethyl-1,5-dihydro-pyrrol-2-one

The title compound was obtained in comparable yields according to the procedures described for example K1 using 3,3-diphenyl-propionylic acid (commercially available) instead of 3-methylsulfanyl-propionic acid in step d).

MS: 412.2 (M+H)⁺

### EXAMPLE K45

### 3-(2,3-Diphenyl-propionyl)-4-hydroxy-5-phenethyl-1,5-dihydro-pyrrol-2-one

The title compound was obtained in comparable yields according to the procedures described for example K1 using 2,3-Diphenyl-propionic acid (commercially available) instead of 3-methylsulfanyl-propionic acid in step d).

MS: 412.3 (M+H)⁺

### EXAMPLE K46

### 3-(Carbazol-9-yl-acetyl)-4-hydroxy-5-phenethyl-1,5-dihydro-pyrrol-2-one

The title compound was obtained in comparable yields according to the procedures described for example K1 using carbazol-9-yl-acetic acid (commercially available) instead of 3-methylsulfanyl-propionic acid in step d).

MS: 411.3 (M+H)⁺

¹H-NMR (300 MHz, internal standard TMS, J values in Hz, d6-DMSO): 9.20 (s, br., 1H), 8.15 (d, J=7.7, 2H), 7.50-7.10 (m, 11H), 5.69 (s, 2H), 4.00 (J=7.6 and 4, 1H), 2.95 (s, br. 1H), 2.80-2.65 (m, 2H), 2.20-2.00 (m 1H), 1.95-1.80 (m, 1H)

1. A method of treating Alzheimer's disease comprising administering to an individual a therapeutically effective amount of a compound of formula I 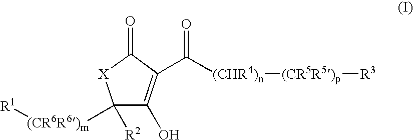
wherein
X is O or NH;
R¹ is lower alkyl, cycloalkyl, heterocycloalkyl or aryl, wherein the aryl ring is unsubstituted or substituted by benzyloxy;
R² is H, lower alkyl or aryl;
R³ is lower alkyl,
—SCH₃,
acetyl, 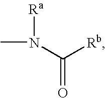
wherein R^{a} is H or lower alkyl, R^{b} is lower alkyl, heteroaryl, —OC(CH₃)₃ or aryl, wherein the aryl ring is unsubstituted or substituted by lower alkyl,
cycloalkyl, wherein the cycloalkyl ring is unsubstituted or substituted by lower alkyl or aryl,
heterocycloalkyl, wherein the heterocycloalkyl ring is unsubstituted or substituted by —COOC(CH₃)₃, or
(CH═CR′)ₒ-aryl, wherein the aryl ring is unsubstituted or substituted by lower alkyl, alkoxy, hydroxyl, benzyloxy, halogen, acetyl, —(CH₂)₂NHSO₂Ph, —NHCO(CH₂)₂NHCOOC(CH₃)₃ or —(CH₂)₂NHCOC₆H₃OCH₃Cl, or for the non aromatic part of fused ring system also by oxo,
o is 0 or 1;
R′ is H,
lower alkyl,
aryloxy, wherein the aryl ring is unsubstituted or substituted by lower alkyl or alkoxy, or
(CH═CH)_{q}-heteroaryl, wherein the heteroaryl ring is unsubstituted or substituted by lower alkyl, acetyl, alkoxy, halogen, —COOC(CH₃)₃ or by halogen substituted benzyl, or for the non aromatic part of fused ring system also by oxo;
q is 0 or 1;
R⁴ is H, lower alkyl, —(CH₂)₂SCH₃, —NHCOCH₃, —NHSO₂p-Cl-Ph, amino, —NHCOOC(CH₃)₃, hydroxyl, aryl, benzyl or halogen substituted benzyl;
R⁵ and R^{5′} are each independently selected from H, lower alkyl or aryl;
R⁶ and R^{6′} are each independently selected from H, lower alkyl or —SCH₃;
m is 1, 2 or 3;
n is 0 or 1; and
p is 0, 1, 2 or 3;
or a pharmaceutically acceptable salt thereof.

2. The method of claim 1, wherein the compound of formula I has the formula Ia 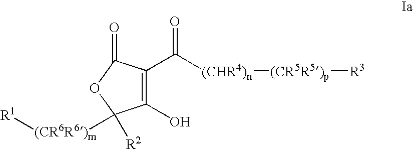
or a pharmaceutically acceptable salt thereof.

3. The method of claim 2, wherein in the compound of formula Ia
R¹ is lower alkyl, cycloalkyl, heterocycloalkyl, or aryl, wherein the aryl ring is unsubstituted or substituted by benzyloxy;
R² is H, lower alkyl or aryl;
R³ is lower alkyl,
—SCH₃,
acetyl,
cycloalkyl, wherein the cycloalkyl ring is unsubstituted or substituted by lower alkyl or aryl,
heterocycloalkyl, or
(CH═CR′)ₒ-aryl, wherein the aryl ring is unsubstituted or substituted by lower alkyl, alkoxy, hydroxyl, benzyloxy, halogen, acetyl, —(CH₂)₂NHSO₂Ph, —NHCO(CH₂)₂NHCOOC(CH₃)₃ or —(CH₂)₂NHCOC₆H₃OCH₃Cl;
o is 0 or 1;
R′ is H,
lower alkyl,
aryloxy, wherein the aryl ring is unsubstituted or substituted by lower alkyl or alkoxy, or
(CH═CH)_{q}-heteroaryl, wherein the heteroaryl ring is unsubstituted or substituted by lower alkyl, acetyl, alkoxy, halogen, or by halogen substituted benzyl;
q is 0 or 1;
R⁴ is H, lower alkyl, —(CH₂)₂SCH₃, —NHSO₂p-Cl-Ph, amino, —NHCOOC(CH₃)₃, hydroxyl, aryl, benzyl or halogen substituted benzyl;
R⁵ and R^{5′} are each independently selected from H, lower alkyl or aryl;
R⁶ and R^{6′} are each independently selected from H, lower alkyl or —SCH₃;
m is 1, 2 or 3;
n is 0 or 1; and
p is 0, 1, 2 or 3;
or a pharmaceutically acceptable salt thereof.

4. The method of claim 3, wherein in the compound of formula Ia
R¹ is methyl, cyclohexyl, phenyl, morpholin-4-yl or 4-benzyloxy-phenyl;
R² is H, methyl or phenyl;
R³ is methyl,
—SCH₃,
acetyl,
cycloalkyl, wherein the cycloalkyl ring is unsubstituted or substituted by methyl, tert-butyl or phenyl,
tetrahydro-furan-2-yl, pyrrolidine-2-yl, 1-tert-butyloxycarbonylpyrrolidine-2-yl, piperidine-2-yl, 1-tert-butyloxycarbonyl piperidine-2-yl, or
(CH═CR′)ₒ-aryl, wherein the aryl ring is unsubstituted or substituted by methyl, tert-butyl, methoxy, hydroxyl, benzyloxy, chloro, fluoro, acetyl, —(CH₂)₂NHSO₂Ph, —NHCO(CH₂)₂NHCOOC(CH₃)₃ or —(CH₂)₂NHCO-3-chloro-2-methoxybenzene,
o is 0 or 1;
R′ is H,
methyl,
aryloxy, wherein the aryl ring is unsubstituted or substituted by methyl or methoxy, or
(CH═CH)_{q}-heteroaryl, wherein the heteroaryl ring is unsubstituted or substituted by methyl, acetyl, methoxy, chloro, or by chloro or fluoro substituted benzyl;
q is 0 or 1;
R⁴ is H, methyl, ethyl, —(CH₂)₂SCH₃, —NHSO₂p-Cl-Phenyl, amino, —NHCOOC(CH₃)₃, hydroxyl, phenyl, benzyl or chloro substituted benzyl;
R⁵ and R^{5′} are each independently selected from H, methyl or phenyl;
R⁶ and R^{6′} are each independently selected from H, methyl or —SCH₃;
m is 1, 2 or 3;
n is 0 or 1; and
p is 0, 1, 2 or 3;
or a pharmaceutically acceptable salt thereof.

5. The method of claim 4, wherein in the compound of formula Ia
R¹ is methyl, cyclohexyl, phenyl, morpholin-4-yl or 4-benzyloxy-phenyl;
R² is H, methyl or phenyl;
R³ is methyl, —SCH₃, acetyl, cyclopropanyl, 2,2,3,3-tetramethyl-cyclopropanyl, 2-phenyl-cyclopropanyl, cyclopent-2-enyl, cyclohexanyl, 4-tert-butyl-cyclohexanyl,
tetrahydro-furan-2-yl, pyrrolidine-2-yl, 1-tert-butyloxycarbonylpyrrolidine-2-yl piperidine-2-yl, 1-tert-butyloxycarbonylpiperidine-2-yl,
phenyl, 2-toluenyl, 3-toluenyl, 4-tert-butyl-phenyl, 4-fluoro-phenyl, 4-chloro-phenyl, 4-hydroxy-phenyl, 4-benzyloxy-phenyl, 2-methoxy-phenyl, 3-methoxy-phenyl, 4-methoxy-phenyl, —CH═C-phenyl, 2,4-dimethoxy-phenyl, 2,5-dimethoxy-phenyl, 3,4-dimethoxy-phenyl, 3,5-dimethoxy-phenyl, 4,5-dimethoxy-phenyl, 4-methoxy-2-methyl-phenyl, 4-methoxy-3-methyl-phenyl, -phenyl-4-(CH₂)₂NHSO₂Ph, -phenyl-4-NHCO(CH₂)₂NHCOOC(CH₃)₃, -phenyl-4-(CH₂)₂NHCO-3-chloro-2-methoxybenzene, naphthalen-2-yl, 6-methoxy-naphthalen-2-yl, 2-acetyl-naphthalen-1-yl, 10,11-dihydro-5H-dibenzo[a,d]cyclohepten-5-yl, 9H-fluoren-9-yl, phenoxy, 3-dimethyl-phenoxy, 2,3-dimethyl-phenoxy, 2-methoxy-phenoxy, 3-methoxy-phenoxy, naphthalene-1-yloxy, or
—CH═CH-pyridin-3-yl, indol-1-yl, 1H-indol-3-yl, 1-methyl-1H-indol-3-yl, 4-fluoro-benzyl-1H-indol-3-yl, 1-(4-chloro-benzyl)-5-methoxy-2-methyl-1H-indol-3-yl, 1-(4-chloro-benzoyl)-5-methoxy-2-methyl-1H-indol-3-yl, 2-acetyl-1,2-dihydro-isoquinolin-1-yl, 1,2,3,4-tetrahydro-isoquinoline-2-yl, (3,4-dihydro-1H-isoquinoline-2-carboxylic acid tert-butyl ester)-3-yl, 2-methyl-benzofuran-3-yl, 5-chloro-benzofuran-3-yl, benzo[b]thiophen-3-yl, or 9H-thioxanthen-9-yl;
R⁴ is H, methyl, ethyl, —(CH₂)₂SCH₃, —NHSO₂p-Cl-Phenyl, amino, —NHCOOC(CH₃)₃, hydroxyl, phenyl, benzyl or chloro substituted benzyl;
R⁵ and R^{5′} are each independently selected from H, methyl or phenyl;
R⁶ and R^{6′} are each independently selected from H, methyl or —SCH₃;
m is 1, 2 or 3;
n is 0 or 1; and
p is 0, 1, 2 or 3;
or a pharmaceutically acceptable salt thereof.

6. The method of claim 5, wherein the compound of formula Ia is selected from the group consisting of
(RS)-4-Hydroxy-5-isobutyl-3-(3-methyl-butyryl)-5H-furan-2-one;
4-Hydroxy-5-isobutyl-3-(3-methylsulfanyl-propionyl)-5H-furan-2-one;
4-Hydroxy-5-isobutyl-3-(4-methyl-pentanoyl)-5H-furan-2-one;
1-(4-Hydroxy-5-isobutyl-2-oxo-2,5-dihydro-furan-3-yl)-2-methyl-pentane-1,4-dione;
4-Hydroxy-5-isobutyl-3-(2,2,3,3-tetramethyl-cyclopropanecarbonyl)-5H-furan-2-one;
4-Hydroxy-5-isobutyl-3-(tetrahydro-furan-2-carbonyl)-5H-furan-2-one;
3-Cyclohexanecarbonyl-4-hydroxy-5-isobutyl-5H-furan-2-one;
3-(4-tert-Butyl-cyclohexanecarbonyl)-4-hydroxy-5-isobutyl-5H-furan-2-one;
3-(Cyclopent-2-enyl-acetyl)-4-hydroxy-5-isobutyl-5H-furan-2-one;
3-(2-Cyclohexyl-acetyl)-4-hydroxy-5-isobutyl-5H-furan-2-one;
3-(4-Cyclohexyl-butyryl)-4-hydroxy-5-isobutyl-5H-furan-2-one;
4-Hydroxy-5-isobutyl-3-(2-phenoxy-benzoyl)-5H-furan-2-one;
4-Hydroxy-5-isobutyl-3-phenylacetyl-5H-furan-2-one;
4-Hydroxy-5-isobutyl-3-o-tolylacetyl-5H-furan-2-one;
3-[(4-Chloro-phenyl)-acetyl]-4-hydroxy-5-isobutyl-5H-furan-2-one;
4-Hydroxy-5-isobutyl-3-[2-(4-methoxy-3-methyl-phenyl)-acetyl]-5H-furan-2-one;
3-[2-(3,5-Dimethoxy-phenyl)-acetyl]-4-hydroxy-5-isobutyl-5H-furan-2-one;
-3-[2-(2,5-Dimethoxy-phenyl)-acetyl]-4-hydroxy-5-isobutyl-5H-furan-2-one;
3-[2-(2,4-Dimethoxy-phenyl)-acetyl]-4-hydroxy-5-isobutyl-5H-furan-2-one;
3-(2-phenyl-propionyl-4-hydroxy-5-isobutyl-5H-furan-2-one;
4-Hydroxy-5-isobutyl-3-(2-phenyl-butyryl)-5H-furan-2-one;
4-Hydroxy-5-isobutyl-3-[2-(6-methoxy-naphthalen-2-yl)-propionyl]-5H-furan-2-one;
4-Hydroxy-5-isobutyl-3-(3-phenyl-propionyl)-5H-furan-2-one;
4-Hydroxy-5-isobutyl-3-(3-m-tolyl-propionyl)-5H-furan-2-one;
4-Hydroxy-5-isobutyl-3-[3-(3-methoxy-phenyl)-propionyl]-5H-furan-2-one;
4-Hydroxy-5-isobutyl-3-[3-(4-methoxy-phenyl)-propionyl]-5H-furan-2-one;
3-[3-(2,5-Dimethoxy-phenyl)-propionyl]-4-hydroxy-5-isobutyl-5H-furan-2-one;
3-[3-(4-Chloro-phenyl)-2-methyl-propionyl]-4-hydroxy-5-isobutyl-5H-furan-2-one;
3-[3-(4-tert-Butyl-phenyl)-2-methyl-propionyl]-4-hydroxy-5-isobutyl-5H-furan-2-one; and
4-Hydroxy-5-isobutyl-3-(3-phenyl-butyryl)-5H-furan-2-one.

7. The method of claim 5, wherein the compound of formula Ia is selected from the group consisting of
4-Hydroxy-5-isobutyl-3-((R)—(R)-2-phenyl-cyclopropanecarbonyl)-5H-furan-2-one;
4-Hydroxy-5-isobutyl-3-[2-(2-methoxy-phenoxy)-acetyl]-5H-furan-2-one;
4-Hydroxy-5-isobutyl-3-[2-(naphthalen-1-yloxy)-acetyl]-5H-furan-2-one;
4-Hydroxy-5-isobutyl-3-(2-phenoxy-propionyl)-5H-furan-2-one;
4-Hydroxy-5-isobutyl-3-(4-phenyl-butyryl)-5H-furan-2-one;
3-[4-(3,4-Dimethoxy-phenyl)-butyryl]-4-hydroxy-5-isobutyl-5H-furan-2-one;
4-Hydroxy-5-isobutyl-3-((Z)-2-methyl-5-pyridin-3-yl-pent-4-enoyl)-5H-furan-2-one;
4-Hydroxy-5-isobutyl-3-((Z)-2-methyl-5-phenyl-hex-4-enoyl)-5H-furan-2-one;
4-Hydroxy-3-(2-1H-indol-3-yl-acetyl)-5-isobutyl-5H-furan-2-one;
4-Hydroxy-3-(3-1H-indol-3-yl-propionyl)-5-isobutyl-5H-furan-2-one;
4-Hydroxy-5-isobutyl-3-(2-naphthalen-2-yl-acetyl)-5H-furan-2-one;
3-[2-(2-Acetyl-1,2-dihydro-isoquinolin-1-yl)-acetyl]-4-hydroxy-5-isobutyl-5H-furan-2-one;
3-Diphenylacetyl-4-hydroxy-5-isobutyl-5H-furan-2-one;
3-(3,3-Diphenyl-propionyl)-4-hydroxy-5-isobutyl-5H-furan-2-one;
4-Hydroxy-5-isobutyl-3-[(9H-thioxanthen-9-yl)-acetyl]-5H-furan-2-one;
3-[(10,11-Dihydro-5H-dibenzo[a,d]cyclohepten-5-yl)-acetyl]-4-hydroxy-5-isobutyl-5H-furan-2-one;
4-Hydroxy-3-(3-methylsulfanyl-propionyl)-5-(2-methylsulfanyl-propyl)-5H-furan-2-one;
3-Cyclopropanecarbonyl-4-hydroxy-5-(2-methylsulfanyl-propyl)-5H-furan-2-one;
4-Hydroxy-5-(2-methylsulfanyl-propyl)-3-(2,2,3,3-tetramethyl-cyclopropanecarbonyl)-5H-furan-2-one;
4-Hydroxy-5-(2-methylsulfanyl-propyl)-3-(tetrahydro-furan-2-carbonyl)-5H-furan-2-one;
3-Cyclohexanecarbonyl-4-hydroxy-5-(2-methylsulfanyl-propyl)-5H-furan-2-one;
3-(4-tert-Butyl-cyclohexanecarbonyl)-4-hydroxy-5-(2-methylsulfanyl-propyl)-5H-furan-2-one;
3-(2-Cyclohexyl-acetyl)-4-hydroxy-5-(2-methylsulfanyl-propyl)-5H-furan-2-one;
3-(4-Cyclohexyl-butyryl)-4-hydroxy-5-(2-methylsulfanyl-propyl)-5H-furan-2-one;
4-Hydroxy-5-(2-methylsulfanyl-propyl)-3-phenylacetyl-5H-furan-2-one;
4-Hydroxy-3-[2-(4-methoxy-3-methyl-phenyl)-acetyl]-5-(2-methylsulfanyl-propyl)-5H-furan-2-one;
3-[2-(3,5-Dimethoxy-phenyl)-acetyl]-4-hydroxy-5-(2-methylsulfanyl-propyl)-5H-furan-2-one;
3-[2-(2,4-Dimethoxy-phenyl)-acetyl]-4-hydroxy-5-(2-methylsulfanyl-propyl)-5H-furan-2-one;
3-[2-(2,5-Dimethoxy-phenyl)-acetyl]-4-hydroxy-5-(2-methylsulfanyl-propyl)-5H-furan-2-one; and
4-Hydroxy-5-(2-methylsulfanyl-propyl)-3-(2-naphthalen-2-yl-acetyl)-5H-furan-2-one.

8. The method of claim 5, wherein the compound of formula Ia is selected from the group consisting of
4-Hydroxy-5-(2-methylsulfanyl-propyl)-3-(2-phenyl-propionyl)-5H-furan-2-one;
4-Hydroxy-5-(2-methylsulfanyl-propyl)-3-(2-phenyl-butyryl)-5H-furan-2-one;
4-Hydroxy-3-[2-(6-methoxy-naphthalen-2-yl)-propionyl]-5-(2-methylsulfanyl-propyl)-5H-furan-2-one;
4-Hydroxy-5-(2-methylsulfanyl-propyl)-3-(3-phenyl-propionyl)-5H-furan-2-one;
4-Hydroxy-5-(2-methylsulfanyl-propyl)-3-(3-m-tolyl-propionyl)-5H-furan-2-one;
4-Hydroxy-3-[3-(3-methoxy-phenyl)-propionyl]-5-(2-methylsulfanyl-propyl)-5H-furan-2-one;
4-Hydroxy-3-[3-(4-methoxy-phenyl)-propionyl]-5-(2-methylsulfanyl-propyl)-5H-furan-2-one;
3-[3-(2,5-Dimethoxy-phenyl)-propionyl]-4-hydroxy-5-(2-methylsulfanyl-propyl)-5H-furan-2-one;
3-[3-(4-tert-Butyl-phenyl)-2-methyl-propionyl]-4-hydroxy-5-(2-methylsulfanyl-propyl)-5H-furan-2-one;
4-Hydroxy-5-(2-methylsulfanyl-propyl)-3-(3-phenyl-butyryl)-5H-furan-2-one;
4-Hydroxy-5-(2-methylsulfanyl-propyl)-3-((R)—(R)-2-phenyl-cyclopropanecarbonyl)-5H-furan-2-one;
4-Hydroxy-3-[2-(2-methoxy-phenoxy)-acetyl]-5-(2-methylsulfanyl-propyl)-5H-furan-2-one;
3-[2-(2,3-Dimethyl-phenoxy)-acetyl]-4-hydroxy-5-(2-methylsulfanyl-propyl)-5H-furan-2-one;
4-Hydroxy-5-(2-methylsulfanyl-propyl)-3-(2-phenoxy-propionyl)-5H-furan-2-one;
4-Hydroxy-5-(2-methylsulfanyl-propyl)-3-(2-phenoxy-butyryl)-5H-furan-2-one;
4-Hydroxy-5-(2-methylsulfanyl-propyl)-3-[2-(naphthalen-1-yloxy)-acetyl]-5H-furan-2-one;
4-Hydroxy-5-(2-methylsulfanyl-propyl)-3-(4-phenyl-butyryl)-5H-furan-2-one;
3-[4-(3,4-Dimethoxy-phenyl)-butyryl]-4-hydroxy-5-(2-methylsulfanyl-propyl)-5H-furan-2-one;
4-Hydroxy-3-[(H-indol-3-yl)-acetyl]-5-(2-methylsulfanyl-propyl)-5H-furan-2-one;
4-Hydroxy-3-(3-1H-indol-3-yl-propionyl)-5-(2-methylsulfanyl-propyl)-5H-furan-2-one;
3-[2-(2-Acetyl-1,2-dihydro-isoquinolin-1-yl)-acetyl]-4-hydroxy-5-(2-methylsulfanyl-propyl)-5H-furan-2-one;
3-Diphenylacetyl-4-hydroxy-5-(2-methylsulfanyl-propyl)-5H-furan-2-one;
3-(3,3-Diphenyl-propionyl)-4-hydroxy-5-(2-methylsulfanyl-propyl)-5H-furan-2-one;
4-Hydroxy-5-(2-methylsulfanyl-propyl)-3-(2-9H-thioxanthen-9-yl-acetyl)-5H-furan-2-one;
3-(2-10,11-Dihydro-5H-dibenzo[a,d]cyclohepten-5-yl-acetyl)-4-hydroxy-5-(2-methylsulfanyl-propyl)-5H-furan-2-one;
3-Cyclohexanecarbonyl-5-cyclohexylmethyl-4-hydroxy-5H-furan-2-one;
3-Cyclohexylacetyl-5-cyclohexylmethyl-4-hydroxy-5H-furan-2-one;
5-Cyclohexylmethyl-3-(3-cyclohexyl-propionyl)-4-hydroxy-5H-furan-2-one; and
3-(4-Cyclohexyl-butyryl)-5-cyclohexylmethyl-4-hydroxy-5H-furan-2-one.

9. The method of claim 5, wherein the compound of formula Ia is selected from the group consisting of
4-Chloro-N-[3-cyclohexyl-1-(5-cyclohexylmethyl-4-hydroxy-2-oxo-2,5-dihydro-furan-3-carbonyl)-propyl]-benzenesulfonamide;
5-Cyclohexylmethyl-3-(5-cyclohexyl-pentanoyl)-4-hydroxy-5H-furan-2-one;
5-Cyclohexylmethyl-4-hydroxy-3-(2-methyl-3-phenyl-propionyl)-5H-furan-2-one;
3-[3-(4-tert-Butyl-phenyl)-2-methyl-propionyl]-5-cyclohexylmethyl-4-hydroxy-5H-furan-2-one;
3-[3-(4-Benzyloxy-phenyl)-2-methyl-propionyl]-5-cyclohexylmethyl-4-hydroxy-5H-furan-2-one;
(2-{4-[3-(5-Cyclohexylmethyl-4-hydroxy-2-oxo-2,5-dihydro-furan-3-yl)-2-methyl-3-oxo-propyl]-phenylcarbamoyl}-ethyl)-carbamic acid tert-butyl ester;
N-(2-{4-[3-(5-Cyclohexylmethyl-4-hydroxy-2-oxo-2,5-dihydro-furan-3-yl)-2-methyl-3-oxo-propyl]-phenyl}-ethyl)-benzenesulfonamide;
5-Chloro-N-(2-{4-[3-(5-cyclohexylmethyl-4-hydroxy-2-oxo-2,5-dihydro-furan-3-yl)-2-methyl-3-oxo-propyl]-phenyl}-ethyl)-2-methoxy-benzamide;
[1-(4-Benzyloxy-benzyl)-2-(5-cyclohexylmethyl-4-hydroxy-2-oxo-2,5-dihydro-furan-3-yl)-2-oxo-ethyl]-carbamic acid tert-butyl ester;
[2-(5-Cyclohexylmethyl-4-hydroxy-2-oxo-2,5-dihydro-furan-3-yl)-1-(4-hydroxy-benzyl)-2-oxo-ethyl]-carbamic acid tert-butyl ester;
3-[2-Amino-3-(4-hydroxy-phenyl)-propionyl]-5-cyclohexylmethyl-4-hydroxy-5H-furan-2-one;
compound with trifluoro-acetic acid;
5-Cyclohexylmethyl-4-hydroxy-3-[(2-methoxy-phenoxy)-acetyl]-5H-furan-2-one;
5-Cyclohexylmethyl-4-hydroxy-3-[(1H-indol-3-yl)-acetyl]-5H-furan-2-one;
5-Cyclohexylmethyl-4-hydroxy-3-[(1-methyl-1H-indol-3-yl)-acetyl]-5H-furan-2-one;
5-Cyclohexylmethyl-3-{[1-(4-fluoro-benzyl)-1H-indol-3-yl]-acetyl}-4-hydroxy-5H-furan-2-one;
3-{[1-(4-Chloro-benzyl)-5-methoxy-2-methyl-1H-indol-3-yl]-acetyl}-5-cyclohexylmethyl-4-hydroxy-5H-furan-2-one;
3-{[1-(4-Chloro-benzoyl)-5-methoxy-2-methyl-1H-indol-3-yl]-acetyl}-5-cyclohexylmethyl-4-hydroxy-5H-furan-2-one;
5-Cyclohexylmethyl-4-hydroxy-3-(indol-1-yl-acetyl)-5H-furan-2-one;
5-Cyclohexylmethyl-4-hydroxy-3-(3-1H-indol-3-yl-propionyl)-5H-furan-2-one;
5-Cyclohexylmethyl-4-hydroxy-3-[(2-methyl-benzofuran-3-yl)-acetyl]-5H-furan-2-one;
3-[(5-Chloro-benzofuran-3-yl)-acetyl]-5-cyclohexylmethyl-4-hydroxy-5H-furan-2-one;
3-(Benzo[b]thiophen-3-yl-acetyl)-5-cyclohexylmethyl-4-hydroxy-5H-furan-2-one;
5-Cyclohexylmethyl-3-(3,3-diphenyl-propionyl)-4-hydroxy-5H-furan-2-one;
5-Cyclohexylmethyl-3-(2,3-diphenyl-propionyl)-4-hydroxy-5H-furan-2-one;
5-Cyclohexylmethyl-3-[3-(4-fluoro-phenyl)-2-phenyl-propionyl]-4-hydroxy-5H-furan-2-one;
3-(2-Benzyl-3-phenyl-propionyl)-5-cyclohexylmethyl-4-hydroxy-5H-furan-2-one;
3-[2-(4-Chloro-benzyl)-3-(4-chloro-phenyl)-propionyl]-5-cyclohexylmethyl-4-hydroxy-5H-furan-2-one;
5-Cyclohexylmethyl-3-[(9H-fluoren-9-yl)-acetyl]-4-hydroxy-5H-furan-2-one;
3-(Carbazol-9-yl-acetyl)-5-cyclohexylmethyl-4-hydroxy-5H-furan-2-one; and
5-Benzyl-3-cyclohexanecarbonyl-4-hydroxy-5H-furan-2-one.

10. The method of claim 5, wherein the compound of formula Ia is selected from the group consisting of
5-Benzyl-3-[3-(4-tert-butyl-phenyl)-2-methyl-propionyl]-4-hydroxy-5H-furan-2-one;
5-Benzyl-4-hydroxy-3-[(2-methoxy-phenoxy)-acetyl]-5H-furan-2-one;
5-Benzyl-3-(4-cyclohexyl-butyryl)-4-hydroxy-5H-furan-2-one;
5-Benzyl-4-hydroxy-3-[(1H-indol-3-yl)-acetyl]-5H-furan-2-one;
5-Benzyl-3-(3,3-diphenyl-propionyl)-4-hydroxy-5H-furan-2-one;
5-Benzyl-3-[(9H-fluoren-9-yl)-acetyl]-4-hydroxy-5H-furan-2-one;
Rac-4-Hydroxy-3-(3-methyl-sulfanyl-propionyl)-5-phenethyl-5H-furan-2-one;
Rac-3-(2 (R,S),4-dimethyl-pentanoyl)-4-hydroxy-5-phenethyl-5H-furan-2-one;
Rac-4-hydroxy-3-(2 (R,S)-methyl-hexanoyl)-5-phenethyl-5H-furan-2-one;
Rac-3-cyclopropane-carbonyl-4-hydroxy-5-phenethyl-5H-furan-2-one;
Rac-3-cyclohexane-carbonyl-4-hydroxy-5-phenethyl-5H-furan-2-one;
Rac-3-(2-cyclohexyl-acetyl)-4-hydroxy-5-phenethyl-5H-furan-2-one;
Rac-3-(4-cyclohexyl-butyryl)-4-hydroxy-5-phenethyl-5H-furan-2-one;
Rac-4-hydroxy-5-phenethyl-3-phenylacetyl-5H-furan-2-one;
Rac-4-hydroxy-5-phenethyl-3-(2-o-tolyl-acetyl)-5H-furan-2-one;
Rac-4-hydroxy-5-phenethyl-3-(2(R,S)-phenyl-propionyl)-5H-furan-2-one;
Rac-4-hydroxy-5-phenethyl-3-(2 (R,S)-phenyl-butyryl)-5H-furan-2-one;
Rac-3-[2-(2,5-dimethoxy-phenyl)-acetyl]-4-hydroxy-5-phenethyl-5H-furan-2-one;
Rac-3-[2-(2,4-dimethoxy-phenyl)-acetyl]-4-hydroxy-5-phenethyl-5H-furan-2-one;
Rac-3-[2-(3,5-dimethoxy-phenyl)-acetyl]-4-hydroxy-5-phenethyl-5H-furan-2-one;
Rac-4-hydroxy-5-phenethyl-3-(3-phenyl-propionyl)-5H-furan-2-one;
4-Hydroxy-5-phenethyl-3-((R)—(R)-2-phenyl-cyclopropanecarbonyl)-5H-furan-2-one;
Rac-4-hydroxy-5-phenethyl-3-(3(R,S)-phenyl-butyryl)-5H-furan-2-one;
Rac-4-hydroxy-3-(2 (R,S)-hydroxy-3-phenyl-propionyl)-5-phenethyl-5H-furan-2-one;
Rac-4-hydroxy-5-phenethyl-3-(3-m-tolyl-propionyl)-5H-furan-2-one;
Rac-4-hydroxy-3-[2-(2-methoxy-phenoxy)-acetyl]-5-phenethyl-5H-furan-2-one;
Rac-4-hydroxy-3-[3-(3-methoxy-phenyl)-propionyl]-5-phenethyl-5H-furan-2-one;
Rac-4-hydroxy-3-[3-(4-methoxy-phenyl)-propionyl]-5-phenethyl-5H-furan-2-one;
Rac-3-[3-(2,5-dimethoxy-phenyl)-propionyl]-4-hydroxy-5-phenethyl-5H-furan-2-one;
Rac-3-[3-(4-tert-butyl-phenyl)-2 (R,S)-methyl-propionyl]-4-hydroxy-5-phenethyl-5H-furan-2-one;
Rac-3-[3-(4-chloro-phenyl)-2 (R,S)-methyl-propionyl]-4-hydroxy-5-phenethyl-5H-furan-2-one;
4-Hydroxy-5-phenethyl-3-(4-phenyl-butyryl)-5H-furan-2-one;
3-[4-(3,4-Dimethoxy-phenyl)-butyryl]-4-hydroxy-5-phenethyl-5H-furan-2-one;
4-Hydroxy-3-(2-naphthalen-2-yl-acetyl)-5-phenethyl-5H-furan-2-one; and
Rac-4-hydroxy-3-[2 (R,S)—(6-methoxy-naphthalen-2-yl)-propionyl]-5-phenethyl-5H-furan-2-one.

11. The method of claim 5, wherein the compound of formula Ia is selected from the group consisting of
3-[(2-Acetyl-naphthalen-1-yl)-acetyl]-4-hydroxy-5-phenethyl-5H-furan-2-one;
3-[2-(2-Acetyl-1,2-dihydro-isoquinolin-1-yl)-acetyl]-4-hydroxy-5-phenethyl-5H-furan-2-one;
4-Hydroxy-3-(2-1H-indol-3-yl-acetyl)-5-phenethyl-5H-furan-2-one;
Rac-4-hydroxy-3-(3-1H-indol-3-yl-propionyl)-5-phenethyl-5H-furan-2-one;
Rac-4-hydroxy-3-[2-(naphthalen-1-yloxy)-acetyl]-5-phenethyl-5H-furan-2-one;
Rac-3-(3,3-diphenyl-propionyl)-4-hydroxy-5-phenethyl-5H-furan-2-one;
Rac-3-(2-10,11-dihydro-5H-dibenzo[a,d]cyclohepten-5-yl-acetyl)-4-hydroxy-5-phenethyl-5H-furan-2-one;
Rac-4-hydroxy-5-phenethyl-3-(2-9H-thioxanthen-9-yl-acetyl)-5H-furan-2-one;
Rac-3-(2-9H-fluoren-9-yl-acetyl)-4-hydroxy-5-phenethyl-5H-furan-2-one;
Rac-[2-(4-hydroxy-2-oxo-5-phenethyl-2,5-dihydro-furan-3-yl)-1(R,S)-methyl-2-oxo-ethyl]-carbamic acid tert-butyl ester;
Rac-3-(2 (R,S)-amino-propionyl)-4-hydroxy-5-phenethyl-5H-furan-2-one;
[1(R)-Benzyl-2-(4-hydroxy-2-oxo-5(R,S)-phenethyl-2,5-di-hydro-furan-3-yl)-2-oxo-ethyl]-carbamic acid tert-butylester;
3-(2(R)-Amino-3-phenyl-propionyl)-4-hydroxy-5(R,S)-phenethyl-5H-furan-2-one;
Rac-[1(R,S)-(4-benzyloxy-benzyl)-2-(4-hydroxy-2-oxo-5-phenethyl-2,5-dihydro-furan-3-yl)-2-oxo-ethyl]-carbamic acid tert-butyl ester;
[1(S)-(4-Benzyloxy-benzyl)-2-(4-hydroxy-2-oxo-5(R,S)-phenethyl-2,5-dihydro-furan-3-yl)-2-oxo-ethyl]-carbamic acid tert-butyl ester;
[1(R)-(4-Benzyloxy-benzyl)-2-(4-hydroxy-2-oxo-5(R,S)-phenethyl-2,5-dihydro-furan-3-yl)-2-oxo-ethyl]-carbamic acid tert-butyl ester;
Rac-3-[2 (R,S)-amino-3-(4-benzyloxy-phenyl)-propionyl]-4-hydroxy-5-phenethyl-5H-furan-2-one;
2-(4-Hydroxy-2-oxo-5(R,S)-phenethyl-2,5-dihydro-furan-3-carbonyl)-pyrrolidine-1(S)-carboxylic acid tert-butyl ester;
4-Hydroxy-5(R,S)-phenethyl-3-(pyrrolidine-2(S)-carbonyl)-5H-furan-2-one;
Rac-2(R,S)-(4-Hydroxy-2-oxo-5-phenethyl-2,5-dihydro-furan-3-carbonyl)-piperidine-1-carboxylic acid tert-butyl ester;
Rac-4-hydroxy-5-phenethyl-3 (R,S)-(piperidine-2-carbonyl)-5H-furan-2-one;
Rac-3 (R,S)-(4-hydroxy-2-oxo-5-phenethyl-2,5-dihydro-furan-3-carbonyl)-3,4-dihydro-1H-iso-quinoline-2-carboxylic acid tert-butyl ester;
Rac-4-hydroxy-5-phenethyl-3 (R,S)-(1,2,3,4-tetrahydro-isoquinoline-3-carbonyl)-5H-furan-2-one;
3-4-Cyclohexanecarbonyl-4-hydroxy-5-(3-phenyl-propyl)-5H-furan-2-one;
3-(4-Cyclohexyl-butyryl)-4-hydroxy-5-(3-phenyl-propyl)-5H-furan-2-one;
3-[3-(4-tert-Butyl-phenyl)-2-methyl-propionyl]-4-hydroxy-5-(3-phenyl-propyl)-5H-furan-2-one;
4-Hydroxy-3-[(2-methoxy-phenoxy)-acetyl]-5-(3-phenyl-propyl)-5H-furan-2-one;
4-Hydroxy-3-[(1H-indol-3-yl)-acetyl]-5-(3-phenyl-propyl)-5H-furan-2-one;
3-(3,3-Diphenyl-propionyl)-4-hydroxy-5-(3-phenyl-propyl)-5H-furan-2-one; and
3-[(9H-Fluoren-9-yl)-acetyl]-4-hydroxy-5-(3-phenyl-propyl)-5H-furan-2-one.

12. The method of claim 5, wherein the compound of formula Ia is selected from the group consisting of
4-Hydroxy-3-(3-methylsulfanyl-propionyl)-5-(3-morpholin-4-yl-propyl)-5H-furan-2-one;
3-Cyclopropanecarbonyl-4-hydroxy-5-(3-morpholin-4-yl-propyl)-5H-furan-2-one;
4-Hydroxy-5-(3-morpholin-4-yl-propyl)-3-(2,2,3,3-tetramethyl-cyclopropanecarbonyl)-5H-furan-2-one;
4-Hydroxy-5-(3-morpholin-4-yl-propyl)-3-(tetrahydro-furan-2-carbonyl)-5H-furan-2-one;
3-Cyclohexanecarbonyl-4-hydroxy-5-(3-morpholin-4-yl-propyl)-5H-furan-2-one;
3-(2-Cyclohexyl-acetyl)-4-hydroxy-5-(3-morpholin-4-yl-propyl)-5H-furan-2-one;
3-(4-Cyclohexyl-butyryl)-4-hydroxy-5-(3-morpholin-4-yl-propyl)-5H-furan-2-one;
4-Hydroxy-5-(3-morpholin-4-yl-propyl)-3-phenylacetyl-5H-furan-2-one;
4-Hydroxy-5-(3-morpholin-4-yl-propyl)-3-(2-phenyl-propionyl)-5H-furan-2-one;
3-[2-(3,5-Dimethoxy-phenyl)-acetyl]-4-hydroxy-5-(3-morpholin-4-yl-propyl)-5H-furan-2-one;
3-[2-(2,5-Dimethoxy-phenyl)-acetyl]-4-hydroxy-5-(3-morpholin-4-yl-propyl)-5H-furan-2-one;
3-[2-(2,4-Dimethoxy-phenyl)-acetyl]-4-hydroxy-5-(3-morpholin-4-yl-propyl)-5H-furan-2-one;
4-Hydroxy-3-[2-(4-methoxy-2-methyl-phenyl)-acetyl]-5-(3-morpholin-4-yl-propyl)-5H-furan-2-one;
4-Hydroxy-3-[3-(4-methoxy-phenyl)-propionyl]-5-(3-morpholin-4-yl-propyl)-5H-furan-2-one;
4-Hydroxy-5-(3-morpholin-4-yl-propyl)-3-(3-phenyl-butyryl)-5H-furan-2-one;
3-[3-(2,5-Dimethoxy-phenyl)-propionyl]-4-hydroxy-5-(3-morpholin-4-yl-propyl)-5H-furan-2-one;
4-Hydroxy-5-(3-morpholin-4-yl-propyl)-3-(3-m-tolyl-propionyl)-5H-furan-2-one;
4-Hydroxy-3-[3-(3-methoxy-phenyl)-propionyl]-5-(3-morpholin-4-yl-propyl)-5H-furan-2-one;
4-Hydroxy-3-[2-(3-methoxy-phenoxy)-acetyl]-5-(3-morpholin-4-yl-propyl)-5H-furan-2-one;
4-Hydroxy-5-(3-morpholin-4-yl-propyl)-3-(2-m-tolyloxy-acetyl)-5H-furan-2-one;
4-Hydroxy-3-[2-(2-methoxy-phenoxy)-acetyl]-5-(3-morpholin-4-yl-propyl)-5H-furan-2-one;
3-[2-(2,3-Dimethyl-phenoxy)-acetyl]-4-hydroxy-5-(3-morpholin-4-yl-propyl)-5H-furan-2-one;
4-Hydroxy-5-(3-morpholin-4-yl-propyl)-3-(4-phenyl-butyryl)-5H-furan-2-one;
4-Hydroxy-5-(3-morpholin-4-yl-propyl)-3-(2-naphthalen-2-yl-acetyl)-5H-furan-2-one;
4-Hydroxy-5-(3-morpholin-4-yl-propyl)-3-[2-(naphthalen-1-yloxy)-acetyl]-5H-furan-2-one;
4-Hydroxy-3-(2-1H-indol-3-yl-acetyl)-5-(3-morpholin-4-yl-propyl)-5H-furan-2-one;
4-Hydroxy-3-(3-1H-indol-3-yl-propionyl)-5-(3-morpholin-4-yl-propyl)-5H-furan-2-one;
3-[2-(2-Acetyl-1,2-dihydro-isoquinolin-1-yl)-acetyl]-4-hydroxy-5-(3-morpholin-4-yl-propyl)-5H-furan-2-one;
3-(3,3-Diphenyl-propionyl)-4-hydroxy-5-(3-morpholin-4-yl-propyl)-5H-furan-2-one; and
4-Hydroxy-5-(3-morpholin-4-yl-propyl)-3-(2-9H-thioxanthen-9-yl-acetyl)-5H-furan-2-one.

13. The compound of formula Ia according to claim 5, which is
5-[2-(4-Benzyloxy-phenyl)-ethyl]-3-(4-cyclohexyl-butyryl)-4-hydroxy-5H-furan-2-one;
3-Cyclohexanecarbonyl-4-hydroxy-5-methyl-5-phenethyl-5H-furan-2-one;
3-(4-Cyclohexyl-butyryl)-4-hydroxy-5-methyl-5-phenethyl-5H-furan-2-one;
3-[3-(4-tert-Butyl-phenyl)-2-methyl-propionyl]-4-hydroxy-5-methyl-5-phenethyl-5H-furan-2-one;
4-Hydroxy-3-[(2-methoxy-phenoxy)-acetyl]-5-methyl-5-phenethyl-5H-furan-2-one;
4-Hydroxy-3-[(H-indol-3-yl)-acetyl]-5-methyl-5-phenethyl-5H-furan-2-one;
3-(3,3-Diphenyl-propionyl)-4-hydroxy-5-methyl-5-phenethyl-5H-furan-2-one;
3-[(9H-Fluoren-9-yl)-acetyl]-4-hydroxy-5-methyl-5-phenethyl-5H-furan-2-one;
3-Cyclohexanecarbonyl-4-hydroxy-5-phenethyl-5-phenyl-5H-furan-2-one;
4-Hydroxy-3-[(2-methoxy-phenoxy)-acetyl]-5-phenethyl-5-phenyl-5H-furan-2-one;
4-Hydroxy-3-[(1H-indol-3-yl)-acetyl]-5-phenethyl-5-phenyl-5H-furan-2-one;
3-(3,3-Diphenyl-propionyl)-4-hydroxy-5-phenethyl-5-phenyl-5H-furan-2-one;
3-[(9H-Fluoren-9-yl)-acetyl]-4-hydroxy-5-phenethyl-5-phenyl-5H-furan-2-one;
Rac-4-hydroxy-5-isobutyl-3-[(9H-thioxanthen-9-yl)-acetyl]-5H-furan-2-one;
3-[3-(4-tert-Butyl-phenyl)-2(R,S)-methyl-propionyl]-5(R,S)-cyclohexylmethyl-4-hydroxy-5H-furan-2-one;
5-Chloro-N-(2-{4-[3-(5(R,S)-cyclohexylmethyl-4-hydroxy-2-oxo-2,5-dihydro-furan-3-yl)-2 (R,S)-methyl-3-oxo-propyl]-phenyl}-ethyl)-2-methoxy-benzamide;
Rac-5-cyclohexylmethyl-4-hydroxy-3-[(H-indol-3-yl)-acetyl]-5H-furan-2-one;
Rac-5-cyclohexylmethyl-3-{[1-(4-fluoro-benzyl)-1H-indol-3-yl]-acetyl}-4-hydroxy-5H-furan-2-one;
Rac-5-cyclohexylmethyl-3-[(9H-fluoren-9-yl)-acetyl]-4-hydroxy-5H-furan-2-one;
Rac-3-(carbazol-9-yl-acetyl)-5-cyclohexylmethyl-4-hydroxy-5H-furan-2-one;
5(R,S)-Benzyl-3-[3-(4-tert-butyl-phenyl)-2 (R,S)-methyl-propionyl]-4-hydroxy-5H-furan-2-one;
Rac-4-hydroxy-3-[(2-methoxy-phenoxy)-acetyl]-5-phenethyl-5H-furan-2-one;
Rac-4-hydroxy-3-[(1H-indol-3-yl)-acetyl]-5-phenethyl-5H-furan-2-one;
Rac-3-(3,3-diphenyl-propionyl)-4-hydroxy-5-phenethyl-5H-furan-2-one;
Rac-4-hydroxy-3-[(H-indol-3-yl)-acetyl]-5-(3-phenyl-propyl)-5H-furan-2-one; and
Rac-3-[(9H-fluoren-9-yl)-acetyl]-4-hydroxy-5-methyl-5-phenethyl-5H-furan-2-one.

14. The method of claim 1, wherein the compound of formula I has the formula Ib 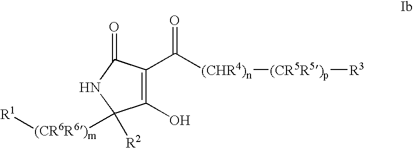
or a pharmaceutically acceptable salt thereof,
with the exception that the compound is not 3-acetyl-4-hydroxy-5-isobutyl-1,5-dihydro-pyrrol-2-one.

15. The method of claim 14, wherein in the compound of formula Ib
R¹ is aryl;
R² is H;
R³ is —SCH₃, 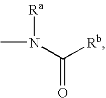
wherein R^{a} is H or lower alkyl, R^{b} is lower alkyl, heteroaryl, —OC(CH₃)₃ or aryl, wherein the aryl ring is unsubstituted or substituted by lower alkyl,
cycloalkyl, wherein the cycloalkyl ring is unsubstituted or substituted by lower alkyl,
heterocycloalkyl, wherein the heterocycloalkyl ring is unsubstituted or substituted by —COOC(CH₃)₃,
aryl, wherein the aryl ring is unsubstituted or substituted by lower alkyl, alkoxy, benzyloxy or for the non aromatic part of fused ring system also by oxo,
aryloxy, wherein the aryl ring is unsubstituted substituted by alkoxy, or
heteroaryl, wherein the heteroaryl ring is unsubstituted or substituted by lower alkyl, —COOC(CH₃)₃ or by halogen substituted benzyl, or for the non aromatic part of fused ring system also by oxo;
R⁴ is H, lower alkyl, —NHCOCH₃, amino, —NHCOOC(CH₃)₃, aryl or benzyl;
R⁵ and R^{5′} are each H;
R⁶ and R^{6′} are each H;
m is 2;
n is 0 or 1; and
p is 0, 1, 2 or 3;
or a pharmaceutically acceptable salt thereof.

16. The method of claim 15, wherein in the compound of formula Ib
R¹ is phenyl;
R² is H;
R³ is —SCH₃, 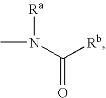
wherein R^{a} is H or methyl, R^{b} is methyl, 1H-pyrrol-3-yl, —OC(CH₃)₃ or aryl, wherein the aryl ring is unsubstituted or substituted by methyl,
cycloalkyl, wherein the cycloalkyl ring is unsubstituted or substituted by methyl,
heterocycloalkyl, wherein the heterocycloalkyl ring is unsubstituted or substituted by —COOC(CH₃)₃,
aryl, wherein the aryl ring is unsubstituted or substituted by methyl, tert-butyl, methoxy, benzyloxy or for the non aromatic part of fused ring system also by oxo,
aryloxy, wherein the aryl ring is substituted by methoxy, or
heteroaryl, wherein the heteroaryl ring is unsubstituted or substituted by methyl, —COOC(CH₃)₃ or by 4-fluoro-benzyl-1-yl, or for the non aromatic part of fused ring system also by oxo;
R⁴ is H, methyl, —NHCOCH₃, amino, —NHCOOC(CH₃)₃, phenyl or benzyl;
R⁵ and R^{5′} are each H;
R⁶ and R^{6′} are each H;
m is 2;
n is 0 or 1; and
p is 0, 1, 2 or 3;
or a pharmaceutically acceptable salt thereof.

17. The method of claim 16, wherein in the compound of formula Ib
R¹ is phenyl;
R² is H;
R³ is —SCH₃, —NHCOCH₃, —NHCO-phenyl, —NHCO-(4-methyl-phenyl), —NHCO-(2,5-dihydro-1H-pyrrol-3-yl), NHCOOC(CH₃)₃,
cyclopropanyl, 1-methyl-cyclopropanyl, cyclohexanyl,
1-tert-butyloxycarbonylpyrrolidine-2-yl, 1-ter-butyloxycarbonylpiperidine-2-yl, tetrahydro-furan-2-yl,
phenyl, toluenyl, 4-tert-butyl-phenyl, 2-methoxy-phenyl, 3-methoxy-phenyl, 4-benzoxy-phenyl, 3,4-dimethoxy-phenyl, naphthalene-2-yl, 6-methoxy-naphthalen-2-yl, 3-oxo-indan-1-yl,
2-methyl-phenoxyl, or
1,2,5-trimethyl-1H-pyrrole-3-yl, 5-methyl-pyrazine-2-yl, 5-methyl-2,4-dioxo-1H-pyrimidine-1-yl, 3-methyl-furan-2-yl, indol-1-yl, 1H-indol-3-yl, (4-fluoro-benzyl)-1H-indol-3-yl, isoquinoline-3-yl, 3,4-dihydro-1H-isoquinoline-2-carboxylic acid tert-butyl ester, thieno[2,3-c]pyridine-7-yl, benzo[1,2,3]thiadiazole-5-yl, 2,3-dihydro-benzofuran-7-yl, 2-benzo[b]thiophen-3-yl, or carbazol-9-yl,
R⁴ is H, methyl, —NHCOCH₃, amino, —NHCOOC(CH₃)₃, phenyl or benzyl;
R⁵ and R^{5′} are each H;
R⁶ and R^{6′} are each H;
m is 2;
n is 0 or 1; and
p is 0, 1, 2 or 3;
or a pharmaceutically acceptable salt thereof.

18. The method of claim 17, wherein the compound of formula Ib is selected from the group consisting of
4-Hydroxy-3-(3-methylsulfanyl-propionyl)-5-phenethyl-1,5-dihydro-pyrrol-2-one;
3-Cyclopropanecarbonyl-4-hydroxy-5-phenethyl-1,5-dihydro-pyrrol-2-one;
4-Hydroxy-3-(1-methyl-cyclopropanecarbonyl)-5-phenethyl-1,5-dihydro-pyrrol-2-one;
4-Hydroxy-5-phenethyl-3-(tetrahydro-furan-2-carbonyl)-1,5-dihydro-pyrrol-2-one;
3-(4-Cyclohexyl-butyryl)-4-hydroxy-5-phenethyl-1,5-dihydro-pyrrol-2-one;
4-Hydroxy-5-phenethyl-3-(thieno[2,3-c]pyridine-7-carbonyl)-1,5-dihydro-pyrrol-2-one;
4-Hydroxy-3-(5-methyl-pyrazine-2-carbonyl)-5-phenethyl-1,5-dihydro-pyrrol-2-one;
4-Hydroxy-3-(isoquinoline-3-carbonyl)-5-phenethyl-1,5-dihydro-pyrrol-2-one;
3-(Benzo[1,2,3]thiadiazole-5-carbonyl)-4-hydroxy-5-phenethyl-1,5-dihydro-pyrrol-2-one;
4-Hydroxy-3-(3-methyl-furan-2-carbonyl)-5-phenethyl-1,5-dihydro-pyrrol-2-one;
3-(2,3-Dihydro-benzofuran-7-carbonyl)-4-hydroxy-5-phenethyl-1,5-dihydro-pyrrol-2-one;
4-Hydroxy-5-phenethyl-3-(1,2,5-trimethyl-1H-pyrrole-3-carbonyl)-1,5-dihydro-pyrrol-2-one;
4-Hydroxy-5-phenethyl-3-phenylacetyl-1,5-dihydro-pyrrol-2-one;
4-Hydroxy-3-(2-naphthalen-2-yl-acetyl)-5-phenethyl-1,5-dihydro-pyrrol-2-one;
4-Hydroxy-3-[2-(3-oxo-indan-1-yl)-acetyl]-5-phenethyl-1,5-dihydro-pyrrol-2-one;
1-[2-(4-Hydroxy-2-oxo-5-phenethyl-2,5-dihydro-1H-pyrrol-3-yl)-2-oxo-ethyl]-5-methyl-1H-pyrimidine-2,4-dione;
4-Hydroxy-5-phenethyl-3-(2-phenyl-propionyl)-1,5-dihydro-pyrrol-2-one;
4-Hydroxy-3-[2-(6-methoxy-naphthalen-2-yl)-propionyl]-5-phenethyl-1,5-dihydro-pyrrol-2-one;
4-Hydroxy-5-phenethyl-3-(3-m-tolyl-propionyl)-1,5-dihydro-pyrrol-2-one;
4-Hydroxy-3-[3-(3-methoxy-phenyl)-propionyl]-5-phenethyl-1,5-dihydro-pyrrol-2-one;
4-Hydroxy-3-[3-(2-methoxy-phenyl)-propionyl]-5-phenethyl-1,5-dihydro-pyrrol-2-one;
4-Hydroxy-3-[3-(4-methoxy-phenyl)-propionyl]-5-phenethyl-1,5-dihydro-pyrrol-2-one;
3-[3-(4-tert-Butyl-phenyl)-2-methyl-propionyl]-4-hydroxy-5-phenethyl-1,5-dihydro-pyrrol-2-one;
4-Hydroxy-3-[(2-methoxy-phenoxy)-acetyl]-5-phenethyl-1,5-dihydro-pyrrol-2-one;
4-Hydroxy-5-phenethyl-3-(4-phenyl-butyryl)-1,5-dihydro-pyrrol-2-one;
3-[4-(3,4-Dimethoxy-phenyl)-butyryl]-4-hydroxy-5-phenethyl-1,5-dihydro-pyrrol-2-one;
N-[2-(4-Hydroxy-2-oxo-5-phenethyl-2,5-dihydro-1H-pyrrol-3-yl)-2-oxo-ethyl]-acetamide;
N-[1-(4-Hydroxy-2-oxo-5-phenethyl-2,5-dihydro-1H-pyrrole-3-carbonyl)-3-methylsulfanyl-propyl]-acetamide;
N-[2-(4-Hydroxy-2-oxo-5-phenethyl-2,5-dihydro-1H-pyrrol-3-yl)-2-oxo-ethyl]-N-methyl-benzamide; and
N-[2-(4-Hydroxy-2-oxo-5-phenethyl-2,5-dihydro-1H-pyrrol-3-yl)-2-oxo-ethyl]-4-methyl-benzamide.

19. The method of claim 17, wherein the compound of formula Ib is selected from the group consisting of
N-[2-(4-Hydroxy-2-oxo-5-phenethyl-2,5-dihydro-1H-pyrrol-3-yl)-2-oxo-ethyl]-nicotinamide;
[2-(4-Hydroxy-2-oxo-5-phenethyl-2,5-dihydro-1H-pyrrol-3-yl)-1-methyl-2-oxo-ethyl]-carbamic acid tert-butyl ester;
[1-Benzyl-2-(4-hydroxy-2-oxo-5-phenethyl-2,5-dihydro-1H-pyrrol-3-yl)-2-oxo-ethyl]-carbamic acid tert-butyl ester;
2-(4-Hydroxy-2-oxo-5-phenethyl-2,5-dihydro-1H-pyrrole-3-carbonyl)-pyrrolidine-1-carboxylic acid tert-butyl ester;
2-(4-Hydroxy-2-oxo-5-phenethyl-2,5-dihydro-1H-pyrrole-3-carbonyl)-piperidine-1-carboxylic acid tert-butyl ester;
3-(4-Hydroxy-2-oxo-5-phenethyl-2,5-dihydro-1H-pyrrole-3-carbonyl)-3,4-dihydro-1H-isoquinoline-2-carboxylic acid tert-butyl ester;
[1-(4-Benzyloxy-benzyl)-2-(4-hydroxy-2-oxo-5-phenethyl-2,5-dihydro-1H-pyrrol-3-yl)-2-oxo-ethyl]-carbamic acid tert-butyl ester;
3-[2-Amino-3-(4-benzyloxy-phenyl)-propionyl]-4-hydroxy-5-phenethyl-1,5-dihydro-pyrrol-2-one; compound with trifluoro-acetic acid;
4-Hydroxy-3-[(H-indol-3-yl)-acetyl]-5-phenethyl-1,5-dihydro-pyrrol-2-one;
3-{[1-(4-Fluoro-benzyl)-1H-indol-3-yl]-acetyl}-4-hydroxy-5-phenethyl-1,5-dihydro-pyrrol-2-one;
4-Hydroxy-3-(indol-1-yl-acetyl)-5-phenethyl-1,5-dihydro-pyrrol-2-one;
4-Hydroxy-3-(3-1H-indol-3-yl-propionyl)-5-phenethyl-1,5-dihydro-pyrrol-2-one;
3-(2-Benzo[b]thiophen-3-yl-acetyl)-4-hydroxy-5-phenethyl-1,5-dihydro-pyrrol-2-one;
3-(3,3-Diphenyl-propionyl)-4-hydroxy-5-phenethyl-1,5-dihydro-pyrrol-2-one;
3-(2,3-Diphenyl-propionyl)-4-hydroxy-5-phenethyl-1,5-dihydro-pyrrol-2-one;
3-(Carbazol-9-yl-acetyl)-4-hydroxy-5-phenethyl-1,5-dihydro-pyrrol-2-one;
4-Hydroxy-3 (R,S)-[2-(6-methoxy-naphthalen-2-yl)-propionyl]-5(R,S)-phenethyl-1,5-dihydro-pyrrol-2-one;
[1-(4-Benzyloxy-benzyl)-2-(4-hydroxy-2-oxo-5(R,S)-phenethyl-2,5-dihydro-1H-pyrrol-3-yl)-2(R,S)-oxo-ethyl]-carbamic acid tert-butyl ester;
Rac-4-hydroxy-3-(indol-1-yl-acetyl)-5-phenethyl-1,5-dihydro-pyrrol-2-one; and
Rac-3-(carbazol-9-yl-acetyl)-4-hydroxy-5-phenethyl-1,5-dihydro-pyrrol-2-one.
